SEQUENCE LISTING (33 sequences)

SEQ ID NO: 1 - DNA, 20 bp, Artificial Sequence
  spacer element sequence polynucleotide DNA-AAVS1
ggggccactagggacaggat

SEQ ID NO: 2 - RNA, 20 nt, Artificial Sequence
  spacer element sequence polynucleotide RNA-AAVS1
ggggccacuagggacaggau

SEQ ID NO: 3 - DNA, 23 bp, Artificial Sequence
  Oligonucleotide Primer
agtaataatacgactcactatag

SEQ ID NO: 4 - DNA, 58 bp, Artificial Sequence
  Oligonucleotide Primer
taatacgactcactatagggggccactagggacaggatgttttagagctagaaatagc

SEQ ID NO: 5 - DNA, 50 bp, Artificial Sequence
  Oligonucleotide Primer
gttttagagctagaaatagcaagttaaaataaggctagtccgttatcaac

SEQ ID NO: 6 - DNA, 50 bp, Artificial Sequence
  Oligonucleotide Primer
aaaaaaagcaccgactcggtgccactttttcaagttgataacggactagc

SEQ ID NO: 7 - DNA, 23 bp, Artificial Sequence
  Oligonucleotide Primer
aaaaaaagcaccgactcggtgcc

SEQ ID NO: 8 - DNA, 43 bp, Artificial Sequence
  Oligonucleotide Primer
agtaataatacgactcactatagggggccactagggacaggat

SEQ ID NO: 9 - DNA, 42 bp, Artificial Sequence
  Oligonucleotide Primer
ggggccactagggacaggatgtctcagagctatgctgtcctg

SEQ ID NO: 10 - DNA, 22 bp, Artificial Sequence
  Oligonucleotide Primer
caggacagcatagctctgagac

SEQ ID NO: 11 - DNA, 58 bp, Artificial Sequence
  Oligonucleotide Primer
agtaataatacgactcactatagcaggacagcatagcaagttgagataaggctagtcc

SEQ ID NO: 12 - DNA, 58 bp, Artificial Sequence
  Oligonucleotide Primer
agtaataatacgactcactatagtctcagagctatgctgtcctggaaacaggacagca

SEQ ID NO: 13 - DNA, 42 bp, Artificial Sequence
  Oligonucleotide Primer
gcaggacagcatagcaagttgagataaggctagtccgttatc

SEQ ID NO: 14 - DNA, 57 bp, Artificial Sequence
  Oligonucleotide Primer
tatgctgtcctggaaacaggacagcatagcaagttgagataaggctagtccgttatc

SEQ ID NO: 15 - DNA, 50 bp, Artificial Sequence
  Oligonucleotide Primer
aagcaccgactcggtgccactttttcaagttgataacggactagccttat

SEQ ID NO: 16 - DNA, 20 bp, Artificial Sequence
  Oligonucleotide Primer
aagcaccgactcggtgccac

SEQ ID NO: 17 - DNA, 20 bp, Artificial Sequence
  Oligonucleotide Primer
ccccgttctcctgtggattc

SEQ ID NO: 18 - DNA, 20 bp, Artificial Sequence
  Oligonucleotide Primer
atcctctctggctccatcgt

SEQ ID NO: 19 - RNA, 98 nt, Artificial Sequence
  Cas-associated RNA-1
gucucagagcuaugcuguccuggaaacaggacagcauagcaaguugagauaaggcuaguccguuaucaacuugaaaaaguggcaccgagucggugcuu

SEQ ID NO: 20 - protein, 1368 aa, Streptococcus pyogenes serotype M1 [sequence omitted]

SEQ ID NO: 21 - RNA, 77 nt, Artificial Sequence
  Cas-associated RNA-2
guuuuagagcuagaaauagcaaguuaaaauaaggcuaguccguuaucaacuugaaaaaguggcaccgagucggugcu

SEQ ID NO: 22 - RNA, 20 nt, Artificial Sequence
  spacer element sequence polynucleotide RNA-AAVS1
ggggccacuagggacaggau

SEQ ID NO: 23 - RNA, 25 nt, Pseudomonas aeruginosa
  misc_feature (1)..(25): Csy4 Hairpin Sequence
ggagaguucacugccguauaggcag

SEQ ID NO: 24 - RNA, 40 nt, Artificial Sequence
  linker sequence
cuaagaaugcucuuccgaucugcuacucuaagcauaucgu

SEQ ID NO: 25 - RNA, 30 nt, Artificial Sequence
  linker sequence
ugcucuuccgaucugcuacucuaagcauau

SEQ ID NO: 26 - RNA, 20 nt, Artificial Sequence
  linker sequence
ugcucuuccgaucugcuacu

SEQ ID NO: 27 - RNA, 10 nt, Artificial Sequence
  linker sequence
aucugcuacu

SEQ ID NO: 28 - RNA, 20 nt, Artificial Sequence
  spacer element sequence polynucleotide RNA-AAVS1
ucuguccccuccaccccaca

SEQ ID NO: 29 - RNA, 21 nt, Artificial Sequence
  spacer element sequence polynucleotide RNA-AAVS1
ucuguccccuccaccccacag

SEQ ID NO: 30 - RNA, 19 nt, Artificial Sequence
  Cas-associated RNA-3
aauuucuacucuuguagau

SEQ ID NO: 31 - DNA, 52 bp, Artificial Sequence
  AAVS-1 primer with adapter sequence
cactctttccctacacgacgctcttccgatcttctggcaaggagagagatgg

SEQ ID NO: 32 - DNA, 48 bp, Artificial Sequence
  AAVS-1 primer with adapter sequence
ggagttcagacgtgtgctcttccgatcttatattcccagggccggtta

SEQ ID NO: 33 - protein, 1307 aa, Acidaminococcus sp.
  MISC_FEATURE (1)..(1307): Cpf1 protein
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

(19) United States
(12) Patent Application Publication
(10) Pub. No.: US 2016/0362667 A1
(43) Pub. Date: Dec. 15, 2016

(54) CRISPR-Cas Compositions and Methods

(71) Applicant: Caribou Biosciences, Inc., Berkeley, CA (US)

(72) Inventors: Paul Daniel Donohoue, Berkeley, CA (US); Andrew Paul May, San Francisco, CA (US); Katharina Friederike Sonja Stengel, Oakland, CA (US)

(21) Appl. No.: 15/178,560

(22) Filed: Jun. 9, 2016

Related U.S. Application Data
(60) Provisional application No. 62/173,907, filed on Jun. 10, 2015.
(60) Provisional application No. 62/173,912, filed on Jun. 10, 2015.

(51) Int. Cl. C12N 9/22; C12N 15/90; C12P 19/34

(52) U.S. Cl. CPC C12N 9/22; C12P 19/34; C12N 15/907; C12Y 301/00; C12N 2800/80

(57) ABSTRACT

Class 2 CRISPR-Cas nucleoprotein complexes are disclosed comprising a Class 2 CRISPR-Cas protein, a CRISPR-Cas associated polynucleotide lacking a spacer element (casPN), and a distinct spacer element sequence polynucleotide (sesPN) comprising a target nucleic acid binding sequence. These complexes are capable of site-directed binding to a target nucleic acid complementary to the target nucleic acid binding sequence of the sesPN. The Class 2 CRISPR-Cas nucleoprotein complexes facilitate site-specific modifications, including cleavage and mutagenesis, of a target nucleic acid sequence. Polynucleotide sequences, expression cassettes, vectors, compositions, and kits for carrying out a variety of methods are also described. Furthermore, the present specification provides guidance for methods of regulating expression of a target nucleic acid sequence, production of genetically modified cells, compositions of modified cells, transgenic organisms, pharmaceutical compositions, as well as a variety of other compositions and methods involving the Class 2 CRISPR-Cas nucleoprotein complexes comprising casPNs, sesPNs, and Cas proteins.

### CROSS-REFERENCE TO RELATED APPLICATIONS

This application claims the benefit of U.S. Provisional Patent Application Ser. No. 62/173,907, filed 10 Jun. 2015, now pending, and U.S. Provisional Application Ser. No. 62/173,912, filed 10 Jun. 2015, now pending, which applications are herein incorporated by reference in their entireties.

### STATEMENT REGARDING FEDERALLY SPONSORED RESEARCH OR DEVELOPMENT

Not applicable.

### SEQUENCE LISTING

The present application contains a Sequence Listing that has been submitted electronically in ASCII format and is hereby incorporated by reference in its entirety. The ASCII copy, created on 9 Jun. 2016, is named CBI015-10_ST25.txt and is 30 kb in size.

### TECHNICAL FIELD

The present invention relates to engineered Class 2 CRISPR-Cas systems.

### BACKGROUND

Clustered Regularly Interspaced Short Palindromic Repeats (CRISPR) are prokaryotic immune systems first discovered by Ishino in E. coli. (Ishino, et al., Journal of Bacteriology 169(12): 5429-5433 (1987)). These system provide immunity in bacteria and archaea against viruses and plasmids by targeting the nucleic acids of the viruses and plasmids in a sequence-specific manner.

There are two main stages involved in these immune systems; the first is acquisition and the second is interference. The first stage involves cutting the genome of invading viruses and plasmids and integrating segments of this into the CRISPR locus of the bacteria and archaea. The segments to be integrated into the genome are known as protospacers and help in protecting the organism from subsequent attack by the same virus or plasmid. The second stage involves attacking an invading virus or plasmid. This stage relies upon the integrated sequences, called spacers, being transcribed to RNA and following some processing this RNA then hybridizes with a complementary sequence in the DNA or RNA of an invading polynucleotide (e.g., a virus or a plasmid) while also associating with a protein, or protein complex, that effectively binds and/or cleaves the DNA or RNA.

There are several different CRISPR-Cas systems and the nomenclature and classification of these has changed as the systems are further characterized. In Class 2 Type II systems there are two strands of RNA that are part of the CRISPR-Cas system: a CRISPR RNA (crRNA) and a transactivating CRISPR RNA (tracrRNA). The tracrRNA hybridizes to a complementary region of pre-crRNA facilitating maturation of the pre-crRNA to crRNA by an RNase III enzyme. The duplex formed by the tracrRNA and crRNA is recognized by, and associates with a protein, Cas9, which is directed to a target nucleic acid by a sequence of the crRNA that is complementary to, and hybridizes with, a sequence in the target nucleic acid. It has been demonstrated that these minimal components of the RNA-based immune system can be reprogrammed to target DNA in a site-specific manner by using a single protein and two RNA guide sequences or a single RNA molecule.

In Class 2 Type V CRISPR systems it has also been demonstrated that the Cas protein Cpf1 can be reprogrammed to target DNA in a site-specific manner with a single crRNA sequence.

The CRISPR-Cas system is superior to other methods of genome editing such as endonucleases, meganucleases, zinc finger nucleases, and transcription activator-like effector nucleases (TALENs), which may require de novo protein engineering for every new target locus.

### SUMMARY OF THE INVENTION

As used herein and described in detail below, the term “sesPN” refers to a “spacer element sequence polynucleotide” of the present invention and the term “casPN” refers to a “Cas-associated polynucleotide (lacking a spacer element).”

The present invention relates to compositions and methods relating to Class 2 CRISPR-Cas associated polynucleotides lacking a spacer element (casPNs) and distinct spacer element sequence polynucleotides (sesPNs) comprising a target nucleic acid binding sequence.

In one aspect, the present invention relates to a Class 2 CRISPR-Cas nucleoprotein complex. The complex comprises a Class 2 CRISPR-Cas protein, a Class 2 CRISPR-Cas associated polynucleotide lacking a spacer element (casPN), and a distinct spacer element sequence polynucleotide (sesPN) comprising a target nucleic acid binding sequence. The Class 2 CRISPR-Cas nucleoprotein complex is capable of site-directed binding to a target nucleic acid complementary to the target nucleic acid binding sequence of the sesPN.

In some embodiments the casPN comprises RNA, DNA, analogs thereof, or combinations thereof. In a preferred embodiment, the casPN comprises RNA, DNA, or combinations thereof.

In some embodiments the sesPN comprises RNA, DNA, analogs thereof, or combinations thereof. In a preferred embodiment, the sesPN comprises RNA, DNA, or combinations thereof.

A sesPN and a casPN of a Class 2 CRISPR-Cas nucleoprotein complex can both comprise the same type of polynucleotide (e.g., RNA, DNA, or combinations thereof) or a sesPN and a casPN may each comprise different types of polynucleotides.

In one embodiment the Cas protein of a Class 2 CRISPR-Cas nucleoprotein complex comprises a Cas protein selected from the group consisting of a Cas9 protein, a Cas9-like protein, a protein encoded by a Cas9 ortholog, a Cas9-like synthetic protein, a Cpf1 protein, a protein encoded by a Cpf1 ortholog, a Cpf1-like synthetic protein, a C2c1 protein, a C2c2 protein, a C2c3 protein, and variants and modifications thereof. In a first preferred embodiment, the Cas protein is a Class 2 Type II CRISPR Cas9. In a second preferred embodiment, the Cas protein is a Class 2 Type V CRISPR Cpf1. In some embodiments, a Cas protein comprises an enzymatically inactive Cas protein variant, for example, a dCas9 or a dCpf1. In other embodiments, a Cas protein comprises a Cas protein having modified enzymatic activity, for example, reduced enzymatic activity.

Additional embodiments of the present invention include a Class 2 CRISPR-Cas nucleoprotein complex wherein (i) the sesPN and/or the casPN further comprises a nucleic acid binding protein binding sequence, and (ii) the Cas protein comprises a fusion protein comprising the Cas protein and a nucleic acid binding protein or protein domain that binds the nucleic acid binding protein binding sequence of the sesPN or the casPN. Typically, if both the sesPN and the casPN comprise a nucleic acid binding protein binding sequence, the nucleic acid binding protein binding sequences do not bind the same nucleic acid binding protein/protein domain and the fusion protein comprises both of the nucleic acid binding proteins/domains. In one example, a nucleic acid binding protein or protein domain comprises a dCsy4 protein and the nucleic acid binding protein binding sequence comprises a Csy4 RNA binding sequence, that is, a RNA binding sequence to which the dCsy4 protein is capable of binding. A number of different Csy4 proteins each having a different corresponding Csy4 RNA binding sequence are known in the art.

In other embodiments the present invention relates to a Class 2 CRISPR-Cas nucleoprotein complex wherein (i) the Cas protein comprises an engineered Cas protein comprising a Cys substitution of a non-Cys amino acid residue or an inserted Cys amino acid, (ii) the sesPN comprises a thiol cross-linking moiety, and (iii) the engineered Cas protein substituted Cys amino acid residue or inserted Cys amino acid is covalently bound to the sesPN thiol cross-linking moiety. A similar embodiment relates to a Class 2 CRISPR-Cas nucleoprotein complex wherein (i) the Cas protein comprises an engineered Cas protein comprising a Cys substitution of a non-Cys amino acid residue or an inserted Cys amino acid, (ii) the casPN comprises a thiol cross-linking moiety, and (iii) the engineered Cas protein substituted Cys amino acid residue or inserted Cys amino acid is covalently bound to the casPN thiol cross-linking moiety. Examples of thiol cross-linking moiety include, but are not limited to, 5′ thiol C6, dithiol phosphoramidite, and 3′ thiol C3.

When a sesPN and a casPN are both modified with a cross-linking moiety, orthogonality is maintained relative to the two binding sites of the cross-linking moiety in a Cas protein to which the sesPN and the casPN are cross-linked. For example, the sesPN is modified with a thiol cross-linking moiety that links the sesPN to a Cys in the Cas protein and the casPN is modified with a photoactive cross-linking moiety that links the casPN to a photoreactive amino acid in the Cas protein. In another embodiment, a sesPN, for example, is modified with a cross-linking moiety that binds to an amino acid residue in a Cas protein, wherein the Cas protein comprises a fusion protein comprising a Cas protein and a nucleic acid binding protein or protein domain. A casPN comprises a nucleic acid binding protein binding sequence to which the nucleic acid binding protein or protein domain binds. In a related embodiment, a casPN comprises the cross-linking moiety and the sesPN comprises a nucleic acid binding protein binding sequence.

A large number of affinity tags useful in tethering a sesPN and/or a casPN to a Cas protein or a fusion protein comprising a Cas protein are disclosed in the present specification.

In another aspect, the present invention relates to a method of binding a target nucleic acid comprising contacting a nucleic acid comprising the target nucleic acid with a Class 2 CRISPR-Cas nucleoprotein complex comprising a sesPN, a casPN, and a Cas protein (e.g., a Class 2 CRISPR-Cas nucleoprotein complex of the present invention as described above) thereby facilitating binding of the Class 2 CRISPR-Cas nucleoprotein complex to the target nucleic acid. In one embodiment, genomic DNA of a cell comprises the target nucleic acid. In additional embodiments, the Cas protein comprises a Cas protein that is enzymatically inactive or a Cas protein having modified enzymatic activity, for example, reduced enzymatic activity.

In a further aspect, the present invention relates to a method of cutting a target nucleic acid comprising contacting a nucleic acid comprising the target nucleic acid with a Class 2 CRISPR-Cas nucleoprotein complex comprising a sesPN, a casPN, and a Cas protein (e.g., a Class 2 CRISPR-Cas nucleoprotein complex of the present invention as described above), thereby facilitating binding of the Class 2 CRISPR-Cas nucleoprotein complex to the target nucleic acid, wherein the bound Class 2 CRISPR-Cas nucleoprotein complex cuts the target nucleic acid.

An additional aspect of the present invention relates to a kit comprising a Class 2 CRISPR-Cas nucleoprotein complex comprising a sesPN, a casPN, and a Cas protein (e.g., a Class 2 CRISPR-Cas nucleoprotein complex of the present invention as described above), and a buffer.

Another aspect of the present invention relates to a composition comprising a Class 2 CRISPR-Cas associated polynucleotide lacking a spacer element (casPN), wherein the casPN is capable of associating with (i) a Class 2 CRISPR-Cas protein and (ii) a distinct spacer element sequence polynucleotide (sesPN) comprising a target nucleic acid binding sequence, thereby forming a Class 2 CRISPR-Cas nucleoprotein complex. The Class 2 CRISPR-Cas nucleoprotein complex is capable of site-directed binding to a target nucleic acid complementary to the target nucleic acid binding sequence of the sesPN. In some embodiments the casPN comprises an affinity tag as described herein. In some embodiments a kit comprises the composition comprising the Class 2 CRISPR-Cas associated polynucleotide lacking a spacer element (casPN) and a buffer. In additional embodiments the kit further comprises a cognate Class 2 CRISPR-Cas protein or a polynucleotide encoding the Class 2 CRISPR-Cas protein. Further embodiments of the kit comprise a distinct spacer element sequence polynucleotide (sesPN) comprising a target nucleic acid binding sequence.

An additional aspect of the present invention relates to a composition comprising a Class 2 CRISPR-Cas associated polynucleotide lacking a spacer element (casPN), wherein the casPN is capable of associating with a Class 2 CRISPR-Cas protein to form a casPN/Cas nucleoprotein complex, and the associating forms a nucleic acid sequence binding channel in the casPN/Cas protein complex capable of binding a nucleic acid sequence. In some embodiments the casPN comprises an affinity tag as described herein. In some embodiments a kit comprises the composition comprising the Class 2 CRISPR-Cas associated polynucleotide lacking a spacer element (casPN) and a buffer. In additional embodiments the kit further comprises a cognate Class 2 CRISPR-Cas protein or a polynucleotide encoding the Class 2 CRISPR-Cas protein. Further embodiments of the kit comprise a distinct spacer element sequence polynucleotide (sesPN) comprising a target nucleic acid binding sequence.

Such methods of binding a target nucleic acid or cutting a target nucleic acid are carried out in vitro, in cell (e.g., in host cells), ex vivo (e.g., in cells removed from a subject), and in vivo (e.g., in a subject, in one embodiment a non-human subject).

Additional aspects and other embodiments of the present invention using a sesPN, a casPN, and/or a Cas protein as described herein will readily occur to those of ordinary skill in the art in view of the teachings of the present specification.

### BRIEF DESCRIPTION OF THE FIGURES

FIG. 1A, FIG. 1B, FIG. 1C and FIG. 1D present illustrative examples of wild-type Class 2 CRISPR-Cas associated RNAs. FIG. 1A and FIG. 1C illustrate two-RNA component Class 2 Type II CRISPR-Cas9 systems comprising a crRNA (FIG. 1A, 101; FIG. 1C, 101) and a tracrRNA (FIG. 1A, 102; FIG. 1C, 102). FIG. 1B illustrates the formation of base-pair hydrogen bonds between the crRNA and the tracrRNA of FIG. 1A to form secondary structure (see U.S. Published Patent Application No. 2014-0068797, published 6 Mar. 2014; see also Jinek M., et al., “A programmable dual-RNA-guided DNA endonuclease in adaptive bacterial immunity,” Science, 2012; 337:816-21). FIG. 1B presents an overview of and nomenclature for secondary structural elements of the crRNA and tracrRNA of an exemplary Streptococcus pyogenes Class 2 Type II CRISPR-Cas9 system including the following: a spacer element (FIG. 1B, 101); a first stem element comprising a lower stem element (FIG. 1B, 103), a bulge element comprising unpaired nucleotides (FIG. 1B, 104), and an upper stem element (FIG. 1B, 105); a nexus element (FIG. 1B, 106); a first hairpin element (FIG. 1B, 107); and a second hairpin element (FIG. 1B, 108). FIG. 1D illustrates the formation of base-pair hydrogen bonds between the crRNA and the tracrRNA of FIG. 1C to form secondary structure. FIG. 1D presents an overview of and nomenclature for secondary structural elements of the crRNA and tracrRNA of an exemplary Campylobacter lari Class 2 Type II CRISPR-Cas9 system including the following: a spacer element (FIG. 1D, 101); a first stem element (FIG. 1D, 109), a nexus element (FIG. 1D, 106); a first hairpin element (FIG. 1D, 107); and a second hairpin element (FIG. 1D, 108). The figures are not proportionally rendered nor are they to scale. The locations of indicators are approximate.

FIG. 1E and FIG. 1F illustrate examples of Class 2 Type II CRISPR-Cas polynucleotides of the present invention as described herein comprising a sesPN (spacer element sequence polynucleotide) (FIG. 1E, 101; FIG. 1F, 101) and a casPN (Cas-associated polynucleotide (lacking a spacer element)) comprising two polynucleotides (FIG. 1E, 110; FIG. 1F, 110). The figures are not proportionally rendered nor are they to scale.

FIG. 2A and FIG. 2B show additional examples of Class 2 Type II CRISPR-Cas9 associated RNA. The figures illustrate a single guide RNA (sgRNA) wherein the crRNA is covalently joined to the tracrRNA and forms RNA polynucleotide secondary structure through base-pair hydrogen bonding (see, e.g., U.S. Published Patent Application No. 2014-0068797, published 6 Mar. 2014). FIG. 2A presents an overview of and nomenclature for secondary structural elements of a sgRNA of an exemplary Streptococcus pyogenes Class 2 Type II CRISPR-Cas9 system including the following: a spacer element (FIG. 2A, 201); a first stem element comprising a lower stem element (FIG. 2A, 202), a bulge element comprising unpaired nucleotides (FIG. 2A, 205), and an upper stem element (FIG. 2A, 203); a loop element (FIG. 2A, 204) comprising unpaired nucleotides; a nexus element (FIG. 2A, 206); a first hairpin element (FIG. 2A, 207); and a second hairpin element (FIG. 2A, 208). (See, e.g., FIGS. 1 and 3 of Briner, A. E., et al., “Guide RNA Functional Modules Direct Cas9 Activity and Orthogonality,” Molecular Cell Volume 56, Issue 2, 23 Oct. 2014, Pages 333-339.) FIG. 2B presents an overview of and nomenclature for secondary structural elements of a sgRNA of an exemplary Campylobacter lari Class 2 Type II CRISPR-Cas9 system including the following: a spacer element (FIG. 2B, 201); a first stem element (FIG. 2B, 209); a loop element (FIG. 2B, 204) comprising unpaired nucleotides; a nexus element (FIG. 2B, 206); a first hairpin element (FIG. 2B, 207); and a second hairpin element (FIG. 2B, 208). The figures are not proportionally rendered nor are they to scale. The locations of indicators are approximate.

FIG. 2C and FIG. 2D illustrate examples of Class 2 Type II CRISPR-Cas polynucleotides of the present invention comprising a sesPN (FIG. 2C, 201; FIG. 2D, 201) and a casPN (FIG. 2C, 210, FIG. 2D, 210) as described herein. The figures are not proportionally rendered nor are they to scale. FIG. 2C, 210, is one embodiment of the casPNs of the present invention and various modifications of the casPNs are described herein. The elements of an exemplary casRNA in a linear sequence comprise one single-strand RNA polynucleotide having a 5′ end and a 3′ end, comprising in the 5′ to 3′ direction the following contiguous sequences: a lower stem sequence 1, a bulge sequence 1, an upper stem sequence 1, a loop sequence, an upper stem sequence 2, a bulge sequence 2, a lower stem sequence 2, a nexus sequence 1, a nexus sequence 2, a single-strand sequence, a first hairpin sequence 1, a first hairpin sequence 2, a second hairpin sequence 1, and a second hairpin sequence 2; wherein (i) the upper stem sequence 1 and the upper stem sequence 2 form an upper stem element by base-pair hydrogen bonding between the upper stem sequence 1 and the upper stem sequence 2 (compare FIG. 2A, 203), (ii) the lower stem sequence 1 and lower stem sequence 2 form the lower stem element by base-pair hydrogen bonding between the lower stem sequence 1 and lower stem sequence 2 (compare FIG. 2A, 202), (iii) a nexus sequence comprising a nexus-stem sequence 1 and nexus stem sequence 2 that form a nexus stem structure by base-pair hydrogen bonding between the nexus-stem sequence 1 and the nexus-stem sequence 2 (compare FIG. 2A, 206), (iv) the first hairpin sequence 1 and the first hairpin sequence 2 form the first hairpin by base-pair hydrogen bonding between the first hairpin sequence 1 and the first hairpin sequence 2 (compare FIG. 2A, 207), and (v) the second hairpin sequence a and the second hairpin sequence 2 form the second hairpin by base-pair hydrogen bonding between the second hairpin 1 sequence and the second hairpin sequence 2 (compare FIG. 2A, 208).

FIG. 3A and FIG. 3B illustrate two examples of Class 2 Type V CRISPR-Cas crRNAs. FIG. 3A presents an overview of and nomenclature for secondary structural elements of the crRNA of an exemplary Acidaminococcus spp. Class 2 Type V CRISPR-Cas (Cpf1) system including the following: a stem element sequence 1 (FIG. 3A, 303), a loop sequence (FIG. 3A, 304), a stem element sequence 2 (FIG. 3A, 305), and a spacer element (FIG. 3A, 302), wherein the stem element sequence 1 and the stem element sequence 2 form a stem element (FIG. 3A, 301) by base-pair hydrogen bonding between the stem element sequence 1 and the stem element sequence 2. The hairpin structure comprising the stem element sequence 1 (FIG. 3A, 303), the loop sequence (FIG. 3A, 304), and the stem element sequence 2 (FIG. 3A, 305) is referred to herein as a “pseudoknot element.” FIG. 3B presents secondary structural elements for an alternative Class 2 Type V CRISPR-Cas crRNA including the following: a stem element sequence 1 (FIG. 3B, 303), a stem element sequence 2 (FIG. 3B, 305), and a spacer element (FIG. 3B, 302), wherein the stem element sequence 1 and the stem element sequence 2 form a stem element (FIG. 3B, 301) by base-pair hydrogen bonding between the stem element sequence 1 and the stem element sequence 2. The figures are not proportionally rendered nor are they to scale. The locations of indicators are approximate.

FIG. 3C and FIG. 3D illustrate examples of Class 2 Type V CRISPR-Cas polynucleotides of the present invention as described herein comprising a sesPN (FIG. 3C, 302) and a casPN (FIG. 3C, 306) and in an alternative embodiment a sesPN (FIG. 3D, 302) and a casPN comprising two polynucleotide sequences (FIG. 3D, 306). The figures are not proportionally rendered nor are they to scale.

FIG. 4A illustrates a Class 2 Type II CRISPR-Cas sgRNA (FIG. 4A, 401) (compare FIG. 2A).

FIG. 4B illustrates an example of a Class 2 Type II CRISPR-Cas ribonucleoprotein complex bound to a double-stranded DNA comprising a target DNA sequence, wherein the ribonucleoprotein complex has cut both strands of the double-stranded target DNA sequence. In FIG. 4B, the sgRNA (FIG. 4B, 401) is complexed with a cognate Cas9 protein (FIG. 4B, 402). The box with dashed lines (FIG. 4B, 403) illustrates the spacer element of the sgRNA hybridized to the complementary target DNA sequence in the 3′ to 5′ DNA strand (FIG. 4B, 404). The location of the cut made by the Cas9 protein of the ribonucleoprotein complex is indicated by the arrow (FIG. 4B, 407). The protospacer adjacent motif (PAM) (FIG. 4B, 406) in the double-stranded DNA is present in 5′ to 3′ DNA strand (FIG. 4B, 405). The figures are not proportionally rendered nor are they to scale. The locations of indicators are approximate.

FIG. 5A illustrates a sesPN (FIG. 5A, 502) and a casPN (FIG. 5A, 501) of the present invention.

FIG. 5B illustrates an example of a Class 2 Type II CRISPR-Cas ribonucleoprotein complex of the present invention bound to a double-stranded DNA comprising a target DNA sequence, wherein the ribonucleoprotein complex has cut both strands of the double-stranded target DNA sequence. In FIG. 5B, a casRNA (FIG. 5B, 501) and a sesRNA (FIG. 5B, 502) are complexed with a cognate Cas9 protein (FIG. 5B, 503). The box with dashed lines (FIG. 5B, 504) illustrates the sesRNA hybridized to the complementary target DNA sequence in the 3′ to 5′ DNA strand (FIG. 5B, 505). The location of the cut made by the Cas9 protein of the ribonucleoprotein complex is indicated by the arrow (FIG. 5B, 508). The PAM (FIG. 5B, 507) in the double-stranded DNA is present in 5′ to 3′ DNA strand (FIG. 5B, 506). The figures are not proportionally rendered nor are they to scale. The locations of indicators are approximate.

FIG. 6A illustrates a Class 2 Type V CRISPR-Cas crRNA (FIG. 6A, 601) (compare FIG. 3A).

FIG. 6B illustrates an example of a Class 2 Type V CRISPR-Cas ribonucleoprotein complex bound to a double-stranded DNA comprising a target DNA sequence, wherein the ribonucleoprotein complex has cut both strands of the double-stranded target DNA sequence. In FIG. 6B, the crRNA (FIG. 6B, 601) is complexed with a cognate Cpf1 protein (FIG. 6B, 602). The box with dashed lines (FIG. 6B, 603) illustrates the spacer element of the crRNA hybridized to the complementary target DNA sequence in the 3′ to 5′ DNA strand (FIG. 6B, 604). The locations of the cuts made by the Cpf1 protein of the ribonucleoprotein complex are indicated by the arrows (FIG. 6B, 606). The PAM (FIG. 6B, 607) in the double-stranded DNA is present in 5′ to 3′ DNA strand (FIG. 6B, 605). The figures are not proportionally rendered nor are they to scale. The locations of indicators are approximate.

FIG. 7A illustrates a sesPN (FIG. 7A, 702) and a casPN (FIG. 7A, 701) of the present invention.

FIG. 7B illustrates an example of a Class 2 Type V CRISPR-Cas ribonucleoprotein complex of the present invention bound to a double-stranded DNA comprising a target DNA sequence, wherein the ribonucleoprotein complex has cut both strands of the double-stranded target DNA sequence. In FIG. 7B, a casRNA (FIG. 7B, 701) and a sesRNA (FIG. 7B, 702) are complexed with a cognate Cpf1 protein (FIG. 7B, 703). The box with dashed lines (FIG. 7B, 704) illustrates the sesRNA hybridized to the complementary target DNA sequence in the 3′ to 5′ DNA strand (FIG. 7B, 705). The locations of the cuts made by the Cpf1 protein of the ribonucleoprotein complex are indicated by the arrow (FIG. 7B, 707). The PAM (FIG. 7B, 708) in the double-stranded DNA is present in 5′ to 3′ DNA strand (FIG. 7B, 706). The Cpf1 protein comprises an engineered Cpf1 protein having a cysteine (Cys) substitution (FIG. 7B, 709) of a non-Cys amino acid residue and the sesRNA comprises a thiol cross-linking moiety (FIG. 7B, 710). The substituted Cys amino acid residue of the engineered Cpf1 protein is covalently bound through the S—S bond (FIG. 7B, 711) to the sesRNA thiol cross-linking moiety. The S—S bond between the substituted Cys residue and the sesRNA thiol cross-linking moiety shows an example of a method that is used to bring the sesRNA into proximity with the RNA binding channel of the Cpf1 protein. The figures are not proportionally rendered nor are they to scale. The locations of indicators are approximate.

FIG. 8 is an oligonucleotide table that sets forth the sequences of oligonucleotides used in the Examples of the present specification.

FIG. 9A, FIG. 9B, and FIG. 9C present exemplary thiol functionalities as follows: FIG. 9A, 5′ Thiol C6; FIG. 9B, dithiol phosphoramidite, DTPA; and FIG. 9C 3′ Thiol C3. Arrows indicate the sites of reduction of disulfide bonds.

FIG. 10 illustrates an example of a Class 2 Type II CRISPR-Cas ribonucleoprotein complex of the present invention bound to a double-stranded DNA comprising a target DNA sequence, wherein the ribonucleoprotein complex has cut both strands of the double-stranded target DNA sequence. In FIG. 10, a casRNA (FIG. 10, 1001) and a sesRNA (FIG. 10, 1005) are complexed with a cognate Cas9 protein (FIG. 10, 1000). The sesRNA is hybridized to the complementary target DNA sequence in the 3′ to 5′ DNA strand (FIG. 10, 1006). The location of the cut made by the Cas9 protein of the ribonucleoprotein complex is indicated by the arrow (FIG. 10, 1009). The PAM (FIG. 10, 1008) in the double-stranded DNA is present in 5′ to 3′ DNA strand (FIG. 10, 1007). The Cas protein comprises an engineered Cas protein having a cysteine (Cys) substitution (FIG. 10, 1002) of a non-Cys amino acid residue and the sesRNA comprises a thiol cross-linking moiety (FIG. 10, 1004). The substituted Cys amino acid residue of the engineered Cas9 protein is covalently bound through the S—S bond (FIG. 10, 1003) to the sesRNA thiol cross-linking moiety. The S—S bond between the substituted Cys residue and the sesRNA thiol cross-linking moiety shows an example of a method that is used to bring the sesRNA into proximity with the RNA binding channel of the Cas9 protein. The figures are not proportionally rendered nor are they to scale. The locations of indicators are approximate.

FIG. 11 illustrates an example of a Class 2 Type II CRISPR-Cas ribonucleoprotein complex of the present invention bound to a double-stranded DNA comprising a target DNA sequence, wherein the ribonucleoprotein complex has cut both strands of the double-stranded target DNA sequence. In FIG. 11, a casRNA (FIG. 11, 1101) and a sesRNA (FIG. 11, 1103) comprising a Csy4 RNA binding sequence (the hairpin near the 5′ end of the sesRNA) are complexed with a cognate Cas9 protein (FIG. 11, 1100). The sesRNA is hybridized to the complementary target DNA sequence in the 3′ to 5′ DNA strand (FIG. 11, 1104). The location of the cut made by the Cas9 protein of the ribonucleoprotein complex is indicated by the arrow (FIG. 11, 1107). The PAM (FIG. 11, 1106) in the double-stranded DNA is present in 5′ to 3′ DNA strand (FIG. 11, 1105). The Cas protein comprises a fusion protein comprising the Cas9 protein (FIG. 11, 1100) and a dCsy4 (enzymatically inactive Csy4) domain (FIG. 11, 1102) that binds the Csy4 RNA binding sequence of the sesRNA. The binding of the dCsy4 domain of the fusion protein to the Csy4 RNA binding sequence shows another example of a method that is used to bring the sesRNA into proximity with the RNA binding channel of the Cas9 protein. The figures are not proportionally rendered nor are they to scale. The locations of indicators are approximate.

FIG. 12A and FIG. 12B relate to structural information for a sgRNA/Cas protein complex and a Cas protein, respectively. FIG. 12A provides a model based on the crystal structure of Streptococcus pyogenes Cas9 (SpyCas9) in an active complex with sgRNA (single guide RNA) (Anders C., et al., “Structural basis of PAM-dependent target DNA recognition by the Cas9 endonuclease,” Nature, 2014; 513(7519):569-73). Structural studies of the SpyCas9 showed that the protein exhibits a bi-lobed architecture comprising the catalytic nuclease lobe and the alpha-helical lobe of the enzyme (See Jinek M., et al., “Structures of Cas9 endonucleases reveal RNA-mediated conformational activation,” Science, 2014; 343(6176):1247997; Anders C., et al., “Structural basis of PAM-dependent target DNA recognition by the Cas9 endonuclease,” Nature, 2014; 513(7519):569-73). In FIG. 12A, the alpha-helical lobe (FIG. 12A, 1200; helical domain) is shown as the darker lobe, the catalytic nuclease lobe (FIG. 12A, 1201; catalytic nuclease lobe) is shown in a light grey and the sgRNA backbone is shown in black (FIG. 12A, 1202; sgRNA). The relative location of the 3′ end of the sgRNA is indicated (FIG. 12A, 1203; 3′ end sgRNA). The spacer RNA of the sgRNA is not visible because it is surrounded by the two protein lobes. The relative location of the 5′ end of the sgRNA (FIG. 12A, 1204; 5′ end sgRNA) is indicated and the spacer RNA of the sgRNA is located in the 5′ end region of the sgRNA. A cysteine (Cys) residue (FIG. 12A, 1205; WT SpyCas9 Cys) in wild-type SpyCas9 is identified in the present disclosure as an available cross-linking site. In FIG. 12A, the catalytic nuclease lobe is shown as the lighter lobe wherein the relative positions of the RuvC (FIG. 12A, 1206; RuvC; RNase H homologous domain) and HNH nuclease (FIG. 12A, 1207; HNH; HNH nuclease homologous domain) domains are indicated. The RuvC and HNH nuclease domains, when active, each cut a different DNA strand in target DNA. The C-terminal domain (CTD) (FIG. 12A, 1208; CTD) is involved in recognition of protospacer adjacent motifs (PAM) in target DNA.

FIG. 12B presents a model of the domain arrangement of SpyCas9 relative to its primary sequence structure. In FIG. 12B, three regions of the primary sequence correspond to the RuvC domain (FIG. 12B, 1209, RuvC-I (amino acids 1-78); FIG. 12B, 1210, RuvC-II (amino acids 719-765); and FIG. 12B, 1211, RuvC-III (amino acids 926-1102)). One region corresponds to the helical domain (FIG. 12B, 1212; helical domain (amino acids 79-718). One region corresponds to the HNH domain (FIG. 12B, 1213; HNH (amino acids 766-925). One region corresponds to the CTD domain (FIG. 12B, 1214; CTD (amino acids 1103-1368). In FIG. 12B, the regions of the primary sequence corresponding to the alpha-helical lobe (FIG. 12B, 1212; alpha-helical lobe) and the Nuclease domain lobe (FIG. 12B, 1215; Nuclease domain lobe) are indicated with brackets. The figures are not proportionally rendered nor are they to scale. The locations of indicators are approximate.

FIG. 13A and FIG. 13B provide a close-up, open book view of SpyCas9. FIG. 13A presents a model of the alpha-helical lobe (FIG. 13A, 1300; helical domain) of SpyCas9 in complex with an sgRNA. The sgRNA (FIG. 13A, 1301; sgRNA) backbone is shown in grey and the spacer RNA of the sgRNA backbone is shown in black; the section of the sgRNA corresponding to the spacer RNA is also indicated by a bracket (FIG. 13A, 1302; Spacer RNA). The 3′ end of the sgRNA (FIG. 13A, 1303; 3′ end sgRNA) and the 5′ end of the sgRNA (FIG. 13A, 1304; 5′ end sgRNA) are indicated. Epitopes within the helical domain, identified in the present disclosure as available cross-linking sites, are shown in black along the length of the spacer RNA region. The black dot (FIG. 13A, 1309) corresponds to the black color of the cross-linking epitopes.

FIG. 13B presents a model of the catalytic nuclease lobe (FIG. 13B, 1305; catalytic nuclease lobe) of SpyCas9 in complex with an sgRNA. The sgRNA (FIG. 13B, 1301; sgRNA) backbone is shown in grey and the spacer RNA region of the sgRNA backbone is shown in black; the section of the sgRNA corresponding to the spacer RNA is also indicated by a bracket (FIG. 13B, 1302; Spacer RNA). The 3′ end of the sgRNA (FIG. 13B, 1303; 3′ end sgRNA) and the 5′ end of the sgRNA (FIG. 13B, 1304; 5′ end sgRNA) are indicated. Epitopes within the catalytic nuclease lobe, identified by the teachings of the present disclosure as available cross-linking sites, are shown in black along the length of the spacer RNA region. The relative positions of the RuvC domain (FIG. 13B, 1306; RuvC domain), HNH nuclease domain (FIG. 13B, 1307; HNH domain), and the CTD (FIG. 13B, 1308; CTD) in the catalytic nuclease lobe are indicated. The black dot (FIG. 13B, 1309) corresponds to the black color of the cross-linking epitopes. The figures are not proportionally rendered nor are they to scale. The locations of indicators are approximate.

FIG. 14 provides a close-up view of residue Ser590 in SpyCas9 (FIG. 14, 1400; Ser590) and a model of a sesPN (FIG. 14, 1401) as described herein. In the figure, a relevant portion of the sesPN is indicated. The distance between the side chain of Ser590 and the sesPN backbone is about 7.15 Å (FIG. 14, 1402; dotted grey line), which is a suitable distance for cross-linking. In the figure, a relevant portion of the alpha-helical lobe (FIG. 14, 1403, helical domain) is indicated. The figure is proportionally rendered nor to scale. The locations of indicators are approximate.

FIG. 15A and FIG. 15B provide an illustration of the relative locations of a sesPN and a casPN of the present invention to SpyCas9. FIG. 15A provides a close-up view of the 3′ end of the sesPN (FIG. 15A, 1500) adjacent the 5′ end of the casPN (FIG. 15A, 1501). The 5′ end of the sesPN is also indicated (FIG. 15A, 1502). FIG. 15A shows the casPN and the sesPN in complex with the helical domain (FIG. 15A, 1504) of SpyCas9. In FIG. 15A, the sesPN is shown in black and the casPN (FIG. 15A, 1503) is shown in grey; sesPN and casPN are not covalently linked to each other.

FIG. 15B provides a close up view of the helical domain of SpyCas9 (FIG. 15B, 1504) in complex with sesPN (shown in black in FIG. 15B). The 3′ end of the sesPN is indicated (FIG. 15B, 1500). Epitopes within the helical domain available for polynucleotide-protein cross-linking (as discussed in the teachings of the present disclosure), at the 3′ end of sesPN (FIG. 15B, 1500), are shown in dark grey. (The grey dot (FIG. 15B, 1505) corresponds to the grey coloring of the epitopes). The figures are not proportionally rendered nor are they to scale. The locations of indicators are approximate.

### INCORPORATION BY REFERENCE

All patents, publications, and patent applications cited in this specification are herein incorporated by reference as if each individual patent, publication, or patent application was specifically and individually indicated to be incorporated by reference in its entirety for all purposes.

### DETAILED DESCRIPTION OF THE INVENTION

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to “a primer” includes one or more primer, reference to “a recombinant cell” includes one or more recombinant cell, reference to “a cross-linking agent” includes one or more cross-linking agent, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Antibodies: A Laboratory Manual, Second edition, E. A. Greenfield, 2014, Cold Spring Harbor Laboratory Press, ISBN 978-1-936113-81-1; Culture of Animal Cells: A Manual of Basic Technique and Specialized Applications, 6th Edition, R. I. Freshney, 2010, Wiley-Blackwell, ISBN 978-0-470-52812-9; Transgenic Animal Technology, Third Edition: A Laboratory Handbook, 2014, C. A. Pinkert, Elsevier, ISBN 978-0124104907; The Laboratory Mouse, Second Edition, 2012, H. Hedrich, Academic Press, ISBN 978-0123820082; Manipulating the Mouse Embryo: A Laboratory Manual, 2013, R. Behringer, et al., Cold Spring Harbor Laboratory Press, ISBN 978-1936113019; PCR 2: A Practical Approach, 1995, M. J. McPherson, et al., IRL Press, ISBN 978-0199634248; Methods in Molecular Biology (Series), J. M. Walker, ISSN 1064-3745, Humana Press; RNA: A Laboratory Manual, 2010, D. C. Rio, et al., Cold Spring Harbor Laboratory Press, ISBN 978-0879698911; Methods in Enzymology (Series), Academic Press; Molecular Cloning: A Laboratory Manual (Fourth Edition), 2012, M. R. Green, et al., Cold Spring Harbor Laboratory Press, ISBN 978-1605500560; Bioconjugate Techniques, Third Edition, 2013, G. T. Hermanson, Academic Press, ISBN 978-0123822390; Methods in Plant Biochemistry and Molecular Biology, 1997, W. V. Dashek, CRC Press, ISBN 978-0849394805; Plant Cell Culture Protocols (Methods in Molecular Biology), 2012, V. M. Loyola-Vargas, et al., Humana Press, ISBN 978-1617798177; Plant Transformation Technologies, 2011, C. N. Stewart, et al., Wiley-Blackwell, ISBN 978-0813821955; Recombinant Proteins from Plants (Methods in Biotechnology), 2010, C. Cunningham, et al., Humana Press, ISBN 978-1617370212; Plant Genomics: Methods and Protocols (Methods in Molecular Biology), 2009, D. J. Somers, et al., Humana Press, ISBN 978-1588299970; Plant Biotechnology: Methods in Tissue Culture and Gene Transfer, 2008, R. Keshavachandran, et al., Orient Blackswan, ISBN 978-8173716164.

As used herein and described in detail below, the term “sesPN” refers to a “spacer element sequence polynucleotide” of the present invention and the term “casPN” refers to a “Cas-associated polynucleotide (lacking a spacer element)” (i.e., a Cas protein associated polynucleotide lacking a spacer element) of the present invention.

As used herein, the term “Cas protein” and “CRISPR-Cas protein” refer to CRISPR-associated proteins including, but not limited to Cas9 proteins, Cas9-like proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof. In a preferred embodiment, a Cas protein is a Class 2 CRISPR-associated protein, for example a Class 2 Type II CRISPR-associated protein or a Class 2 Type V CRISPR-associated protein. Each wild-type CRISPR-Cas protein interacts with one or more cognate polynucleotide (most typically RNA) to form a nucleoprotein complex (most typically a ribonucleoprotein complex).

The term “Cas9 protein” as used herein refers to a Cas9 wild-type protein derived from Type II CRISPR-Cas9 systems, modifications of Cas9 proteins, variants of Cas9 proteins, Cas9 orthologs, and combinations thereof. The term “dCas9” as used herein refers to variants of Cas9 protein that are nuclease-deactivated Cas9 proteins, also termed “catalytically inactive Cas9 protein,” or “enzymatically inactive Cas9.”

The term “Cpf1 protein” as used herein refers to a Cpf1 wild-type protein derived from Type V CRISPR-Cpf1 systems, modifications of Cpf1 proteins, variants of Cpf1 proteins, Cpf1 orthologs, and combinations thereof. The term “dCpf1” as used herein refers to variants of Cpf1 protein that are nuclease-deactivated Cpf1 proteins, also termed “catalytically inactive Cpf1 protein,” or “enzymatically inactive Cpf1.”

As used herein, the term “cognate” typically refers to a Cas protein and one or more Cas polynucleotides that are able of forming a nucleoprotein complex capable of site-directed binding to a target nucleic acid complementary to the target nucleic acid binding sequence present in one of the Cas polynucleotides.

As used herein, the terms “wild-type,” “naturally occurring” and “unmodified” are used to mean the typical (or most common) form, appearance, phenotype, or strain existing in nature; for example, the typical form of cells, organisms, characteristics, polynucleotides, proteins, macromolecular complexes, genes, RNAs, DNAs, or genomes as they occur in and can be isolated from a source in nature. The wild-type form, appearance, phenotype, or strain serve as the original parent before an intentional modification. Thus, mutant, variant, engineered, recombinant, and modified forms as used herein are not wild-type forms.

As used herein, the terms “engineered,” “genetically engineered,” “recombinant,” “modified,” and “non-naturally occurring” are interchangeable and indicate intentional human manipulation.

As used herein, the terms “nucleic acid,” “nucleotide sequence,” “oligonucleotide,” and “polynucleotide” are interchangeable. All refer to a polymeric form of nucleotides. The nucleotides may be deoxyribonucleotides (DNA), ribonucleotides (RNA), analogs thereof, or combinations thereof, and may be of any length. Polynucleotides may perform any function and may have any secondary structure and three-dimensional structure. The terms encompass known analogs of natural nucleotides and nucleotides that are modified in the base, sugar and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include methylated nucleotides. Nucleotide structure may be modified before or after a polymer is assembled. Following polymerization, polynucleotides may be additionally modified via, for example, conjugation with a labeling component or target-binding component. A nucleotide sequence may incorporate non-nucleotide components. The terms also encompass nucleic acids comprising modified backbone residues or linkages, that (i) are synthetic, naturally occurring, and non-naturally occurring, and (ii) have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) nucleosides (Exiqon, Inc., Woburn, Mass.), glycol nucleic acid, bridged nucleic acids, and morpholino structures.

Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA.

In phosphorothioate nucleic acids, the phosphorothioate (PS) bond substitutes a sulfur atom for a non-bridging oxygen in the polynucleotide phosphate backbone. This modification makes the internucleotide linkage resistant to nuclease degradation. In some embodiments, phosphorothioate bonds are introduced between the last 3-5 nucleotides at the 5′- or 3′-end of a polynucleotide sequence to inhibit exonuclease degradation. Placement of phosphorothioate bonds throughout an entire oligonucleotide helps reduce degradation by endonucleases as well.

Threose nucleic acid (TNA) is an artificial genetic polymer. The backbone structure of TNA comprises repeating threose sugars linked by phosphodiester bonds. TNA polymers are resistant to nuclease degradation. TNA can self-assemble by base-pair hydrogen bonding into duplex structures.

Linkage inversions can be introduced into polynucleotides through use of “reversed phosphoramidites” (see, e.g., www.ucalgary.ca/dnalab/synthesis/-modifications/linkages). Typically, such polynucleotides have phosphoramidite groups on the 5′-OH position and a dimethoxytrityl (DMT) protecting group on the 3′-OH position. Normally, the DMT protecting group is on the 5′-OH and the phosphoramidite is on the 3′-OH. The most common use of linkage inversion is to add a 3′-3′ linkage to the end of a polynucleotide with a phosphorothioate backbone. The 3′-3′ linkage stabilizes the polynucleotide to exonuclease degradation by creating an oligonucleotide having two 5′-OH ends and no 3′-OH end.

Polynucleotide sequences are displayed herein in the conventional 5′ to 3′ orientation unless otherwise indicated.

As used herein, the term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementary, the two sequences are perfectly complementary, i.e., all of a first polynucleotide's contiguous residues hydrogen bond with the same number of contiguous residues in a second polynucleotide.

As used herein, the term “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, etc.), available through the worldwide web at sites including GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity, as used herein, between two polynucleotides or two polypeptides is typically between about 90% identity and 100% identity, for example, about 90% identity or higher, preferably about 95% identity or higher, more preferably about 98% identity or higher. A moderate degree of sequence identity, as used herein, between two polynucleotides or two polypeptides is typically between about 80% identity to about 85% identity, for example, about 80% identity or higher, preferably about 85% identity. A low degree of sequence identity, as used herein, between two polynucleotides or two polypeptides is typically between about 50% identity and 75% identity, for example, about 50% identity, preferably about 60% identity, more preferably about 75% identity. For example, a Cas protein (e.g., a Cas9 comprising amino acid substitutions, or a Cpf1 comprising amino acid substitutions) can have a moderate degree of sequence identity, or preferably a high degree of sequence identity, over its length to a reference Cas protein (e.g., a wild-type Cas9 or a wild-type Cpf1, respectively). As another example, a casPN (e.g., a casPN that complexes with a Cas9 protein, or a casPN that complexes with a Cpf1 protein) can have a moderate degree of sequence identity, or preferably a high degree of sequence identity, over its length to a reference wild-type polynucleotide that complexes with the reference Cas protein (e.g., a sgRNA that forms site-directed complex with Cas9 or a crRNA that forms site-directed complex with Cpf1).

As used herein, “hybridization” or “hybridize” or “hybridizing” is the process of combining two complementary single-strand DNA or RNA molecules and allowing them to form a single double-stranded molecule (DNA/DNA, DNA/RNA, RNA/RNA) through hydrogen base pairing. Hybridization stringency is typically determined by the hybridization temperature and the salt concentration of the hybridization buffer, for example, high temperature and low salt provide high stringency hybridization conditions. Examples of salt concentration ranges and temperature ranges for different hybridization conditions are as follows: high stringency, approximately 0.01M to approximately 0.05M salt, hybridization temperature 5° C. to 10° C. below Tm; moderate stringency, approximately 0.16M to approximately 0.33M salt, hybridization temperature 20° C. to 29° C. below Tm; low stringency, approximately 0.33M to approximately 0.82M salt, hybridization temperature 40° C. to 48° C. below Tm. Tm of duplex nucleic acids is calculated by standard methods well-known in the art (Maniatis, T., et al (1982) Molecular Cloning: A Laboratory Manual, Cold Spring Harbor Laboratory Press: New York; Casey, J., et al., (1977) Nucleic Acids Res., 4: 1539; Bodkin, D. K., et al., (1985) J. Virol. Methods, 10: 45; Wallace, R. B., et al. (1979) Nucleic Acids Res. 6: 3545.) Algorithm prediction tools to estimate Tm are also widely available. High stringency conditions for hybridization typically refer to conditions under which a nucleic acid having complementarity to a target sequence predominantly hybridizes with the target sequence, and substantially does not hybridize to non-target sequences. Typically, hybridization conditions are of moderate stringency, preferably high stringency.

As used herein, a “stem-loop structure” or “stem-loop element” refers to a polynucleotide having a secondary structure that includes a region of nucleotides that are known or predicted to form a double-stranded region (the “stem element”) that is linked on one side by a region of predominantly single-strand nucleotides (the “loop element”). The term “hairpin” element is also used herein to refer to stem-loop structures. Such structures are well known in the art. The base pairing may be exact. However, as is known in the art, that a stem element does not require exact base pairing. Thus, the stem element may include one or more base mismatches or non-paired bases.

As used herein, the term “recombination” refers to a process of exchange of genetic information between two polynucleotides.

As used herein, the term “homology-directed repair (HDR)” refers to DNA repair that takes place in cells, for example, during repair of double-strand breaks in DNA. HDR requires nucleotide sequence homology and uses a “donor template” (e.g., donor template DNA) or oligonucleotide to repair the sequence wherein the double-strand break occurred (e.g., DNA target sequence). Donor template and “donor polynucleotide” are used interchangeably herein. HDR results in the transfer of genetic information from, for example, the donor template DNA to the DNA target sequence. HDR may result in alteration of the DNA target sequence (e.g., insertion, deletion, mutation) if the donor template DNA sequence or oligonucleotide sequence differs from the DNA target sequence and part or all of the donor template DNA polynucleotide or oligonucleotide is incorporated into the DNA target sequence. In some embodiments, an entire donor template DNA polynucleotide, a portion of the donor template DNA polynucleotide, or a copy of the donor polynucleotide is integrated at the site of the DNA target sequence.

As used herein, the term “non-homologous end joining (NHEJ)” refers to the repair of double-strand breaks in DNA by direct ligation of one end of the break to the other end of the break without a requirement for a donor template DNA. NHEJ in the absence of a donor template DNA often results in a small number of nucleotides randomly inserted or deleted (“indel” or “indels”) at the site of the double-strand break.

The terms “vector” and “plasmid” as used herein refer to a polynucleotide vehicle to introduce genetic material into a cell. Vectors can be linear or circular. Vectors can integrate into a target genome of a host cell or replicate independently in a host cell. The four major types of vectors are plasmids, viral vectors, cosmids, and artificial chromosomes. Typically, vectors comprise an origin of replication, a multicloning site, and/or a selectable marker. An expression vector typically comprises an expression cassette.

As used, herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in a vector to form an expression vector.

As used, herein a “targeting vector” is a recombinant DNA construct typically comprising tailored DNA arms homologous to genomic DNA that flanks critical elements of a target gene or target sequence. When introduced into a cell the targeting vector integrates into the cell genome via homologous recombination. Elements of the target gene can be modified in a number of ways including deletions and/or insertions. A defective target gene can be replaced by a functional target gene, or in the alternative a functional gene can be knocked out. Optionally a targeting vector comprises a selection cassette comprising a selectable marker that is introduced into the target gene. Targeting regions adjacent or sometimes within a target gene can be used to affect regulation of gene expression.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like.

As used herein, the term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding sequence. Operably linked DNA sequences encoding regulatory sequences are typically contiguous to the coding sequence. However, enhancers can function when separated from a promoter by up to several kilobases or more. Accordingly, some polynucleotide elements may be operably linked but not contiguous.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

As used herein, the term “modulate” refers to a change in the quantity, degree or amount of a function. For example, the sesPN/casPN/Cas protein systems disclosed herein may modulate the activity of a promoter sequence by binding at or near the promoter. Depending on the action occurring after binding, the sesPN/casPN/Cas protein systems can induce, enhance, suppress, or inhibit transcription of a gene operatively linked to the promoter sequence. Thus, “modulation” of gene expression includes both gene activation and gene repression.

Modulation can be assayed by determining any characteristic directly or indirectly affected by the expression of the target gene. Such characteristics include, e.g., changes in RNA or protein levels, protein activity, product levels, associated gene expression, or activity level of reporter genes. Accordingly, the terms “modulating expression,” “inhibiting expression,” and “activating expression” of a gene can refer to the ability of a sesPN/casPN/Cas protein system to change, activate, or inhibit transcription of a gene.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms may be used to refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation.

Polypeptides and polynucleotides can be made using routine techniques in the field of molecular biology (see, e.g., standard texts discussed above). Furthermore, essentially any polypeptide or polynucleotide can be custom ordered from commercial sources.

The terms “fusion protein” and “chimeric protein” as used herein refer to a single protein created by joining two or more proteins, protein domains, or protein fragments that do not naturally occur together in a single protein. For example, a fusion protein can contain a first domain from a Cas9 or Cpf1 protein and a second domain from a protein other than Cas9 or Cpf1. The modification to include such domains in fusion protein may confer additional activity on the modified site-directed polypeptides. Such activities can include nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity) that modifies a polypeptide associated with target nucleic acid (e.g., a histone). A fusion protein can also comprise epitope tags (e.g., histidine tags, FLAG® (Sigma Aldrich, St. Louis, Mo.) tags, Myc tags), reporter protein sequences (e.g., glutathione-S-transferase, beta-galactosidase, luciferase, green fluorescent protein, cyan fluorescent protein, yellow fluorescent protein), nucleic acid binding domains (e.g., a DNA binding domain, an RNA binding domain). In some embodiments, linker sequences are used to connect the two or more proteins, protein domains, or protein fragments.

The term “binding” as used herein refers to a non-covalent interaction between macromolecules (e.g., between a protein and a polynucleotide, between a polynucleotide and a polynucleotide, and between a protein and a protein). Such non-covalent interaction is also referred to as “associating” or “interacting” (e.g., when a first macromolecule interacts with a second macromolecule, the first macromolecule binds to second macromolecule in a non-covalent manner). Some portions of a binding interaction may be sequence-specific; however, all components of a binding interaction do not need to be sequence-specific, such as a protein's contacts with phosphate residues in a DNA backbone. Binding interactions can be characterized by a dissociation constant (Kd). “Affinity” refers to the strength of binding. An increased binding affinity is correlated with a lower Kd. An example of non-covalent binding is hydrogen bond formation between base pairs.

As used herein, the term “isolated” can refer to a nucleic acid or polypeptide that, by the hand of a human, exists apart from its native environment and is therefore not a product of nature. Isolated means substantially pure. An isolated nucleic acid or polypeptide can exist in a purified form and/or can exist in a non-native environment such as, for example, in a recombinant cell.

As used herein, a “host cell” generally refers to a biological cell. A cell can be the basic structural, functional and/or biological unit of a living organism. A cell can originate from any organism having one or more cells. Examples of host cells include, but are not limited to: a prokaryotic cell, a eukaryotic cell, a bacterial cell, an archaeal cell, a cell of a single-cell eukaryotic organism, a protozoa cell, a cell from a plant (e.g. cells from plant crops (such as soy, tomatoes, sugar beets, pumpkin, hay, cannabis, tobacco, plantains, yams, sweet potatoes, cassava, potatoes, wheat, sorghum, soybean, rice, wheat, corn, oil-producing Brassica (e.g., oil-producing rapeseed and canola), cotton, sugar cane, sunflower, millet, and alfalfa), fruits, vegetables, grains, seeds, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.). Furthermore, a cell can be a stem cell or progenitor cell.

The term “subject” as used herein refers to any member of the subphylum chordata, including, without limitation, humans and other primates, including non-human primates such as rhesus macaque, chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese; and the like. The term does not denote a particular age. Thus, adult, young, and newborn individuals are intended to be covered. In some embodiments, a host cell is derived from a subject (e.g., stem cells, progenitor cells, tissue specific cells). In some embodiments the “subject is a non-human subject.”

As used herein, the term “transgenic organism” refers to an organism comprising a recombinantly introduced polynucleotide.

As used herein, the terms “transgenic plant cell” and “transgenic plant” are interchangeable and refer to a plant cell or a plant containing a recombinantly introduced polynucleotide. Included in the term transgenic plant is the progeny (any generation) of a transgenic plant or a seed such that the progeny or seed comprises a DNA sequence encoding a recombinantly introduced polynucleotide or a fragment thereof.

As used herein, the phrase “generating a transgenic plant cell or a plant” refers to using recombinant DNA methods and techniques to construct a vector for plant transformation to transform the plant cell or the plant and to generate the transgenic plant cell or the transgenic plant.

CRISPR-Cas systems have recently been reclassified into two classes, comprising five types and sixteen subtypes (Makarova, K., et al., Nature Reviews Microbiology 13:1-15 (2015)). This classification is based upon identifying all cas genes in a CRISPR-Cas locus and then determining the signature genes in each CRISPR-Cas locus, ultimately determining that the CRISPR-Cas systems can be placed in either Class 1 or Class 2 based upon the genes encoding the effector module, i.e., the proteins involved in the interference stage. Recently a sixth CRISPR-Cas system has been identified (Abudayyeh O., et al. “C2c2 is a single-component programmable RNA-guided RNA-targeting CRISPR effector,” Science 2016 Jun. 2, pii: aaf5573 [Epub]).

Class 1 systems have a multi-subunit crRNA-effector complex, whereas Class 2 systems have a single protein, such as Cas9, Cpf1, C2c1, C2c2, C2c3, or a crRNA-effector complex. Class 1 systems comprise Type I, Type III and Type IV systems. Class 2 systems comprise Type II and Type V systems.

Type I systems all have a Cas3 protein that has helicase activity and cleavage activity. Type I systems are further divided into seven sub-types (I-A to I-F and I-U). Each type I subtype has a defined combination of signature genes and distinct features of operon organization. For example, sub-types I-A and I-B appear to have the cas genes organized in two or more operons, whereas sub-types I-C through I-F appear to have the cas genes encoded by a single operon. Type I systems have a multiprotein crRNA-effector complex that is involved in the processing and interference stages of the CRISPR-Cas immune system. This multiprotein complex is known as CRISPR-associated complex for antiviral defense (Cascade). Sub-type I-A comprises csa5 which encodes a small subunit protein and a cas8 gene that is split into two, encoding degraded large and small subunits and also has a split cas3 gene. An example of an organism with a sub-type I-A CRISPR-Cas system is Archaeoglobus fulgidus.

Sub-type I-B has a cas1-cas2-cas3-cas4-cas5-cas6-cas7-cas8 gene arrangement and lacks a csa5 gene. An example of an organism with sub-type I-B is Clostridium kluyveri. Sub-type I-C does not have a cas6 gene. An example of an organism with sub-type I-C is Bacillus halodurans. Sub-type I-D has a Cas10d instead of a Cas8. An example of an organism with sub-type I-D is Cyanothece spp. Sub-type I-E does not have a cas4. An example of an organism with sub-type I-E is Escherichia coli. Sub-type I-F does not have a cas4 and has a cas2 fused to a cas3. An example of an organism with sub-type I-F is Yersinia pseudotuberculosis. An example of an organism with sub-type I-U is Geobacter sulfurreducens.

All type III systems possess a cas10 gene, which encodes a multidomain protein containing a Palm domain (a variant of the RNA recognition motif (RRM)) that is homologous to the core domain of numerous nucleic acid polymerases and cyclases and that is the largest subunit of type III crRNA-effector complexes. All type III loci also encode the small subunit protein, one Cas5 protein and typically several Cas7 proteins. Type III can be further divided into four sub-types, III-A through III-D. Sub-type III-A has a csm2 gene encoding a small subunit and also has cas1, cas2 and cas6 genes. An example of an organism with sub-type III-A is Staphylococcus epidermidis. Sub-type III-B has a cmr5 gene encoding a small subunit and also typically lacks cas1, cas2 and cas6 genes. An example of an organism with sub-type III-B is Pyrococcus furiosus. Sub-type III-C has a Cas10 protein with an inactive cyclase-like domain and lacks a cas1 and cas2 gene. An example of an organism with sub-type III-C is Methanothermobacter thermautotrophicus. Sub-type III-D has a Cas10 protein that lacks the HD domain, it lacks a cas1 and cas2 gene and has a cas5-like gene known as csx10. An example of an organism with sub-type III-D is Roseiflexus spp.

Type IV systems encode a minimal multisubunit crRNA-effector complex comprising a partially degraded large subunit, Csf1, Cas5, Cas7, and in some cases, a putative small subunit. Type IV systems lack cas1 and cas2 genes. Type IV systems do not have sub-types, but there are two distinct variants. One Type IV variant has a DinG family helicase, whereas a second type IV variant lacks a DinG family helicase, but has a gene encoding a small α-helical protein. An example of an organism with a Type IV system is Acidithiobacillus ferrooxidans.

Type II systems have cas1, cas2 and cas9 genes. cas9 encodes a multidomain protein that combines the functions of the crRNA-effector complex with target DNA cleavage. Type II systems also encode a tracrRNA. Type II systems are further divided into three sub-types, sub-types II-A, II-B and II-C. Sub-type II-A contains an additional gene, csn2. An example of an organism with a sub-type II-A system is Streptococcus thermophilus. Sub-type II-B lacks csn2, but has cas4. An example of an organism with a sub-type II-B system is Legionella pneumophila. Sub-type II-C is the most common Type II system found in bacteria and has only three proteins, Cas1, Cas2 and Cas9. An example of an organism with a sub-type II-C system is Neisseria lactamica.

Type V systems have a cpf1 gene and cas1 and cas2 genes. The cpf1 gene encodes a protein, Cpf1, that has a RuvC-like nuclease domain that is homologous to the respective domain of Cas9, but lacks the HNH nuclease domain that is present in Cas9 proteins. Type V systems have been identified in several bacteria, including Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA 33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237(MbCpf1), Smithella spp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1). Recently it has been demonstrated that Cpf1 also has RNase activity and it is responsible for pre-crRNA processing (Fonfara, I., et al., “The CRISPR-associated DNA-cleaving enzyme Cpf1 also processes precursor CRISPR RNA,” Nature 28; 532(7600):517-21 (2016)).

In Class 1 systems, the expression and interference stages involve multisubunit CRISPR RNA (crRNA)-effector complexes. In Class 2 systems, the expression and interference stages involve a single large protein, e.g., Cas9, Cpf1, C2c1, C2c1, or C2c3.

In Class 1 systems, pre-crRNA is bound to the multisubunit crRNA-effector complex and processed into a mature crRNA. In Type I and III systems this involves an RNA endonuclease, e.g., Cas6. In Class 2 Type II systems, pre-crRNA is bound to Cas9 and processed into a mature crRNA in a step that involves RNase III and a tracrRNA. However, in at least one Type II CRISPR-Cas system, that of Neisseria meningitidis, crRNAs with mature 5′ ends are directly transcribed from internal promoters, and crRNA processing does not occur.

In Class 1 systems the crRNA is associated with the crRNA-effector complex and achieves interference by combining nuclease activity with RNA-binding domains and base pair formation between the crRNA and a target nucleic acid.

In Type I systems, the crRNA and target binding of the crRNA-effector complex involves Cas7, Cas5, and Cas8 fused to a small subunit protein. The target nucleic acid cleavage of Type I systems involves the HD nuclease domain, which is either fused to the superfamily 2 helicase Cas3′ or is encoded by a separate gene, cas3.

In Type III systems, the crRNA and target binding of the crRNA-effector complex involves Cas7, Cas5, Cas10 and a small subunit protein. The target nucleic acid cleavage of Type III systems involves the combined action of the Cas7 and Cas10 proteins, with a distinct HD nuclease domain fused to Cas10, which is thought to cleave single-strand DNA during interference.

In Class 2 systems the crRNA is associated with a single protein and achieves interference by combining nuclease activity with RNA-binding domains and base pair formation between the crRNA and a target nucleic acid.

In Type II systems, the crRNA and target binding involves Cas9 as does the target nucleic acid cleavage. In Type II systems, the RuvC-like nuclease (RNase H fold) domain and the HNH (McrA-like) nuclease domain of Cas9 each cleave one of the strands of the target nucleic acid. The Cas9 cleavage activity of Type II systems also requires hybridization of crRNA to tracrRNA to form a duplex that facilitates the crRNA and target binding by the Cas9.

In Type V systems, the crRNA and target binding involves Cpf1 as does the target nucleic acid cleavage. In Type V systems, the RuvC-like nuclease domain of Cpf1 cleaves one strand of the target nucleic acid and a putative nuclease domain cleaves the other strand of the target nucleic acid in a staggered configuration, producing 5′ overhangs, which is in contrast to the blunt ends generated by Cas9 cleavage. These 5′ overhangs may facilitate insertion of DNA through non-homologous end-joining methods.

The Cpf1 cleavage activity of Type V systems also does not require hybridization of crRNA to tracrRNA to form a duplex, rather the crRNA of Type V systems use a single crRNA that has a stem loop structure forming an internal duplex. Cpf1 binds the crRNA in a sequence and structure specific manner, that recognizes the stem loop and sequences adjacent to the stem loop, most notably, the nucleotide 5′ of the spacer sequences that hybridizes to the target nucleic acid. This stem loop structure is typically in the range of 15 to 19 nucleotides in length. Substitutions that disrupt this stem loop duplex abolish cleavage activity, whereas other substitutions that do not disrupt the stem loop duplex do not abolish cleavage activity. In Type V systems, the crRNA forms a stem loop structure at the 5′ end and the sequence at the 3′ end is complementary to a sequence in a target nucleic acid.

Other proteins associated with Type V crRNA and target binding and cleavage include Class 2 candidate 1 (C2c1) and Class 2 candidate 3 (C2c3). C2c1 and C2c3 proteins are similar in length to Cas9 and Cpf1 proteins, ranging from approximately 1,100 amino acids to approximately 1,500 amino acids. C2c1 and C2c3 proteins also contain RuvC-like nuclease domains and have an architecture similar to Cpf1. C2c1 proteins are similar to Cas9 proteins in requiring a crRNA and a tracrRNA for target binding and cleavage, but have an optimal cleavage temperature of 50° C. C2c1 proteins target an AT-rich PAM, which similar to Cpf1, is 5′ of the target sequence (see, e.g., Shmakov, S., et al. Molecular Cell 60(3):385-397 (2015)).

Class 2 candidate 2 (C2c2) does not share sequence similarity to other CRISPR effector proteins, and was recently identified as a Type VI system (Abudayyeh O., et al., “C2c2 is a single-component programmable RNA-guided RNA-targeting CRISPR effector,” Science 2016 Jun. 2, pii: aaf5573 [Epub]). C2c2 proteins have two HEPN domains and demonstrate ssRNA-cleavage activity. C2c2 proteins are similar to Cpf1 proteins in requiring a crRNA for target binding and cleavage, while not requiring tracrRNA. Also like Cpf1, the crRNA for C2c2 proteins forms a stable hairpin, or stem loop structure, that aid in association with the C2c2 protein.

Regarding Class 2 Type II CRISPR Cas systems, a large number of Cas9 orthologs are known in the art as well as their associated polynucleotide components (tracrRNA and crRNA) (see, e.g., “Supplementary Table S2. List of bacterial strains with identified Cas9 orthologs,” Fonfara, Ines, et al., “Phylogeny of Cas9 Determines Functional Exchangeability of Dual-RNA and Cas9 among Orthologous Type II CRISPR/Cas Systems,” Nucleic Acids Research 42.4 (2014): 2577-2590, including all Supplemental Data; Chylinski K., et al., “Classification and evolution of type II CRISPR-Cas systems,” Nucleic Acids Research, 2014; 42(10):6091-6105, including all Supplemental Data.).

In addition, Cas9-like synthetic proteins are known in the art (see U.S. Published Patent Application No. 2014-0315985, published 23 Oct. 2014). Aspects of the present invention can be practiced by one of ordinary skill in the art following the guidance of the specification to use Type II CRISPR Cas proteins and Cas-protein encoding polynucleotides, including, but not limited to Cas9, Cas9-like, proteins encoded by Cas9 orthologs, Cas9-like synthetic proteins, and variants and modifications thereof. The cognate RNA components of these Cas proteins can be manipulated and modified for use in the practice of the present invention by one of ordinary skill in the art following the guidance of the present specification.

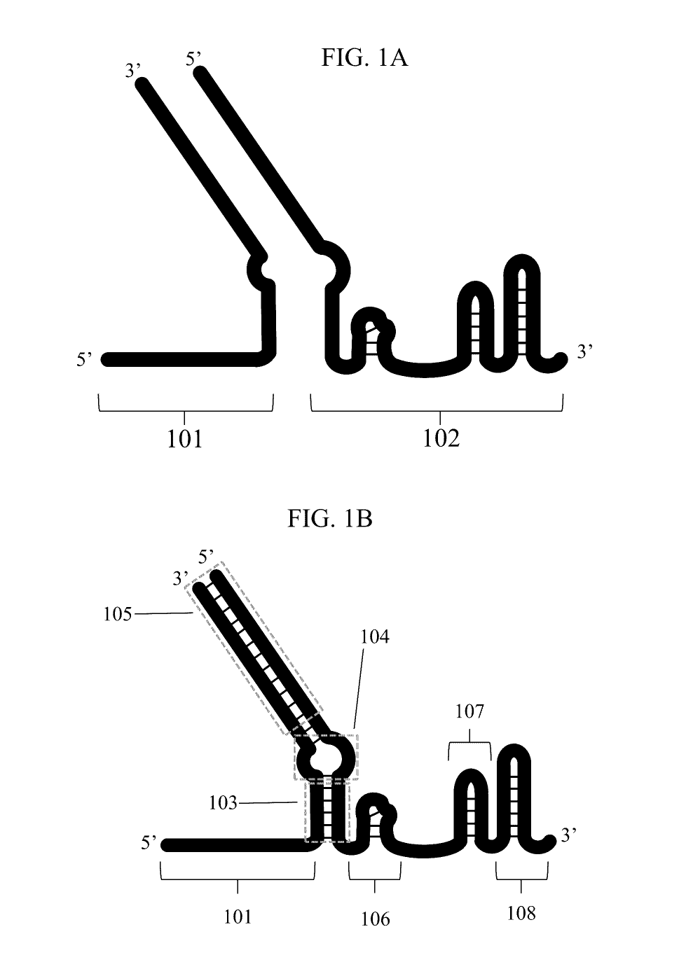

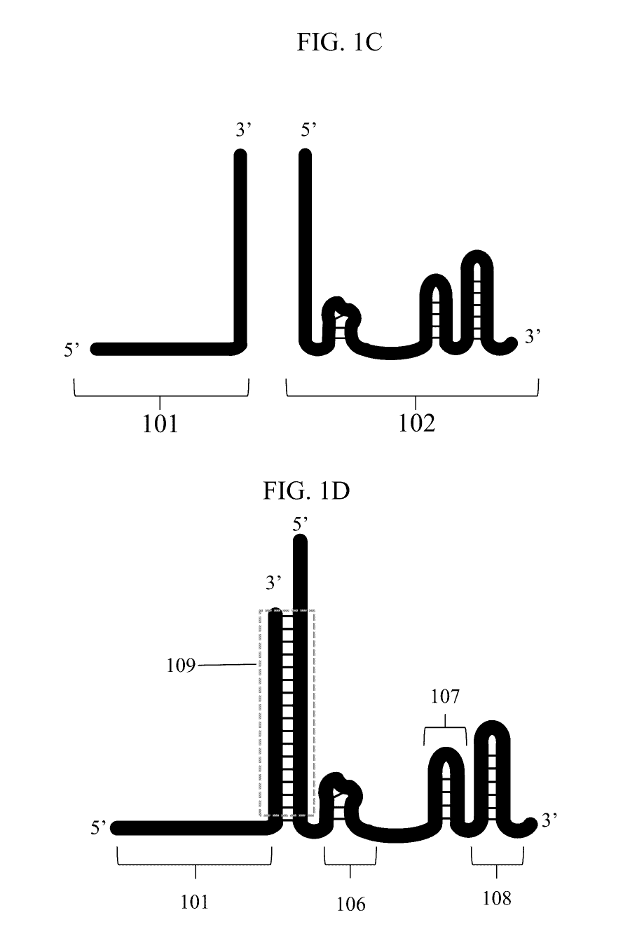

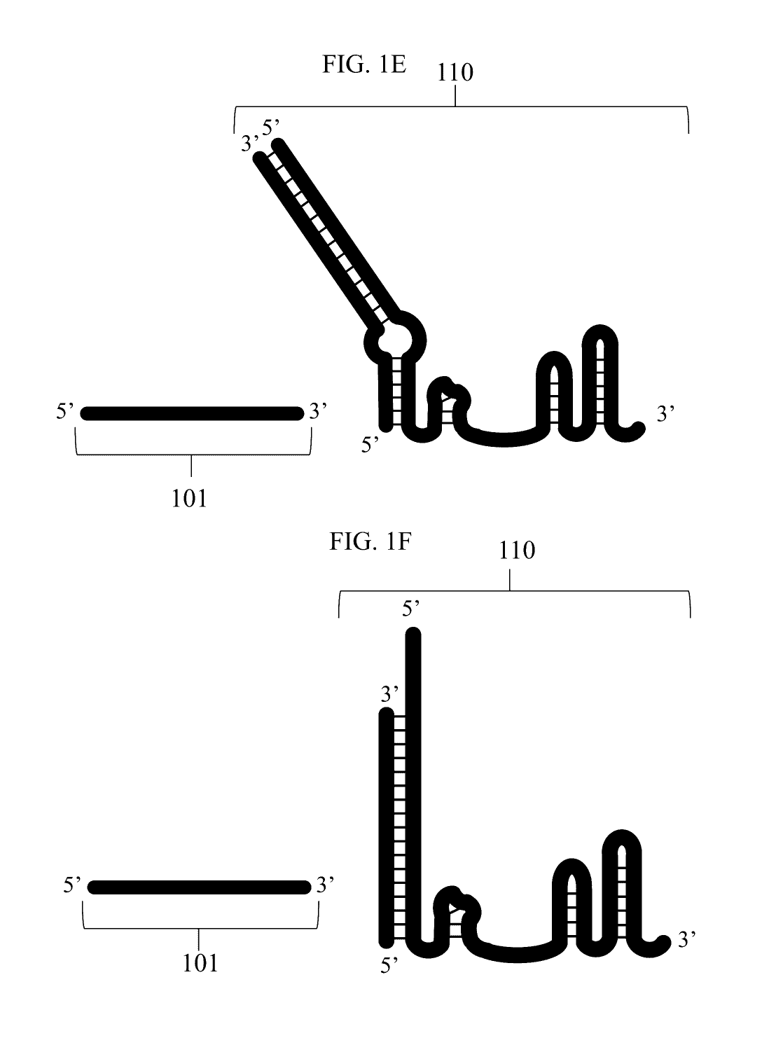

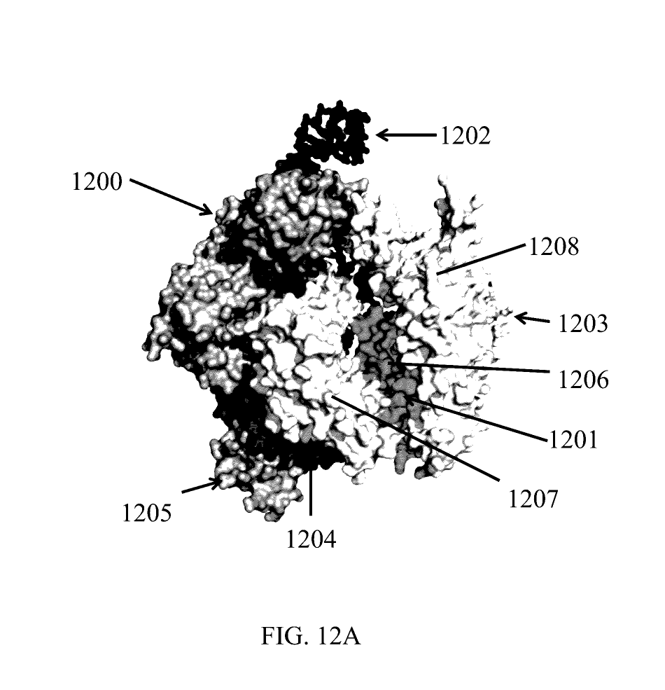

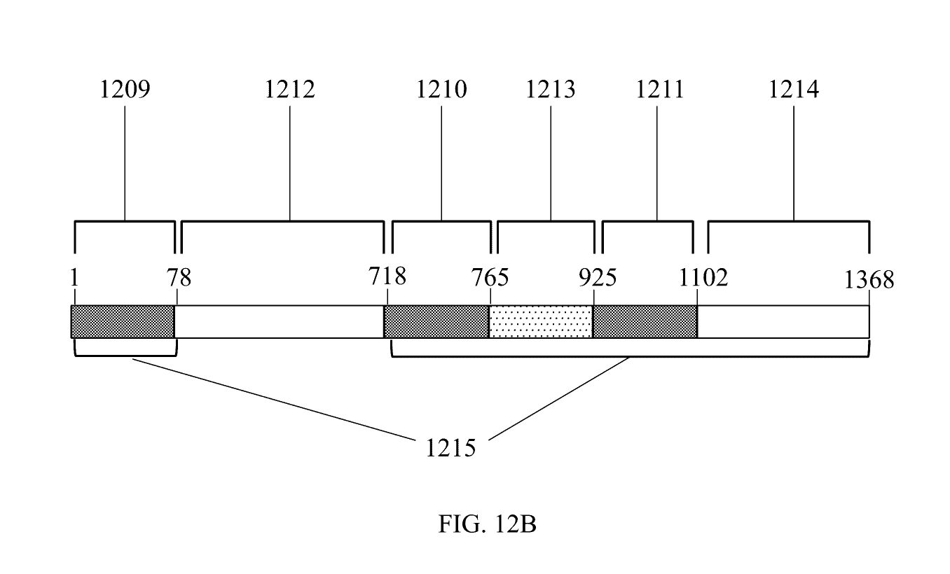

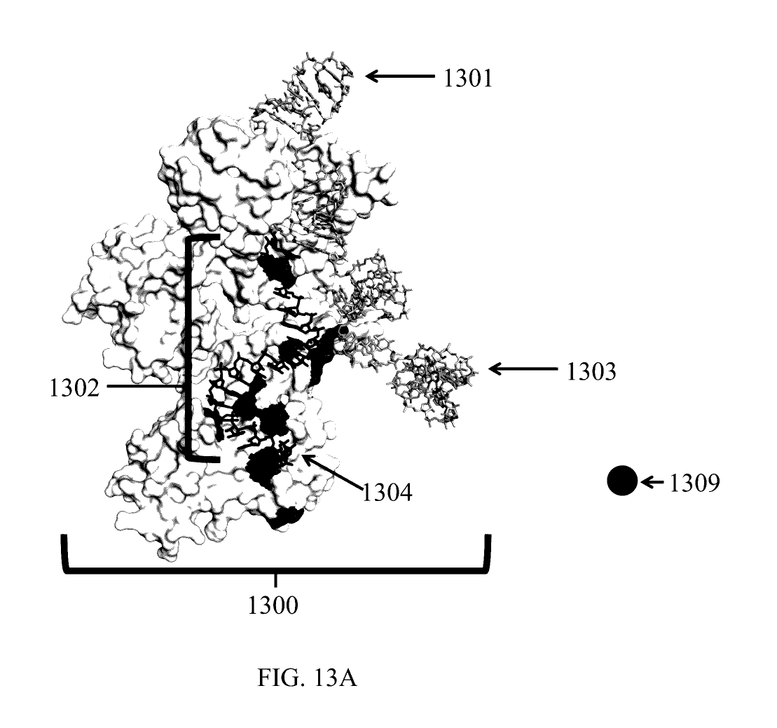

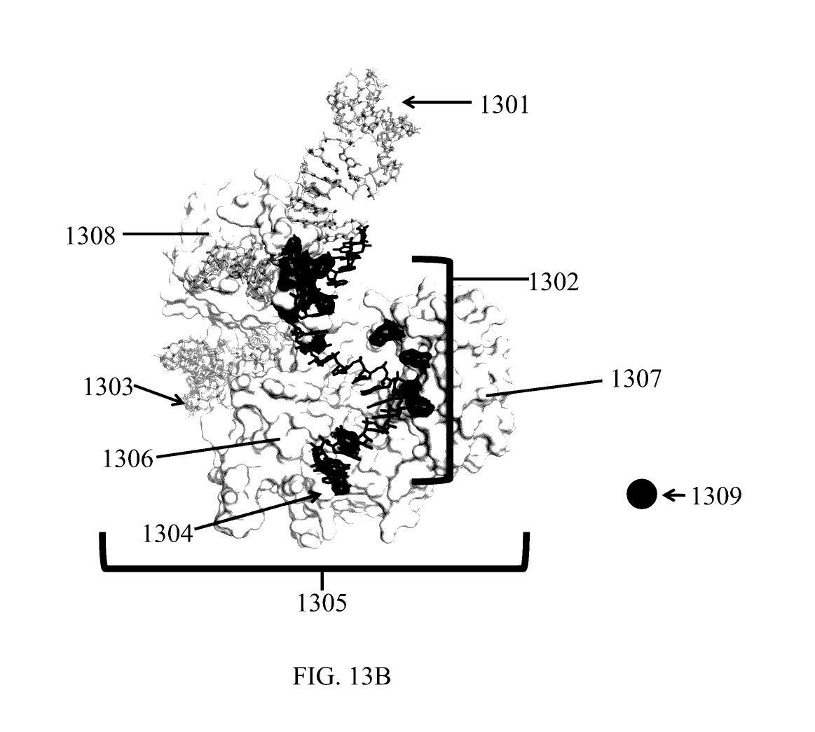

Cas9 is an exemplary Type II CRISPR Cas protein. Cas9 is an endonuclease that can be programmed by the tracrRNA/crRNA to cleave, site-specifically, target DNA using two distinct endonuclease domains (HNH and RuvC/RNase H-like domains) (see U.S. Published Patent Application No. 2014-0068797, published 6 Mar. 2014; see also Jinek M., et al., “A programmable dual-RNA-guided DNA endonuclease in adaptive bacterial immunity,” Science, 2012; 337:816-21;). FIG. 12B presents a model of the domain arrangement of SpyCas9 relative to its primary sequence structure. Two RNA components of a Type II CRISPR Cas system are illustrated in FIG. 1A and FIG. 1C. Typically, each wild-type Type II CRISPR Cas system comprises a tracrRNA and a crRNA.

The crRNA has a region of complementarity to a potential DNA target sequence (FIG. 1B, 101; FIG. 1D, 101) and a second region that forms base-pair hydrogen bonds with the tracrRNA to form a secondary structure, typically to form at least a stem structure (FIG. 1B, 103, 104, 105; FIG. 1D, 109). The region of complementarity to the target DNA is the spacer. In some embodiments, the tracrRNA and a crRNA interact through a number of base-pair hydrogen bonds to form secondary RNA structures, for example, as illustrated in FIG. 1B, 103, 104, 105, and FIG. 1D, 109.

The formation of a complex between tracrRNA/crRNA and Cas9 protein results in conformational change of the Cas9 protein that facilitates binding to DNA, endonuclease activities of the Cas9 protein, and crRNA-guided site-specific DNA cleavage by the endonuclease. For a Cas9 protein/tracrRNA/crRNA ribonucleoprotein complex to cleave a DNA target sequence, the DNA target sequence is adjacent to a protospacer adjacent motif (PAM) associated with the Cas9 protein/tracrRNA/crRNA ribonucleoprotein complex.

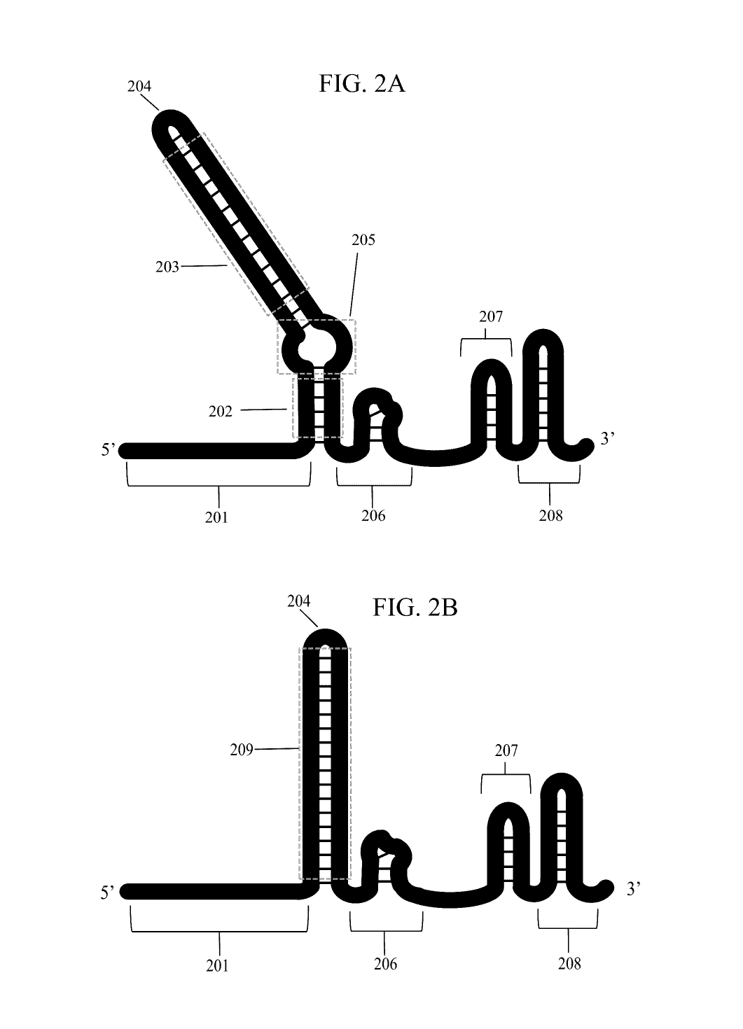

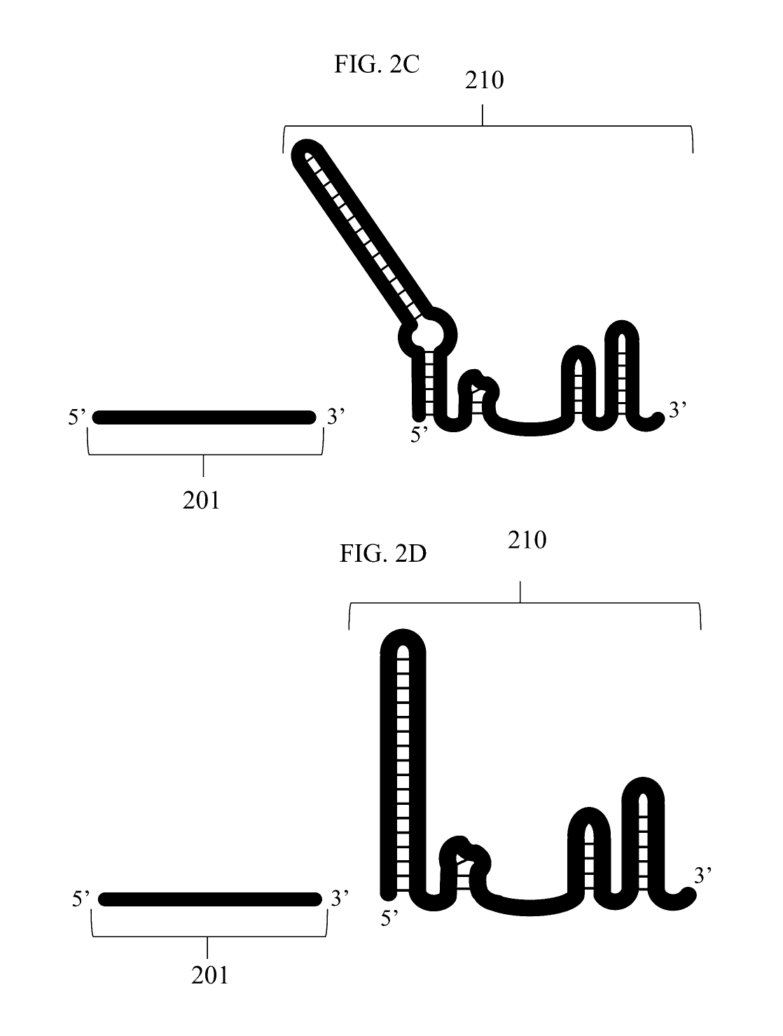

The term sgRNA typically refers to a single guide RNA (i.e., a single, contiguous polynucleotide sequence). In Class 2 Type II CRISPR Cas systems, a sgRNA essentially comprises a crRNA connected at its 3′ end to the 5′ end of a tracrRNA through a “loop” sequence (see, e.g., U.S. Published Patent Application No. 2014-0068797, published 6 Mar. 2014). sgRNA interacts with a cognate Cas protein essentially as described for tracrRNA/crRNA polynucleotides, as discussed above. Similar to crRNA, sgRNA has a spacer, a region of complementarity to a potential DNA target sequence (FIG. 2A, 201), adjacent a second region that forms base-pair hydrogen bonds that form a secondary structure, typically a stem structure.

FIG. 12A provides a three-dimensional model based on the crystal structure of Streptococcus pyogenes Cas9 (SpyCas9) in an active complex with sgRNA. The relationship of the sgRNA to the helical domain and the catalytic domain is illustrated. The 3′ and 5′ ends of the sgRNA are indicated, as well as exposed portions of the sgRNA. The spacer RNA of the sgRNA is not visible because it is surrounded by the alpha-helical lobe (helical domain) and the catalytic nuclease lobe (catalytic domain). The spacer RNA of the sgRNA is located in the 5′ end region of the sgRNA. The RuvC and HNH nuclease domains, when active, each cut a different DNA strand in target DNA. The C-terminal domain (CTD) is involved in recognition of protospacer adjacent motifs (PAMs) in target DNA.

Using a sgRNA/Cas9 protein system (see U.S. Published Patent Application No. 2014-0315985, published 23 Oct. 2014; and later published Briner, A. E., et al., “Guide RNA Functional Modules Direct Cas9 Activity and Orthogonality,” Molecular Cell Volume 56, Issue 2, 23 Oct. 2014, pages 333-339)), it was demonstrated that expendable features can be removed to generate functional miniature sgRNAs. These publications also identify an essential and conserved module, the “nexus,” which is located in the portion of sgRNA that corresponds to tracrRNA (not crRNA). The nexus confers the binding of a sgRNA or a tracrRNA to its cognate Cas9 protein and confers an apoenzyme to haloenzyme conformational transition.

The nexus is located immediately downstream of (i.e., located in the 3′ direction from) the lower stem in Type II CRISPR Cas systems. An example of the relative location of the nexus is illustrated in the sgRNA shown in FIG. 2. U.S. Published Patent Application No. 2014-0315985 and Briner, et al., also disclose consensus sequences and secondary structures of predicted sgRNAs for several sgRNA/Cas9 families. The general arrangement of secondary structures in the predicted sgRNAs up to and including the nexus are presented in FIG. 2A and FIG. 2B herein. FIG. 2A and FIG. 2B presents an overview of and nomenclature for elements of an sgRNA of the Streptococcus pyogenes Cas9. Relative to FIGS. 2A and 2B, there is variation in the number and arrangement of stem structures located 3′ of the nexus in the sgRNAs of U.S. Published Patent Application No. 2014-0315985 and Briner, et al.

Ran, F. A., et al., (“In vivo genome editing using Staphylococcus aureus Cas9,” Nature, 2015, Apr. 9; 520(7546):186-91, including all extended data) present the crRNA/tracrRNA sequences and secondary structures of eight Type II CRISPR Cas systems (see Extended Data FIG. 1 of Ran, F. A., et al.). Predicted tracrRNA structures were based on the Constraint Generation RNA folding model (Zuker, M., “Mfold web server for nucleic acid folding and hybridization prediction,” Nucleic Acids Res., 31, 3406-3415 (2003)). Furthermore, Fonfara, et al., (“Phylogeny of Cas9 Determines Functional Exchangeability of Dual-RNA and Cas9 among Orthologous Type II CRISPR/Cas Systems,” Nucleic Acids Research 42.4 (2014): 2577-2590, including all Supplemental Data, in particular Supplemental Figure S11) present the crRNA/tracrRNA sequences and secondary structures of eight Type II CRISPR-Cas systems. RNA duplex secondary structures were predicted using RNAcofold of the Vienna RNA package (Bernhart, S. H., et al., (2006) “Partition function and base pairing probabilities of RNA heterodimers,” Algorithms Mol. Biol., 1, 3; Hofacker, I. L., et al., (2002) “Secondary structure prediction for aligned RNA sequences. J. Mol. Biol., 319, 1059-1066) and RNAhybrid (bibiserv.techfak.uni-bielefeld.de/mahybrid/)). The structure predictions were then visualized using VARNA (Darty, K., et al., (2009) VARNA: Interactive drawing and editing of the RNA secondary structure Bioinformatics, 25, 1974-1975). Fonfara, et al., show that the crRNA/tracrRNA complex for Campylobacter jejuni does not have the bulge region (illustrated in FIG. 2B herein); however, it retains a stem structure located 3′ of the spacer that is followed in the 3′ direction with another stem structure. With the addition of a loop sequence to connect each crRNA to tracrRNA (3′ end of crRNA to 5′ end of tracr to form a sgRNA), the resulting sgRNAs have at least a stem structure located 3′ of the spacer followed in the 3′ direction with another stem structure corresponding to the position of the nexus as presented in FIG. 2B.

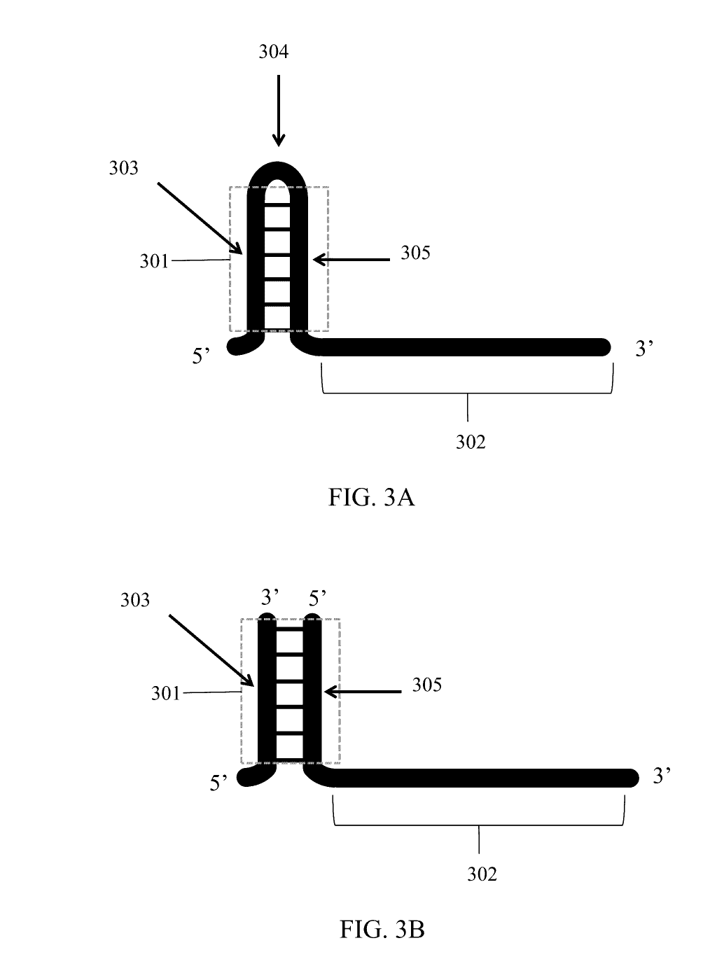

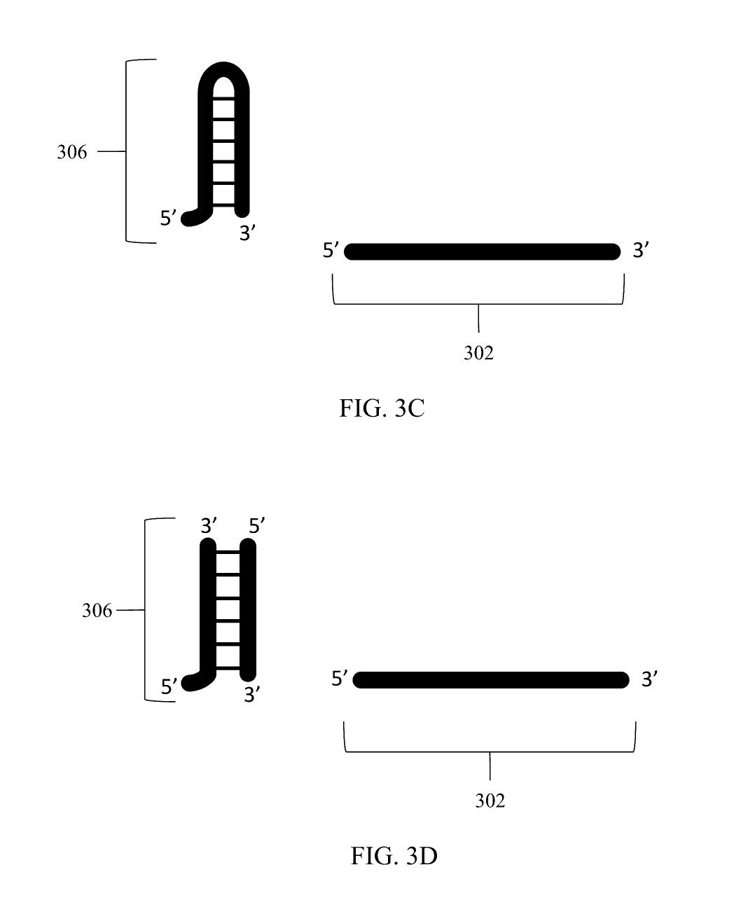

Naturally occurring Type V CRISPR Cas systems, unlike Type II CRISPR Cas systems, do not require a tracrRNA for crRNA maturation and cleavage of a target nucleic acid. FIG. 3A shows a typical structure of a crRNA from a Type V CRISPR system, wherein the DNA target-binding sequence is downstream of a specific secondary structure (i.e., a stem loop structure) that interacts with the Cpf1 protein. The bases 5′ of the stem-loop adopt a pseudoknot structure further stabilizing the stem-loop structure with non-canonical Watson-Crick base pairing (e.g. U base pairs with U) and a triplex interaction involving reverse Hoogsteen base pairing (e.g. U base pairs with A base pairs with U). FIG. 3B illustrates a modification of the Cpf1 polynucleotide stem loop structure.

To date two Type V CRISPR Cas systems, Acidaminococcus and the other from Lachnospiraceae, have demonstrated genome-editing activity in human cells (Zetsche, Bernd, et al., “Cpf1 Is a Single RNA-Guided Endonuclease of a Class 2 CRISPR-Cas System,” Cell 163:759-771 (2015)).

The spacer of Class 2 CRISPR-Cas systems (e.g., FIG. 1B, 101; FIG. 1D, 101; FIG. 2A, 201; FIG. 2B, 201; FIG. 3A, 302; FIG. 3B, 302) can hybridize to a target nucleic acid that is located 5′ or 3′ of a protospacer adjacent motif (PAM), depending upon the Cas protein to be used. A PAM can vary depending upon the site-directed polypeptide to be used. For example, when using the Cas9 from S. pyogenes, the PAM can be a sequence in the target nucleic acid that comprises the sequence 5′-NRR-3′, wherein R can be either A or G, wherein N is any nucleotide, and N is immediately 3′ of the target nucleic acid sequence targeted by the targeting region sequence. A Cas protein may be modified such that a PAM may be different compared to a PAM for an unmodified Cas protein. For example, when using Cas9 protein from S. pyogenes, the Cas9 protein may be modified such that the PAM no longer comprises the sequence 5′-NRR-3′, but instead comprises the sequence 5′-NNR-3′, wherein R can be either A or G, wherein N is any nucleotide, and N is immediately 3′ of the target nucleic acid sequence targeted by the targeting region sequence. Other Cas proteins recognize other PAMs and one of skill in the art is able to determine the PAM for any particular Cas protein. For example, Cpf1 from Francisella novicida was identified as having a 5′-TTN-3′ PAM (Zetsche, et al., Cell; 163(3):759-71 (2015)), but this was unable to support site specific cleavage of a target nucleic acid in vivo. Given the similarity in the guide sequence between Francisella novicida and other Cpf1 proteins, such as the Cpf1 from Acidaminocccus spp. BV3L6, which utilize a 5′-TTTN-3′ PAM, it is more likely that the Francisella novicida Cpf1 protein recognizes and cleaves a site on a target nucleic acid proximal to a 5′-TTTN-3′ PAM with greater specificity and activity than a site on a target nucleic acid proximal to the truncated 5′-TTN-3′ PAM misidentified by Zetsche, et al. Additionally, crystallographic data suggest that Cpf1 recognition of the PAM is based upon a shape readout of the dsDNA target, and the narrow minor groove typically adopted by AT-rich DNA aids the binding of a Cpf1 to a target. The polynucleotides and Class 2 Type II CRISPR Cas systems described in the present application may be used, for example, with a Cpf1 protein (e.g., from Francisella novicida) directed to a site on a target nucleic acid proximal to a 5′-TTTN-3′ PAM.

As used herein, the term “casPN” (Cas-associated polynucleotide, lacking a spacer sequence) refers to one or more polynucleotides that associate with a Class 2 CRISPR-Cas to form a nucleoprotein particle, wherein when the nucleoprotein particle is associated with a distinct spacer, the nucleoprotein particle is capable of site-directed binding to a target nucleic acid complementary to the target nucleic acid binding sequence of the spacer. Examples of Class 2 Type II CRISPR-Cas casPNs are illustrated in FIG. 1E, 110; FIG. 1F, 110; FIG. 2C, 210; and FIG. 2D, 210. Examples of Class 2 Type V CRISPR-Cas casPNs are illustrated in FIG. 3C, 306 and FIG. 3D, 306. In preferred embodiments of the present invention, a casPN is a single polynucleotide (e.g., FIG. 2C, 210; FIG. 3C, 306).

To facilitate understanding of the casPNs of the present invention, while not being bound by any theory, casPNs of the present invention can be described as follows. A casPN is capable of associating with a Class 2 CRISPR-Cas protein to form a Cas protein/casPN nucleoprotein complex, wherein the associating forms a nucleic acid sequence binding channel in the Cas protein/casPN complex capable of binding a nucleic acid sequence. However, a Cas protein/casPN nucleoprotein complex alone does not provide site-specific binding to a target nucleic acid sequence.

In some embodiments of the present invention, a casPN refers to a single-strand polynucleotide comprising a tracr element and/or specific secondary structures. In one embodiment, a casPN comprises a tracr element. When the casPN comprising the tracr element complexes with a Cas protein, the Cas protein more preferentially binds DNA sequences containing PAM sequences associated with the Cas protein than DNA sequences without PAM sequences.

Experiments performed in support of the present invention support that a Class 2 Type II CRISPR-Cas9 protein complexed with a sgRNA modified by removal of its spacer (forming a Cas9/sgRNA, modified by removal of its spacer, ribonucleoprotein complex) retains a higher binding affinity for DNA sequences containing PAM sequences associated with the ribonucleoprotein complex versus DNA sequences without such PAM sequences. In other words, the binding site distribution of the Class 2 Type II CRISPR-Cas9 protein complexed with a sgRNA modified by removal of its spacer is positively correlated with the PAM distribution in the DNA sequences.

For Class 2 Type II CRISPR-Cas systems of the present invention, a single-strand polynucleotide comprising a “tracr element,” as used herein, is lacking a spacer element. When the first polynucleotide is complexed with a cognate Cas protein it results in binding of the tracr element to the Cas protein providing a tracr element/Cas protein complex that more preferentially binds DNA sequences containing PAM sequences associated with the tracr element/Cas protein complex compared to DNA sequences without PAM sequences. In another embodiment, a single-strand polynucleotide comprising a tracr element comprises particular secondary structure, the secondary structure comprising a first stem element and a nexus element wherein the nexus element is located 3′ of the first stem element (as discussed herein there is the proviso that this first polynucleotide does not comprise a DNA target binding sequence). Thus, in one embodiment a casPN for Class 2 Type II CRISPR Cas systems can be characterized as follows. When a single-strand casPN comprising a tracer element is complexed with a cognate Cas9 protein it results in binding of the Cas9 protein to the single-strand polynucleotide to form a complex comprising a Cas9/tracr element complex that more preferentially binds DNA sequences containing the Cas9 related PAM sequences compared to DNA sequences without such PAM sequences. As described herein, the casPN (e.g., FIG. 2C, 210; FIG. 2D, 210) does not comprise a spacer element (e.g., FIG. 2C, 201; FIG. 2D, 201).

In some embodiments for Class 2 Type II CRISPR Cas systems, a casPN comprises specific secondary structures. For example, the casPN can be a first polynucleotide, having a 5′ end and a 3′ end, the first polynucleotide comprising a first stem element and a nexus element wherein the nexus element is located 3′ of the first stem element (as defined herein a casPN does not comprise a target nucleic acid binding sequence (i.e., there is the proviso that a casPN does not comprise a target nucleic acid binding sequence, e.g., a target DNA binding sequence)). In one embodiment, the first stem element of the casPN comprises, in a 5′ to 3′ direction, a lower stem sequence 1, a bulge sequence 1, an upper stem sequence 1, a loop sequence, an upper stem sequence 2 (wherein the upper stem sequence 1 and the upper stem sequence 2 form an upper stem element by base-pair hydrogen bonding between the upper stem sequence 1 and the upper stem sequence 2), a bulge sequence 2, a lower stem sequence 2 (wherein the lower stem sequence 1 and lower stem sequence 2 form the first stem element by base-pair hydrogen bonding between the first lower stem sequence and second lower stem sequence. In another embodiment, the casPN comprises in a 5′ to 3′ direction a stem sequence 1, a loop sequence, and a stem sequence2, wherein the stem sequence 1 and the stem sequence 2 form a first stem element by base-pair hydrogen bonding between the stem sequence 1 and the stem sequence 2.

In other aspects of the present invention, as described herein, a Class 2 Type II CRISPR-Cas casPN comprises more than one polynucleotide that forms a tracr element (e.g., FIG. 1E, 110; FIG. 1F, 110) and does not comprise a spacer element (e.g., FIG. 1E, 101; FIG. 1F, 101).

In some embodiments of the invention for Class 2 Type V CRISPR-Cas systems, a casPN comprises specific secondary structure that associates with a Class 2 Type V CRISPR-Cas protein (a casPN as defined herein does not contain a target nucleic acid binding sequence (i.e., there is the proviso that the casPN does not contain a spacer element)). An example of such a specific secondary structure is a single-strand polynucleotide comprising the specific secondary structure referred to herein as a “pseudoknot element” (e.g., FIG. 3C, 306).

In embodiment, casPN is capable of associating with a Class 2 Type V CRISPR-Cas protein to form a casPN/Cpf1 nucleoprotein complex, and the associating forms a nucleic acid sequence binding channel in the casPN/Cpf1 nucleoprotein complex capable of binding a nucleic acid sequence.

In other aspects of the present invention, the casPN comprises more than one polynucleotide that forms a pseudoknot element (e.g., FIG. 3D, 306) and does not comprise a spacer element (e.g., FIG. 3D, 302).

In view of the teachings of the present specification, one of ordinary skill in the art can select a Cas protein and the Cas polynucleotides associated therewith (e.g., Cas9 associated tracrRNA/crRNA or Cpf-1 associated crRNA) and engineer a casPN that can form a complex with the Cas protein. The site-specific binding of and/or cutting by of a nucleoprotein complex comprising the casPN, as well as modifications thereof (e.g., introduction of an affinity tag) can be confirmed, if necessary, using the Cas cleavage assay described in Example 3, an electrophoretic mobility shift assay (Garner, M., and Revzin, A., “A gel electrophoresis method for quantifying the binding of proteins to specific DNA regions: application to components of the Escherichia coli lactose operon regulatory system,” Nucleic Acids Res. 9 (13): 3047-60 (1981); Fried, M., Crothers, D., “Equilibria and kinetics of lac repressor-operator interactions by polyacrylamide gel electrophoresis,” Nucleic Acids Res. 9 (23): 6505-25 (1981); Fried, M., “Measurement of protein-DNA interaction parameters by electrophoresis mobility shift assay,” Electrophoresis 10, 366-376 (1989); Gagnon, K., and Maxwell, E., “Electrophoretic mobility shift assay for characterizing RNA-protein interaction,” Methods Mol Biol. 703:275-91 (2011); Fillebeen, C., et al., “Electrophoretic mobility shift assay (EMSA) for the study of RNA-protein interactions: the IRE/IRP example,” J Vis Exp. December 3(94) (2014)), to examine site-specific binding, and/or deep sequencing analysis to evaluate and compare the in cell activity (Example 4).

In some embodiments of the invention, the polynucleotide of the casPN is RNA (casRNA). For example, one embodiment of a casRNA is a casRNA that contains the structural elements of a corresponding Class 2 Type II CRISPR-cas sgRNA (the sgRNA being a component of a cognate sgRNA/Cas9 protein complex) with the exception that the spacer of the sgRNA is not present in the casRNA (see, e.g., an example of a casRNA as illustrated by FIG. 2C, 210). As another example, a casRNA is a casRNA that contains the structural elements of a corresponding Class II Type V CRISPR-Cas crRNA (the crRNA being a component of a cognate crRNA/Cpf1 protein complex) with the exception that the spacer of the crRNA is not present in the casRNA (see, e.g., an example of a casRNA as illustrated by FIG. 3C, 306). In other embodiments of the invention, the polynucleotide of the casPN is DNA (casDNA). In further embodiments of the invention, the polynucleotide of the casPN comprises at least one nucleotide of RNA and at least one nucleotide of DNA (casRNA-DNA). Accordingly, casRNA, casDNA, and casRNA-DNA represent embodiments of the casPN of the present invention. In additional embodiments, the casPN comprises nucleic acids comprising modified backbone residues or linkages, including, but not limited to, phosphorothioates, phosphoramidates, methyl phosphonates, chiral-methyl phosphonates, 2-O-methyl ribonucleotides, peptide-nucleic acids, threose nucleic acids, locked nucleic acids, glycol nucleic acid, bridged nucleic acids and morpholino structures.

Example 1 describes the use of in vitro transcription to produce a casRNA. In the example, overlapping primers were used to generate DNA templates for a number of Cas RNA components, including casRNA-1 (SEQ ID NO. 19). In vitro transcription of the DNA templates was carried out using a T7 promoter and a T7 RNA polymerase.

Sternberg, S. H., et al., (“DNA interrogation by the CRISPR RNA-guided endonuclease Cas9,” Nature. 2014 Mar. 6; 507(7490): 62-67)) teach methods using double-tethered DNA curtains to examine the locations and corresponding lifetimes of all binding events for tracrRNA/crRNA/Cas with DNA. Following the guidance of the present specification, one of ordinary skill in the art can apply such methods to evaluate preferential binding (higher binding affinity) of, for example, casRNA/Cas protein complexes of the present invention to DNA sequences containing PAM sequences versus DNA sequences without PAM sequences to confirm presence of a tracr element in the casRNA.

With reference to a crRNA or sgRNA, a “spacer” or “spacer element” as used herein refers to the polynucleotide sequence that can specifically hybridize to a target nucleic acid sequence (e.g., to direct site-specific binding of a crRNA/Cpf1 ribonucleoprotein complex, a sgRNA/Cas9 ribonucleoprotein complex, or a tracrRNA/crRNA ribonucleoprotein complex to the target nucleic acid sequence). In some embodiments the spacer element is a 100% complementary to the target nucleic acid sequence. In some embodiments the spacer element is less than 100% complementary to the target nucleic acid sequence but still capable of directing site-specific binding of a crRNA/Cpf1 ribonucleoprotein complex, a sgRNA/Cas9 ribonucleoprotein complex, or a tracrRNA/crRNA ribonucleoprotein complex to the target nucleic acid sequence. The spacer element interacts with the target nucleic acid sequence through hydrogen bonding between complementary base pairs (i.e., paired bases). A spacer element binds, for example, to a selected target DNA sequence and thus is a target DNA binding sequence.

The spacer element determines the location of site-specific binding and endonucleolytic cleavage for an associated Cas protein. Spacer elements range from ˜17- to ˜84 nucleotides long, depending on the Cas protein with which they are associated, and have an average length of 36 nucleotides (Marraffini, L. A., et al., “CRISPR interference: RNA-directed adaptive immunity in bacteria and archaea,” Nature Reviews Genetics. 2010; 11(3):181-190). For example, for SpyCas9 complexes the functional length for a spacer element to direct specific cleavage is typically about 12-25 nucleotides. Variability of the functional length for a spacer element is known in the art (e.g., U.S. Published Patent Application No. 2014-0315985, published 23 Oct. 2014). As another example, for Acidaminococcus spp. Cpf1 complexes the functional length for a spacer element to direct specific cleavage is typically about 16-25 nucleotides.

The term “spacer element sequence polynucleotide (sesPN)” as used herein refers to a single-strand polynucleotide comprising a spacer element (i.e., a polynucleotide sequence for binding to a selected target nucleic acid sequence (e.g., DNA); that is, an sesPN comprises a target nucleic acid binding sequence), with the provisos that, in a selected Class 2 CRISPR-Cas system, (i) a sesPN is a distinct polynucleotide relative to the casPN (e.g., FIG. 2C, 201; FIG. 2D, 201; FIG. 3C, 302), and (ii) the sesPN does not form base-pair hydrogen bonds with the casPN. In one embodiment, the sesPN does not form base-pair hydrogen bonds with the casPN that form a stable secondary structure. In another embodiment, the sesPN does not interact with the casPN in the absence of a cognate Cas protein.

In one embodiment of the invention, the polynucleotide of the sesPN is DNA (sesDNA). In another embodiment of the invention, the polynucleotide of the sesPN is RNA (sesRNA). In yet another embodiment of the invention, the polynucleotide of the sesPN comprises at least one nucleotide of RNA and at least one nucleotide of DNA (sesRNA-DNA). Accordingly, sesDNA, sesRNA, and sesRNA-DNA represent embodiments of sesPNs of the present invention. In additional embodiments, the sesPN comprises nucleic acids comprising modified backbone residues or linkages, including, but not limited to, phosphorothioates, phosphoramidates, methyl phosphonates, chiral-methyl phosphonates, 2-O-methyl ribonucleotides, peptide-nucleic acids, threose nucleic acids, locked nucleic acids, glycol nucleic acid, bridged nucleic acids, and morpholino structures.

sesPNs are typically synthesized based on sequences provided to commercial manufacturers. Other methods to make the sesPNs include polymerase chain reaction for sesDNAs, reverse transcription from RNA templates for sesDNAs, and in vitro transcription from DNA templates for sesRNAs.

In one embodiment, to determine whether a sesPN forms base-pair hydrogen bonds with a casPN the secondary structure of each polynucleotide is predicted (see, e.g., Ran, F. A., et al., “In vivo genome editing using Staphylococcus aureus Cas9,” Nature, 520(7546):186-91 (2015); Zuker, M. Mfold web server for nucleic acid folding and hybridization prediction, Nucleic Acids Res. 31, 3406-3415 (2003)). For Class 2 Type II CRISPR Cas systems, unpaired bases at the 3′ end of the sesPN are compared to unpaired bases at the 5′ end of the casPN to evaluate the possibility of the unpaired bases forming hydrogen bonds between the polynucleotides. For Class 2 Type V CRISPR Cas systems, unpaired bases at the 5′ end of the sesPN are compared to unpaired bases at the 3′ end of the casPN to evaluate the possibility of the unpaired bases forming hydrogen bonds between the polynucleotides.

In addition, the creation of stable secondary structure between two polynucleotides through base-pair hydrogen bonding can be determined by a number of methods known to those of ordinary skill in the art (e.g., experimental techniques, including but not limited to X-ray crystallography, Nuclear Magnetic Resonance (NMR) spectroscopy, Cryo-electron microscopy (Cryo-EM), Chemical/enzymatic probing, thermal denaturation (melting studies), and Mass spectrometry; predictive techniques, such as computational structure prediction; preferred methods include Chemical/enzymatic probing, thermal denaturation (melting studies)). Methods to predict secondary structures of single-strand RNA or DNA sequences are known in the art, for example, the “RNAfold web server” (rna.tbi.univie.ac.at/cgi-bin/RNAfold.cgi) predicts secondary structures of single-strand RNA or DNA sequences (see, e.g., Gruber A R, et al., The Vienna RNA Websuite, Nucleic Acids Res. 2008; Lorenz, R., et al., (2011) “ViennaRNA Package 2.0”, Algorithms for Molecular Biology, 6, 26). A preferred method to evaluate RNA secondary structure is to use the combined experimental and computational SHAPE method (Low J. T., et al., “SHAPE-Directed RNA Secondary Structure Prediction,” Methods (San Diego, Calif.) 2010; 52(2):150-158).

One empirical method to determine whether there is stable secondary structure (created by base-pair hydrogen bonding) formed between a casPN and a sesPN is analysis on non-denaturing gels (see, e.g., McGookin, R., “Gel electrophoresis of RNA in agarose and polyacrylamide under non-denaturing conditions,” Methods Mol Biol. 1985; 2:93-100). In this method, casPN and sesPN are combined in equal molar concentrations in an annealing or hybridization buffer (e.g., 1.25 mM HEPES, 0.625 mM MgCl₂, 9.375 mM KCl at pH7.5; or 20 mM Tris-HCl pH 7.5, 100 mM KCl, 5 mM MgCl₂), incubated above the melting temperature of the casPN and sesPN, and allowed to equilibrate at room temperature. This reannealed mixture of polynucleotides is a “combined” casPN/sesPN. The same equal molar concentrations of casPN and sesPN are separately denatured, separately reannealed, and then combined (“separate” casPN/sesPN). The combined and separate samples are resolved side by side on non-denaturing gels. The banding patterns of the combined and separate samples are compared. Formation of secondary structure is indicated by differences in the banding patterns between the combined and separate samples.

In some embodiments, a casPN is capable of interacting with a cognate Cas protein and a sesPN to form a casPN/sesPN/Cas nucleoprotein complex, wherein the binding of casPN to the Cas protein activates the complex for sesPN-guided DNA target binding. In preferred embodiments, the Class 2 CRISPR-Cas protein is a Cas9 protein or a Cpf1 protein.

In one embodiment, a Class 2 CRISPR-Cas nucleoprotein complex, comprises a Class 2 CRISPR-Cas protein and a Class 2 CRISPR-Cas associated polynucleotide lacking a spacer element (casPN); and a distinct spacer element sequence polynucleotide (sesPN) comprising a target nucleic acid binding sequence; wherein the Class 2 CRISPR-Cas nucleoprotein complex is capable of site-directed binding to a target nucleic acid complementary to the target nucleic acid binding sequence of the sesPN. In preferred embodiments, the Class 2 CRISPR-Cas protein is a Cas9 protein or a Cpf1 protein.

Another embodiment of the present invention is a composition comprising a casPN; wherein the casPN is capable of associating with (i) a Class 2 CRISPR-Cas protein and (ii) a distinct sesPN comprising a target nucleic acid binding sequence, thereby forming a Class 2 CRISPR-Cas nucleoprotein complex, and the Class 2 CRISPR-Cas nucleoprotein complex is capable of site-directed binding to a target nucleic acid complementary to the target nucleic acid binding sequence of the sesPN. In preferred embodiments, the Class 2 CRISPR-Cas protein is a Cas9 protein or a Cpf1 protein.

Example 3 describes the use of in vitro Cas cleavage assays to evaluate and compare the percent cleavage of selected Cas protein/polynucleotide complexes relative to selected double-stranded target sequences. A double-stranded target DNA comprising AAVS-1 was produced as described in Example 2. In Example 3, the cleavage of the double-stranded target DNA (AAVS-1) was determined for the following polynucleotides complexed with a Cas9 protein: a sgRNA-AAVS1 (exemplary structure illustrated in FIG. 2A, wherein 201 corresponds to the spacer element), tracrRNA/crRNA-AAVS1 (exemplary structure illustrated in FIG. 1B, wherein 103 corresponds to the spacer element), casRNA-1/sesRNA-AAVS1 (exemplary structure illustrated in FIG. 2C, wherein 201 corresponds to the sesRNA comprising the spacer element, and 210 corresponds to the casRNA), and casRNA-1/sesDNA-AAVS1 (exemplary structure illustrated in FIG. 2C, wherein 201 corresponds to the sesDNA comprising the spacer element and 210 corresponds to the casRNA). The data obtained from these cleavage assays support that the Cas protein/casPN/sesPN nucleoprotein complexes as described herein facilitate Cas protein mediated site-specific cleavage of target double-stranded DNA.

Example 4 presents a method using deep sequencing analysis to evaluate and compare the in cell cleavage activity of Cas protein/casPN/sesPN nucleoprotein complexes of the present invention versus control complexes Cas protein/sgRNA and tracrRNA/crRNA.

Example 5 illustrates the use of sesPNs (e.g., sesRNAs and sesDNAs) to evaluate and compare the modification ability of a collection of sesPNs against a selected target genomic DNA region, for example, a human target genomic DNA sequence in cells.

Example 6 presents a method through which CRISPR RNAs (crRNAs) and trans-activating CRISPR RNAs (tracrRNAs) of Class 2 CRISPR-Cas systems can be identified. In addition, the example describes elements of designing casPNs and sesPNs.

Example 5 and Example 6 are described with reference to Class 2 Type II CRISPR-Cas systems but the methods are readily modifiable by one of ordinary skill in the art to be applied to other Class 2 CRISPR-Cas systems, for example, Class 2 Type V CRISPR-Cas systems.

The term “affinity tag” as used herein refers to one or more moiety that increases the binding affinity of a sesPN to a casPN/Cas protein complex, a casPN to a Cas protein, or a sesPN to a Cas protein. Affinity tags can be introduced into one or more of the following components of a Class 2 CRISPR-Cas system of the present invention: a Cas protein, a sesPN, a casPN, or combinations thereof. Some embodiments of the present invention use an “affinity sequence,” which is a polynucleotide sequence comprising one or more affinity tag. In some embodiments of the present invention, the sesPN comprises an affinity sequence wherein the affinity sequence is located 5′ to the target nucleic acid binding sequence, 3′ to the target nucleic acid binding sequence, or both 5′ and 3′ to the target nucleic acid binding sequence in the sesPN. Some embodiments of the present invention introduce one or more affinity tag to the N-terminal of a Cas protein sequence, to the C-terminal of a Cas protein sequence, to a position located between the N-terminal and C-terminal of a Cas protein sequence, and combinations thereof. In some embodiments of the invention the Cas-polypeptide is modified with an affinity tag or an affinity sequence. In some embodiments of the present invention, the casPN comprises an affinity sequence wherein the affinity sequence is located at the 5′ end, at the 3′ end, at both the 5′ and 3′ ends, at a position between the 5′ and 3′ ends, and combinations thereof.

In some embodiments of the invention affinity tags are introduced into the sesPN and the Cas protein of a cognate casPN/Cas protein complex, the casPN and the Cas protein of a cognate casPN/Cas protein complex, or the sesPN, the casPN, and the Cas protein of a cognate casPN/Cas protein complex. For example, an affinity sequence of the sesPN can be modified using a MS2 binding sequence, U1A binding sequence, stem-loop sequence (e.g., a Csy4 protein binding sequence, or Cas6 protein binding sequence), eIF4A binding sequence, Transcription activator-like effector (TALE) binding sequence (Valton, J., et al., “Overcoming Transcription Activator-like Effector (TALE) DNA Binding Domain Sensitivity to Cytosine Methylation” J Biol Chem. 2012 Nov. 9; 287(46): 38427-38432), or zinc finger domain binding sequence (Font, J., et al., “Beyond DNA: zinc finger domains as RNA-binding modules,” Methods Mol Biol. 2010; 649:479-91; Isalan, M., et al., “A rapid, generally applicable method to engineer zinc fingers illustrated by targeting the HIV-1 promoter,” Nat Biotechnol. 2001 July; 19(7): 656-660). In some embodiments, the casPN can be similarly modified, or both the sesPN and the casPN can be modified. The Cas protein coding sequence is then modified to comprise a corresponding affinity tag: an MS2 coding sequence, U1A coding sequence, stem-loop binding protein coding sequence (e.g., an enzymatically inactive Csy4 protein that binds the Csy4 protein sequence), eIF4A coding sequence, TALE coding sequence, or a zinc finger domain coding sequence, respectively. When both the casPN and the sesPN are modified with an affinity sequence, in preferred embodiments, the two affinity sequences typically are not the same; thus, there are two different binding sequences associated with the Cas protein. In one embodiment, the affinity sequence is a nucleic acid binding protein binding sequence (e.g., the binding sequence corresponding to a DNA binding protein or the binding sequence corresponding to an RNA binding protein) or nucleic acid binding domain thereof and the affinity tag is the corresponding nucleic acid binding protein (e.g., MS2 protein and its corresponding RNA binding sequence; U1A protein and its corresponding RNA binding sequence; a transcription factor protein and its corresponding DNA binding sequence; a zinc finger and its corresponding DNA or RNA binding sequence; a Csy4 protein and its corresponding RNA binding sequence). Typically, enzymatically inactive nucleic acid binding proteins that retain sequence specific nucleic acid binding are used; however, in some embodiments enzymatically active nucleic acid binding proteins or nucleic acid proteins with altered enzymatic activity are used.

In some embodiments, the sesPN is tethered to the Cas protein at a location to bring the sesPN into proximity with the RNA/DNA binding channel of the Cas protein. In some embodiments, the casPN is tethered to the Cas protein at a location to stabilize the casPN/Cas protein interaction.

Example 8 and Example 11A, respectively, describe the use of a Cas9 fusion with the RNA binding protein dCsy4 (an enzymatically inactive variant of the Pseudomonas aeruginosa (strain UCBPP-PA14)) and a sesPN modified to include the corresponding Csy4 RNA binding sequence (i.e., an affinity sequence) at the 5′ end of the sesPN, and use of a Cpf1 fusion with an RNA binding protein dCsy4 and a sesPN modified to include the corresponding Csy4 RNA binding sequence (i.e., an affinity sequence) at the 5′ end of the sesPN. The combination of these Cas proteins/dCsy4 binding domain fusion proteins and attachment of the corresponding RNA binding protein binding sequence to an sesPN illustrates a mechanism that can be used to bring the sesPN into proximity with the RNA/DNA binding channel of the Cas protein.

Example 11B provides an example of tethering both a sesPN and a casPN to a fusion protein comprising a cognate Cas protein and two dCsy4 RNA binding domains that each bind a different RNA binding sequences (i.e., two different affinity sequences). Using the two different affinity sequences and their corresponding RNA binding domains ensures that the sesPN and casPN are tethered to the appropriate locations of the Cas protein component of the fusion protein. The sesPN is tethered at a location to bring the sesPN into proximity with the RNA/DNA binding channel of the Cas protein. The casPN is tethered at a location to stabilize the casPN/Cas protein interaction.

A wide variety of affinity tags are disclosed in U.S. Published Patent Application No. 2014-0315985 (published 23 Oct. 2014).

The term “cross-linking moiety” as used herein refers to a moiety suitable to provide cross-linking between a sesPN and the Cas protein of a cognate casPN/Cas protein complex, the casPN and the Cas protein of a cognate casPN/Cas protein complex, or the sesPN, the casPN, and the Cas protein of a cognate casPN/Cas protein complex. A cross-linking moiety is another example of an affinity tag.

Examples of cross-linking targets include, but are not limited to, amines (eg, lysines, protein or peptide N-terminus), sulfhydryls (cysteines), carbohydrates (oxidized sugars), and carboxyls (protein or peptide C-terminus, aspartic acid, glutamic acid).

Examples of chemical cross-linking groups include, but are not limited to, carbodiimide, N-hydroxysuccinimide esters (NHS) ester, imidoesters, maleimides, haloacetyls, pyridyldisulfides, hydrazides, alkoxyamines, diazirines, aryl azides, and isocyanates.

A wide variety of nucleic acid/protein cross-linking moieties are commercially available to one of ordinary skill in the art, including, but not limited to thiols (e.g., 5′ thiol C6, dithiol phosphoramidite (DTPA), and 3′ thiol C3) (e.g., Integrated DNA Technologies, Inc., Coralville, Iowa; Thermo Fisher Scientific, South San Francisco, Calif.; ProteoChem, Loves Park, Ill.; BroadPharm, San Diego, Calif.).

Following the guidance of the present specification, one of ordinary skill in the art can modify the sesPNs, casPNs, and Cas proteins with cross-linking moieties using established chemical methods (e.g., Methods of Chemistry of Protein and Nucleic Acid Cross-Linking and Conjugation, Second Edition, by Shan S. Wong and David M. Jameson, Oct. 10, 2011, published by CRC Press, ISBN-13 978-0849374913; Bioconjugate Techniques, Third Edition, by Greg T. Hermanson, Sep. 2, 2013, published by Academic Press, ISBN-13 978-0123822390; Chemistry of Bioconjugates: Synthesis, Characterization, and Biomedical Applications, First Edition, by Ravin Narain (Editor), Feb. 3, 2014, published by Wiley, ISBN-13 978-1118359143; Bioconjugation Protocols: Strategies and Methods (Methods in Molecular Biology), Second Edition, by Sonny S. Mark (Editor), Series: Methods in Molecular Biology (Book 751), Jun. 23, 2011, published by Humana Press, ISBN-13 978-1617791505; Crosslinking Technical Handbook, Thermo Fisher Scientific, South San Francisco, Calif.). In some embodiments, the Cas protein primary sequence is engineered to comprise an amino acid residue (e.g., a Cys amino acid residue) useful for cross-linking to a cross-linking moiety present in the sesPN or casPN at a particular residue position in the Cas protein (e.g., substitution or insertion of a Cys amino acid at a position that is not a Cys amino acid in the cognate wild-type Cas protein). Example 7, Example 9, and Example 10 provide examples of modifications of a Cas protein primary sequence.

Another example of a cross-linking moiety is to provide one or more photoactive nucleotide in a polynucleotide sequence of the sesPN and/or casPN that is positioned to maximize contact between the one or more photoactive nucleotide and one or more photoreactive amino acid and use UV light to induce cross-linking between the one or more photoactive nucleotide and the one or more photoreactive amino acid. In one embodiment, a cross-linking moiety for use in the practice of the present invention is a cross-linkable polynucleotide comprising a contiguous run of uracil nucleotides (poly-U) or a run of uracil nucleotides alternating with other nucleotides. In another embodiment, a cross-linking moiety for use in the practice of the present invention is a cross-linkable polynucleotide comprising a contiguous run of thymidine nucleotides (poly-T) or a run of thymidine nucleotides alternating with other nucleotides. Such cross-linkable polynucleotides are, for example, positioned in the sesPN and/or casPN to maximize contact with one or more photoreactive amino acids of a Cas protein. A large number of photoreactive amino acids can be added photochemically (e.g., 254 nm) to uracil (Smith, K. C., and Shetlar, M. D., “DNA-Protein Crosslinks,” available at www.photobiology.info/Smith_Shetlar.html) including glycine, serine, phenylalanine, tyrosine, tryptophan, cystine, cysteine, methionine, histidine, arginine and lysine. The most reactive amino acids are phenylalanine, tyrosine and cysteine. A number of photoreactive amino acids can be added photochemically to thymidine (Smith, K. C., and Shetlar, M. D., “DNA-Protein Crosslinks,” available at www.photobiology.info/Smith_Shetlar.html) including lysine, arginine, cysteine and cystine. Accordingly, regions of a casPN/Cas protein complex comprising one or more photoreactive amino acid can be evaluated for their ability to act as cross-linking epitopes. Also, the Cas protein coding sequence can be modified to introduce a photoreactive amino acid (an affinity tag) in a position suitable to come into proximity of a photoactive nucleotide (an affinity tag) in an affinity sequence of a sesPN and/or a casPN.

Further examples of photoreactive cross-linking moieties include, but are not limited to, photo reactive amino acid analogs (L-photo leucine, L-photo-methionine, p-benzoyl-L-phenylalanine), and photoactivatable ribonucleosides (halogenated and thione containing ribonucleoside analogues, such as 5-Bromo-dUTP, Azide-PEG4-aminoallyl-dUTP, 4-thiouridine, 6-thioguanosine, preferred reaction with tyrosines, phenylalanines and tryptophanes). General photoreactive cross-linking moieties include, aryl azides, azido-methyl-coumarins, benzophenones, anthraquinones, certain diazo compounds, diazirines, and psoralen derivatives.

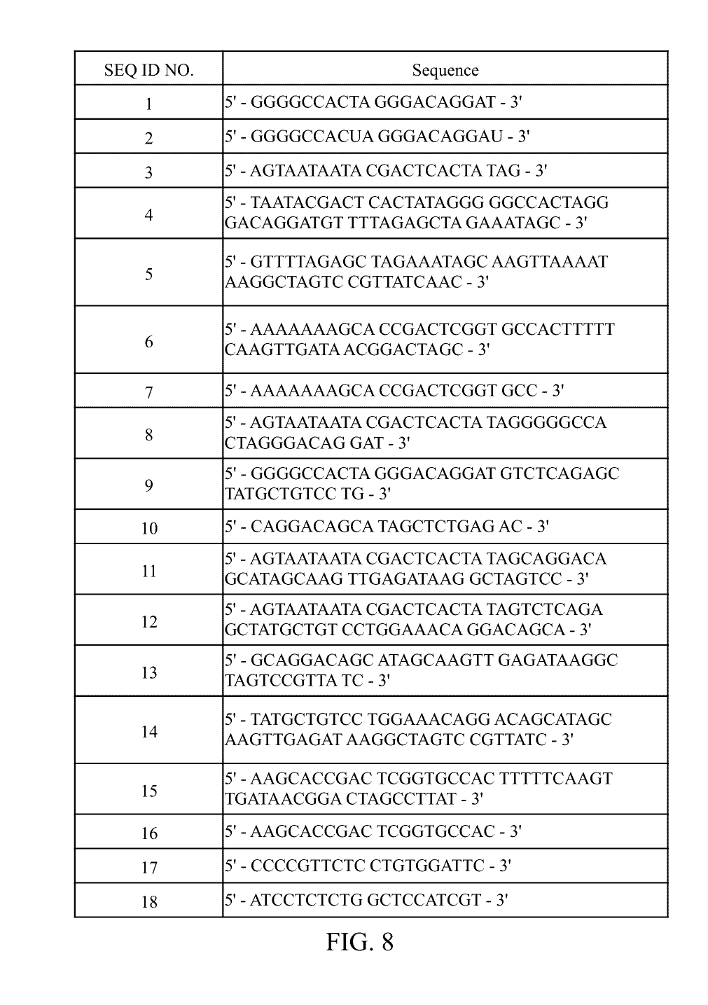

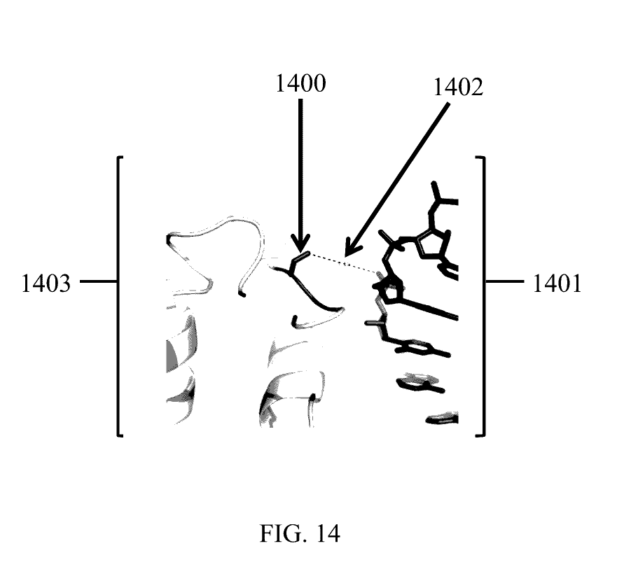

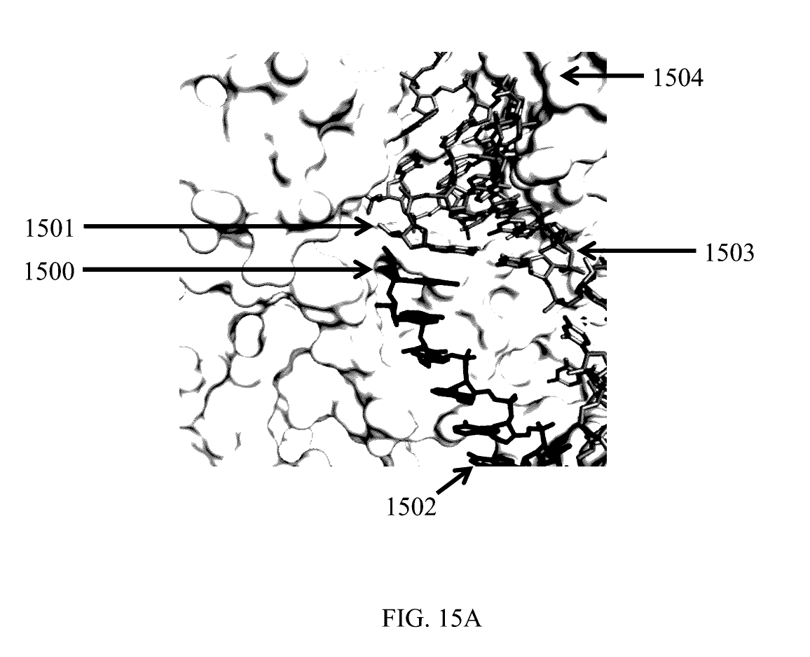

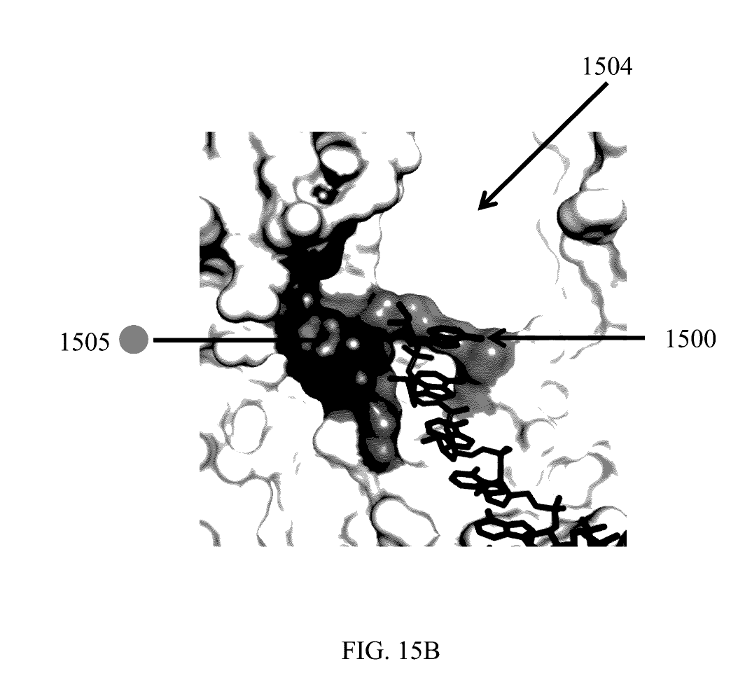

One example of a photoreactive amino acid of a wild-type Cas9 protein complexed with a sgRNA is represented in FIG. 12A (WTSpyCas9 Cys). Examples of sites for cross-linking epitopes of SpyCas9 located along the length of the spacer RNA of a sgRNA are illustrated in FIG. 8A and FIG. 8B. FIG. 14 presents an example of a serine in the helical domain of SpyCas9 in close proximity to a sesPN. FIG. 15A shows the relationship of the 3′ end of the sesPN to the 5′ end of the casPN. FIG. 15B shows a representation of the 3′ end of a sesPN in proximity to cross-linking epitopes of the helical domain of SpyCas9.

There are a number of photocross-linking analogs that serve as substrates for RNA polymerases for introduction into RNA molecules including 4-thio-UTP, 5-azido-UTP, 5-bromo-UTP and 8-azido-ATP, 5-APAS-UTP, 5-APAS-CTP, 8-APAS-ATP, and 8-N(3)AMP (C. Costas, et al., “RNA-protein cross-linking to AMP residues at internal positions in RNA with a new photocross-linking ATP analog,” Nucleic Acids Res., 2000, 28(9): 1849-1858; Gaur R. K., “T7 RNA polymerase-mediated incorporation of 8-N(3)AMP into RNA for studying protein-RNA interactions,” Methods Mol Biol. 2008; 488:167-80).

A variety of cross-linking methods and moieties are commercially available, for example, from TriLink Biotechnologies (San Diego, Calif.) including, for photocross-linking: RNA-4-Thiouridine, 5-Bromouridine-5′-Triphosphate, 5-Iodouridine-5′-Triphosphate, 4-Thiouridine-5′-Triphosphate/DNA-6-Thio-dG, 4-Thiothymidine.

Examples of general cross-linking reagents include, but are not limited to, glutaraldehyde, formaldehyde. Furthermore, monofunctional (e.g., one functional cross-linking moieties, such as alkyl imidates) and bifunctional (two cross-linking moieties, disuccinimidyl suberate (DSS)) or trifunctional cross-linking moieties can be used, as well as homobifunctional (DSS) and heterobifunctional (sulfosuccinimidyl-4-(N-maleimidomethyl) cyclohexane-1-carboxylate (Sulfo-SMCC)) cross-linking moieties. Additionally, cross-linking moieties can comprise different spacer lengths (C3, C6, PEG spacers, and others).

In some embodiments, the sesPN is cross-linked to a residue of the Cas protein at a location to bring the sesPN into proximity with the RNA/DNA binding channel of the Cas protein. In some embodiments, the casPN is tethered to a residue of the Cas protein at a location to stabilize the casPN/Cas protein interaction.

Example 7 describes the modification of sesPNs of the present invention to include a cross-linking agent, as well as modification of selected amino acid residues in the Class 2 Type II CRISPR-Cas9 protein. The results of the Cas cleavage assays using the AAVS-1 target double-stranded DNA (Example 2) and the Cas9-Cys/thiolated sesRNA/casRNA-2 RNP complexes are summarized in Table 3. The biochemical cleavage data for the Cas9-Cys/thiolated sesRNA/casRNA-2 RNP complexes demonstrate that the Cas9-Cys/thiolated sesRNA/casRNA constructs as described herein facilitate Cas mediated site-specific cleavage of target double-stranded DNA.

Example 9 describes the modification of sesPNs of the present invention to include a cross-linking agent, as well as modification of selected amino acid residues in the CRISPR-Cas Class 2 Type V CRISPR Cpf1 protein. This combination of a modified Cas protein and modified sesPN provides another example of using cross-linking to bring the sesPN into proximity with the RNA/DNA binding channel of the Cas protein.

Example 10 describes a combination of a modified Cpf1 protein, modified sesPN, and modified Cpf1 casPN. In this example, the sesPN is modified using a thiol cross-linking moiety to tether it to the Cpf1 protein and the casPN is modified using a UV-cross-linkable moiety to tether it to the Cpf1 protein. The sesPN is tethered at a location to bring the sesPN into proximity with the RNA/DNA binding channel of the Cpf1 protein. The casPN is tethered at a location to stabilize the casPN/Cpf1 protein interaction.

The terms “ligand” and “ligand binding moiety” as used herein refer to moieties that facilitate the binding of a sesPN and to the Cas protein of a cognate casPN/Cas protein complex, the casPN and the Cas protein of a cognate casPN/Cas protein complex, or the sesPN, the casPN, and the Cas protein of a cognate casPN/Cas protein complex. Ligands and ligand binding moieties are paired affinity tags.

One embodiment of use of a ligand moiety is to build a ligand-binding moiety into the Cas protein and modify a polynucleotide sequence of the sesPN and/or casPN to contain the ligand. A ligand/ligand binding moiety useful in the practice of the present invention is avidin or streptavidin/Biotin (see, e.g., Livnah, O, et al., “Three-dimensional structures of avidin and the avidin-biotin complex,” Proceedings of the National Academy of Sciences of the United States of America, 1993; 90(11):5076-5080; Airenne, K. J., et al., “Recombinant avidin and avidin-fusion proteins,” Biomol Eng. 1999 Dec. 31; 16(1-4):87-92.). One example of a Cas protein with a ligand binding moiety is a Cas protein fused to a ligand avidin or streptavidin designed to bind a 5′ or 3′ biotinylated sesPN, wherein the sesPN comprises a polynucleotide sequence with which the biotin is associated in addition to the DNA target binding sequence of the sesPN (“sesPN-biotin”). Biotin is a high affinity and high specificity ligand for the avidin or streptavidin protein. By fusing an avidin or streptavidin polypeptide chain to the Cas protein, the Cas protein has a high affinity and specificity for a 5′ or 3′ biotinylated sesPN-biotin.

The sequence of a selected sesPN and the biotin can be determined. Biotinylation is preferably in close proximity to the 5′ or 3′ ends of the sesPN. The sequence of the sesPN and location of the biotin is provided to commercial manufacturers for synthesis of the sesPN-biotin or can be added through the use of an artificial third basepair (Ds-Pa) in an in-vitro translation reaction (Hirao, et al., “An unnatural hydrophobic base pair system: site-specific incorporation of nucleotide analogs into DNA and RNA,” Nature Methods 3(9):729-735 (2006)). casPNs can be similarly modified at the 5′ end, the 3′ end or positions between the 5′ end and the 3′ end. Changes to cleavage percentage and specificity of the ligand-binding modified Cas/ligand sesPN and/or casPN are evaluated as described below in Example 3 and Example 4.

Examples of other ligands and ligand binding moieties that can be similarly used include, but are not limited to (ligand/ligand binding moiety): estradiol/estrogen receptor (see, e.g., Zuo, J., et al., “Technical advance: An estrogen receptor-based transactivator XVE mediates highly inducible gene expression in transgenic plants,” Plant J. 2000 October; 24(2):265-73), rapamycin/FKBP12, and FK506/FKKBP (see, e.g., Setscrew, B., et al., “A split-Cas9 architecture for inducible genome editing and transcription modulation,” Nature Biotechnology 33, 139-142 (2015); Chiu M. I., et al., “RAPT1, a mammalian homolog of yeast Tor, interacts with the FKBP12/rapamycin complex,” PNAS 1994; 91(26):12574-12578).

Another example of a ligand and ligand-binding moiety is to provide one or more aptamer or modified aptamer in a polynucleotide sequence of a sesPN that has a high affinity and binding specificity for a selected region of a casPN/Cas protein complex or the Cas protein thereof. Furthermore, a casPN can comprise one or more aptamer or modified aptamer in its polynucleotide sequence that has a high affinity and binding specificity for a selected region the cognate Cas protein for the casPN. In one embodiment, a ligand binding moiety is a polynucleotide comprising an aptamer (see, e.g., Navani, N. K., et al., “In vitro Selection of Protein-Binding DNA Aptamers as Ligands for Biosensing Applications,” Biosensors and Biodetection, Methods in Molecular Biology™ Volume 504, 2009, pp 399-415; A. V. Kulbachinskiy, “Methods for Selection of Aptamers to Protein Targets,” Biochemistry (Moscow), Vol. 72, No. 13, pp. 1505-1518 (2007)). Aptamers are single-strand functional nucleic acids that possess recognition capability of a corresponding ligand. Typically, the aptamer is located at the 5′ or 3′ end of the sesPN or in casPNs at the 5′ end, the 3′ end, or a position between the 5′ and 3′ ends. In the practice of the present invention one example of a ligand is a casPN/Cas complex. Another example of a ligand is the Cas protein, portions thereof, or modified regions of a Cas fusion protein.

In another embodiment, a ligand binding moiety comprises a modified polynucleotide wherein a nonnative functional group is introduced at positions oriented away from the hydrogen bonding face of the bases of the modified polynucleotide, such as the 5-position of pyrimidines and the 8-position of purines (“Slow Off-rate Modified Aptamers or SOMAmers”; see, e.g., Rohloff, J. C., et al., “Nucleic Acid Ligands With Protein-like Side Chains: Modified Aptamers and Their Use as Diagnostic and Therapeutic Agents,” Molecular Therapy Nucleic Acids (2014) 3, e201). An aptamer with high specificity and affinity for Cas proteins could be obtained by in vitro selection and screening of an aptamer library.

In yet another embodiment, an established aptamer binding sequence/aptamer is used by introducing the aptamer-binding region into the Cas protein. For example, a biotin-binding aptamer can be introduced 5′ or 3′ of the DNA-binding region of a sesPN and the Cas protein can be selectively biotinylated to form a corresponding binding site for the biotin-binding aptamer.

The creation of a high affinity binding site for a selected ligand on a Cas protein can be achieved using several protein engineering methods known to those of ordinary skill in the art in view of the guidance of the present specification. Examples of such protein engineering methods include, rational protein design, directed evolution using different selection and screening methods for the library (e.g. phage display, ribosome display, yeast display, RNA display), DNA shuffling, computational methods (e.g. ROSETTA, www.rosettacommons.org/software), or introduction of a known high affinity ligand into Cas. Libraries obtained by these methods can be screened to select for Cas protein high affinity binders using, for example, a phage display assay, a cell survival assay, or a binding assay.

In some embodiments, two or more different types of affinity tags can be introduced into one or more of the following components of a Class 2 CRISPR-Cas system of the present invention: a Cas protein, a sesPN, a casPN, or combinations thereof. For example, a sesPN can be cross-linked to a Cas protein comprising a fusion to a RNA binding protein and a casPN can comprise the RNA binding protein binding site for the RNA binding protein. As another example, a sesPN can comprise a ligand, a Cas protein can comprise a ligand binding moiety that binds the sesPN ligand, and a casPN can be cross-linked to the Cas protein using a photoactive cross-linking moiety. Typically, if both a sesPN and a casPN are tethered to a Cas protein, the affinity tags for the sesPN and the casPN are different to maintain specificity of the site to which they are each tethered on the Cas protein.

One aspect of the invention relates to methods of manufacturing a casPN, a sesPN, or both a casPN and a sesPN of the present invention. In one embodiment, the method of manufacturing comprises chemically synthesizing a casPN, a sesPN, or both a casPN and a sesPN. In some embodiments, the casPN and/or sesPN comprise RNA bases, and can be generated from templates using in vitro transcription.

In one aspect, the present invention relates to expression cassettes comprising polynucleotide coding sequences for a sesDNA, a sesRNA, a casDNA, a casRNA, and/or a Cas protein. An expression cassette of the present invention at least comprises a polynucleotide encoding a casPN or sesPN of the present invention. Expression cassettes useful in the practice of the present invention can further include Cas protein coding sequences. In one embodiment, an expression cassette comprises a casPN coding sequence. In another embodiment, one or more expression cassette comprise a casPN coding sequence and a cognate Cas protein coding sequence. Expression cassettes typically comprise regulatory sequences that are involved in one or more of the following: regulation of transcription, post-transcriptional regulation, and regulation of translation. Expression cassettes can be introduced into a wide variety of organisms including bacterial cells, yeast cells, plant cells, and mammalian cells. Expression cassettes typically comprise functional regulatory sequences corresponding to the organism(s) into which they are being introduced.

One aspect of the present invention relates to vectors, including expression vectors, comprising polynucleotide coding sequences for a sesDNA, a sesRNA, a Cas DNA, a casRNA, and/or a Cas protein. Vectors useful for practicing the present invention include plasmids, viruses (including phage), and integratable DNA fragments (i.e., fragments integratable into the host genome by homologous recombination). A vector replicates and functions independently of the host genome, or may, in some instances, integrate into the genome itself. Suitable replicating vectors will contain a replicon and control sequences derived from species compatible with the intended expression host cell. Transformed host cells are cells that have been transformed or transfected with the vectors constructed using recombinant DNA techniques.

General methods for construction of expression vectors are known in the art. Expression vectors for most host cells are commercially available. There are several commercial software products designed to facilitate selection of appropriate vectors and construction thereof, such as insect cell vectors for insect cell transformation and gene expression in insect cells, plant cell vectors for plant cell transformation and gene expression in plant cells, bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transformation and gene expression and methods to easily enable cloning of such polynucleotides. SnapGene™ (GSL Biotech LLC, Chicago, Ill.; snapgene.com/resources/plasmid_files/your_time_is_valuable/), for example, provides an extensive list of vectors, individual vector sequences, and vector maps, as well as commercial sources for many of the vectors.

Expression vectors can also include polynucleotides encoding protein tags (e.g., poly-His tags, hemagglutinin tags, fluorescent protein tags, bioluminescent tags). The coding sequences for such protein tags can be fused to the Cas protein coding sequences or can be included in an expression cassette, for example, in a targeting vector.

In some embodiments, polynucleotides encoding sesDNA, sesRNA, casDNA, casRNA, and/or Cas protein are operably linked to an inducible promoter, a repressible promoter, or a constitutive promoter.

Methods of introducing polynucleotides (e.g., an expression vector) into host cells are known in the art and are typically selected based on the kind of host cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

In some embodiments of the present invention, it is useful to express all components of a sesPN/casPN/Cas protein system in a host cell. Expression of a sesRNA, a casRNA, and a Cas protein in a host cell can be accomplished through use of expression vectors with transcription promoters. However, expression of sesDNA or casDNA in a target cell is not accomplished with the use of standard cloning vectors. Single-strand DNA expression vectors, which can intracellularly generate single-strand DNA molecules, have been developed (Chen, Y., et al., “Intracellular production of DNA enzyme by a novel single-strand DNA expression vector,” Gene Ther. 2003 September; 10(20):1776-80; Miyata S., et al., “In vivo production of a stable single-strand cDNA in Saccharomyces cerevisiae by means of a bacterial retron,” Proc Natl Acad Sci USA 1992; 89: 5735-5739; Mirochnitchenko, O., et al., “Production of single-strand DNA in mammalian cells by means of a bacterial retron,” J Biol Chem 1994; 269: 2380-2383; Mao J., et al., “Gene regulation by antisense DNA produced in vivo. J Biol Chem 1995; 270: 19684-19687). Typically, these single-strand DNA expression vectors rely on transcription of a selected single-strand DNA sequence to form an RNA transcript that is the substrate for a reverse transcriptase and RNaseH to generate the selected single-strand DNA in a host cell. For example, components of single-strand DNA expression vectors often comprise, a reverse transcriptase coding sequence (e.g., a mouse Moloney leukemia viral reverse transcriptase gene), a reverse transcriptase primer binding site (PBS) as well as regions of the promoter that are essential for the reverse transcription initiation, the coding sequence of interest (e.g., a sesDNA or casDNA coding sequence), a stem loop structure designed for the termination of the reverse transcription reaction, and an RNA transcription promoter suitable for use in a host cell (used to create a mRNA template comprising the previous components). Reverse transcriptase expressed in cells uses endogenous tRNApro as a primer. After reverse transcription, single-strand DNA is released when the template mRNA is degraded either by endogenous RNase H or the RNase H activity of the reverse transcriptase (Chen, Y., et al., “Expression of ssDNA in Mammalian Cells,” BioTechniques 34:167-171 January 2003). Such expression vectors may be employed for expression of a sesDNA and casDNA of the present invention in a host cell.

Aspects of the present invention include, but are not limited to the following: one or more expression cassettes comprising polynucleotides encoding sesDNA, sesRNA, casDNA, casRNA, and/or Cas protein; one or more vectors, including expression vectors, comprising polynucleotides encoding sesDNA, sesRNA, casDNA, casRNA, and/or Cas protein; methods of manufacturing expression cassettes comprising polynucleotides encoding sesDNA, sesRNA, casDNA, casRNA, and/or Cas protein; methods of manufacturing vectors, including expression vectors, comprising polynucleotides encoding sesDNA, sesRNA, casDNA, casRNA, and/or Cas protein; methods of introducing one ore more expression cassettes, comprising polynucleotides encoding sesDNA, sesRNA, casDNA, casRNA, and/or Cas protein, into a selected host cell; methods of introducing one or more vectors, including expression vectors, comprising polynucleotides encoding sesDNA, sesRNA, casDNA, casRNA, and/or Cas protein, into a selected host cell; host cells comprising one or more expression cassettes (recombinant cells), comprising polynucleotides encoding sesDNA, sesRNA, casDNA, casRNA, and/or Cas protein; host cells comprising one or more vectors (recombinant cells), including expression vectors, comprising polynucleotides encoding sesDNA, sesRNA, casDNA, casRNA, and/or Cas protein; host cells comprising one or more polynucleotides encoding sesDNA, sesRNA, casDNA, casRNA, and/or Cas protein (recombinant cells); host cells (recombinant cells) expressing the products of one or more polynucleotides encoding sesDNA, sesRNA, casDNA, casRNA, and/or Cas protein; and methods for manufacturing sesDNA, sesRNA, casDNA, casRNA, and/or Cas protein, comprising isolating the sesDNA, sesRNA, casDNA, casRNA, and/or Cas protein from host cells (recombinant cells) expressing the products of one or more polynucleotides encoding sesDNA, sesRNA, casDNA, casRNA, and/or Cas protein.

Additional aspects of the present invention include, but are not limited to the following: a sesPN, a casPN, and/or a Cas protein modified as described herein; one or more nanoparticle comprising a sesPN, a casPN, a Cas protein (e.g., modified as described herein), a casPN/Cas protein nucleoprotein complex, and/or a Class 2 Type II nucleoprotein complex of the present invention (e.g., comprising a sesPN, a casPN, and a Cas protein); compositions comprising a sesPN, a casPN, and/or a Cas protein (e.g., modified as described herein), in some embodiments further comprising a buffer and/or container; kits comprising such compositions; methods of manufacturing a sesPN, a casPN, and/or a Cas protein (e.g., modified as described herein), for example, chemical synthesis; methods of introducing one or more Class 2 Type II nucleoprotein complexes of the present invention, sesPNs, casPNs, Cas proteins (e.g., modified as described herein), and/or casPN/Cas protein nucleoprotein complexes into a selected host cell, for example, by electroporation, lipofection, a gene gun or a biolistic particle delivery system; host cells comprising one or more Class 2 Type II nucleoprotein complexes of the present invention, sesPNs, casPNs, Cas proteins (e.g., modified as described herein), and/or casPN/Cas protein nucleoprotein complexes; and host cells comprising genomic DNA modified by a method using one or more Class 2 Type II nucleoprotein complexes of the present invention, sesPNs, casPNs, Cas proteins (e.g., modified as described herein), and/or casPN/Cas protein nucleoprotein complexes.

Another aspect of the present invention relates to methods to generate non-human genetically modified organisms. Generally, in these methods expression cassettes comprising polynucleotide sequences of the sesPN, casPN, and Cas protein, as well as a targeting vector are introduced into zygote cells to site-specifically introduce a selected polynucleotide sequence at a target DNA sequence in the genome to generate a modification of the genomic DNA. The selected polynucleotide sequence is present in the targeting vector and a complex of the sesPN/casPN/Cas protein contacts, binds, and cuts the target DNA sequence. Modifications of the genomic DNA typically include, insertion of a polynucleotide sequence, deletion of a polynucleotide sequence, or mutation of a polynucleotide sequence, for example, gene correction, gene replacement, gene tagging, transgene insertion, gene disruption, gene mutation, mutation of gene regulatory sequences, and so on. In one embodiment of methods to generate non-human genetically modified organisms, the organism is a mouse. In some embodiments of these methods, the Class 2 CRISPR-Cas nucleoprotein particles of the present invention or one or more component of the nucleoprotein particles (e.g., a sesPN, a casPN, and/or a Cas protein) are directly introduced into zygote cells. In some embodiments one or more other molecule, for example, an oligonucleotide and/or a donor polynucleotide are also directly introduced into zygote cells. One embodiment of this aspect of the invention is the generation of genetically modified mice.

Generating transgenic mice involves five basic steps (Cho A., et al., “Generation of Transgenic Mice,” Current protocols in cell biology, 2009; CHAPTER.Unit-19.11). First, purification of a transgenic construct (e.g., expression cassettes comprising polynucleotide sequences of the sesPN, casPN, and Cas protein, as well as a targeting vector, or complexes comprising the sesPN, the casPN, and the Cas protein). Second, harvesting donor zygotes. Third, microinjection of the transgenic construct into the mouse zygote. Fourth, implantation of microinjected zygotes into pseudo-pregnant recipient mice. Fifth, performing genotyping and analysis of the modification of the genomic DNA established in founder mice. In another embodiment of methods to generate non-human genetically modified organisms, the organism is a plant. The Class 2 CRISPR-Cas systems described herein are used to effect efficient, cost-effective gene editing and manipulation in plant cells. It is generally preferable to insert a functional recombinant DNA in a plant genome at a non-specific location. However, in certain instances, it may be useful to use site-specific integration to introduce a recombinant DNA construct into the genome. Such introduction of recombinant DNA into plants is facilitated using the Class 2 CRISPR-Cas systems of the present invention.

For embodiments in which a sesPN, a casPN, and/or a Cas polynucleotide is used to transform a plant, a promoter demonstrating the ability to drive expression of the coding sequence in that particular species of plant is selected. Promoters that can be used effectively in different plant species are well known in the art, as well. Inducible, viral, synthetic, or constitutive promoters can be used in plants for expression of polypeptides. Promoters that are spatially regulated, temporally regulated, and spatio-temporally regulated can also be useful. A list of preferred promoters includes, but is not limited to, the FMV35S promoter and the enhanced CaMV35S promoters. Plant tissue specific promoters are known in the art, for example, root-enhanced promoters, and can be used when it is preferable to achieve the highest levels of expression of these genes within a particular plant tissue, for example, the roots of plants.

In any transformation experiment, DNA is introduced into a small percentage of target cells only. Genes that encode selectable markers are useful and efficient in identifying cells that are stably transformed when they receive and integrate a transgenic DNA construct into their genomes. Preferred marker genes provide selective markers that confer resistance to a selective agent, such as an antibiotic or herbicide. Any herbicide to which plants may be resistant is a useful agent for a selective marker.

A recombinant DNA vector or construct of the present invention will typically comprise a selectable marker that confers on plant cells a selectable phenotype. Selectable markers also may be used to select for plants or plant cells containing the sesPN, casPN, and/or Cas polypeptides of the present invention. The selectable marker may encode, for example, antibiotic resistance (e.g., G418 bleomycin, kanamycin, hygromycin), biocide resistance, or herbicide resistance (e.g., glyphosate). Examples of selectable markers include, but are not limited to, a neo gene that codes for kanamycin resistance and can be selected for using kanamycin, G418, etc.; a bar gene that codes for bialaphos resistance; a mutant EPSP synthase gene that encodes glyphosate resistance; a nitrilase gene that confers resistance to bromoxynil; a mutant acetolactate synthase gene (ALS) that confers imidazolinone or sulphonylurea resistance; and a methotrexate-resistant DHFR gene.

Potentially transformed cells are exposed to the selective agent, and, among the surviving cells there will be cells in which the resistance-conferring gene has been integrated and is expressed at sufficient levels for cell survival. Cells may be tested further to confirm stable integration of the exogenous DNA.

A screenable marker, which may be used to monitor expression, may also be included in a recombinant vector or construct of the present invention. Screenable markers include, but are not limited to, a β-glucuronidase or uidA gene (GUS) that encodes an enzyme for which various chromogenic substrates are known; an R-locus gene, which encodes a product that regulates the production of anthocyanin pigments (red color) in plant tissues; a β-lactamase gene, a gene that encodes an enzyme for which various chromogenic substrates are known (e.g., PADAC, a chromogenic cephalosporin); a luciferase gene; a xylE gene that encodes a catechol dioxygenase that can convert chromogenic catechols; an α-amylase gene; a tyrosinase that encodes an enzyme capable of oxidizing tyrosine to DOPA and dopaquinone, which in turn condenses to melanin; and an α-galactosidase, which catalyzes a chromogenic α-galactose substrate.

Polynucleotides of the present invention may be introduced into a plant cell, either permanently or transiently, together with other genetic elements. These genetic elements include, but are not limited to, promoters, enhancers, introns, and untranslated leader sequences.

Among preferred plant transformation vectors are those derived from a Ti plasmid of Agrobacterium tumefaciens (Lee, L. Y., et al., “T-DNA Binary Vectors and Systems,” Plant Physiol. 2008 February; 146(2): 325-332). Also useful and known in the art are Agrobacterium rhizogenes plasmids. There are several commercial software products designed to facilitate selection of appropriate plant plasmids for plant cell transformation and gene expression in plants and methods to easily enable cloning of such polynucleotides. SnapGene™ (GSL Biotech LLC, Chicago, Ill.; www.snapgene.com/resources/plasmid_files/your_time_is_valuable/), for example, provides an extensive list of plant vectors including individual vector sequences and vector maps, as well as commercial sources for many of the vectors.

Methods and compositions for transforming plants by introducing a recombinant DNA construct into a plant genome includes any of a number of methods known in the art. One method for constructing transformed plants is microprojectile bombardment. Agrobacterium-mediated transformation is another method for constructing transformed plants. Alternatively, other non-Agrobacterium species (e.g., Rhizobium) and other prokaryotic cells that are able to infect plant cells and introduce heterologous nucleotide sequences into the infected plant cell's genome can be used. Other transformation methods include electroporation, liposomes, transformation using pollen or viruses, chemicals that increase free DNA uptake, or free DNA delivery by means of microprojectile bombardment. DNA constructs of the present invention may be introduced into the genome of a plant host using conventional transformation techniques that are well known to those skilled in the art (see, e.g., “Methods to Transfer Foreign Genes to Plants,” Y Narusaka, et al., cdn.intechopen.com/pdfs-wm/30876.pdf).

As an alternative to using a recombinant DNA construct for the direct transformation of a plant, transgenic plants can be formed by crossing a first plant that has been transformed with a recombinant DNA construct with a second plant that lacks the construct. As an example, a first plant line into which has been introduced a recombinant DNA construct for gene suppression can be crossed with a second plant line to introgress the recombinant DNA into the second plant line, thus forming a transgenic plant line.

The Class 2 CRISPR-Cas systems of the present invention provide plant breeders with a new tool to induce mutations. Accordingly, one skilled in the art can analyze the genome of sources of resistance genes and use the present invention in varieties having desired traits or characteristics to induce the rise of resistance genes; this result can be achieved with more precision than by using previous mutagenic agents, thereby accelerating and enhancing plant breeding programs.

As an alternative to expressing the components of the Class 2 CRISPR-Cas systems of the present invention, a sesPN, casPN, and cognate Cas protein can be directly introduced into a cell, for example, the three components in complex to form a nucleoprotein particle. Or one or more component can be expressed by a cell and the other component(s) directly introduced. Methods to introduce the components into a cell include electroporation, lipofection, and ballistic gene transfer (e.g., using a gene gun or a biolistic particle delivery system).

Another aspect of the present invention comprises methods of modifying DNA using sesPNs, casPNs, and Cas proteins. Generally, a method of modifying DNA involves contacting a target DNA sequence with a sesPN/casPN/Cas protein complex (a “targeting complex”). In some cases, the Cas protein component exhibits nuclease activity that cuts (cleaves) one or both strands of a target double-stranded DNA at a site in the double-stranded DNA that is complementary to a DNA target binding sequence in the sesPN. With nuclease-active Class 2 Cas proteins, site-specific cleavage of the target DNA occurs at sites determined by (i) base-pair complementarity between the DNA target binding sequence in the sesPN and the target DNA, and (ii) a protospacer adjacent motif (PAM) present in the target DNA. The nuclease activity cleaves the target DNA to produce double-strand breaks. In cells the double-strand breaks are repaired by one of two cellular mechanisms: non-homologous end joining (NHEJ), and homology-directed repair (HDR).

Repair of breaks, created by double-strand cuts, by NHEJ occurs by direct ligation of the break ends to one another. Typically, no new polynucleotide sequences are inserted at the site of the double-strand break; however, insertions or deletions may occur when a small number of nucleotides are either randomly inserted or deleted at the site of the double-strand break.

Two different sesPNs that comprise DNA target binding sequences targeting two different DNA target sequences are used to provide deletion of an intervening DNA sequence (i.e., the DNA sequence between the two DNA target sequences). Deletion of the intervening sequence occurs when NHEJ rejoins the ends of the two cleaved DNA target sequences to each other. Similarly, NHEJ may be used to direct insertion of donor template DNA or portion thereof using donor template DNA, for example, containing compatible overhangs. Accordingly, one embodiment of the present invention includes methods of modifying DNA by introducing insertions and/or deletions at a target DNA site.

Repair of breaks, created by double-strand cuts, by HDR uses a donor polynucleotide (donor template DNA) or oligonucleotide having homology to the cleaved target DNA sequence. The donor template DNA or oligonucleotide is used for repair of the double-strand break in the target DNA sequence resulting in the transfer of genetic information (i.e., polynucleotide sequences) from the donor template DNA or oligonucleotide at the site of the double-strand break in the DNA. Accordingly, new genetic information (i.e., polynucleotide sequences) may be inserted or copied at a target DNA site.

In some methods of the present invention, cells comprise polynucleotide sequences encoding a sesPN, a casPN, and a Cas protein comprising active RuvC-like and HNH nuclease domains (Class 2 Type II CRISPR-Cas systems) or an active RuvC-like nuclease domain (Class 2 Type V CRISPR-Cas systems). Expression of these polynucleotide sequences is placed under the control of one or more inducible promoter. When the DNA binding sequence of the sesPN is complementary to a DNA target in, for example, a promoter of a gene, upon inducing expression of the sesPN, casPN, and Cas protein, expression from the gene is shut off (as a result of the cleavage of the promoter sequence by the sesPN/a casPN/Cas protein complex). The polynucleotides encoding the sesPN, casPN, and Cas protein can be integrated in the cellular genome, present on vectors, or combinations thereof.

In methods of modifying a target DNA using the sesPN/casPN/Cas protein complexes of the present invention, repair of a double-stranded break by either NHEJ and/or HDR can lead to, for example, gene correction, gene replacement, gene tagging, gene disruption, gene mutation, transgene insertion, or nucleotide deletion. Methods of modifying a target DNA using the sesPN/casPN/Cas protein complexes of the present invention in combination with a donor template DNA can be used to insert or replace polynucleotide sequences in a DNA target sequence, for example, to introduce a polynucleotide that encodes a protein or functional RNA (e.g., siRNA), to introduce a protein tag, to modify a regulatory sequence of a gene, or to introduce a regulatory sequence to a gene (e.g. a promoter, an enhancer, an internal ribosome entry sequence, a start codon, a stop codon, a localization signal, or polyadenylation signal), to modify a nucleic acid sequence (e.g., introduce a mutation), and the like.

In some embodiments of the sesPN/casPN/Cas protein complexes of the present invention, a mutated form of the Cas protein is used. Modified versions of a Cas9 protein can contain a single inactive catalytic domain (i.e., either inactive RuvC or inactive HNH). Such modified Cas9 proteins cleave only one strand of a target DNA thus creating a single-strand break. Modified Cas9 protein having a single inactive catalytic domain can bind DNA based on sesPN-conferred specificity; however, it will only cut one of the double-stranded DNA strands (i.e., a nickase). As an example, in the Cas9 protein from Streptococcus pyogenes the RuvC domain can be inactivated by a D10A mutation and the HNH domain can be inactivated by an H840A mutation. When using a modified Cas protein having a single inactive catalytic domain in the sesPN/casPN/Cas protein complexes of the present invention NHEJ is less likely to occur at the single-strand break site.

In other modified versions, the Cas protein has no substantial nuclease activity (e.g., Cas 9 protein wherein both catalytic domains are inactive, i.e., inactive RuvC and inactive HNH); “dCas”. Such dCas proteins have no substantial nuclease activity; however, sesPN/casPN/dCas protein complexes can bind DNA based on sesPN-conferred specificity. As an example, in the Cas9 protein from Streptococcus pyogenes a D10A mutation and an H840A mutation result in a dCas 9 protein having no substantial nuclease activity.

In some embodiments, the Cas protein is a Cas9 protein or a Cpf1 protein. In some embodiments, the Cas protein comprises a Cas protein having modified enzymatic activity, for example, a Cas protein with reduced nuclease activity can be a nickase, i.e., it can be modified to cleave one strand of a target nucleic acid duplex. In some embodiments, a Cas protein can be modified to have no nuclease activity, i.e., it does not cleave any strand of a target nucleic acid duplex, or any single strand of a target nucleic acid. Examples of Cas proteins with reduced, or no nuclease activity can include a Cas9 with a modification to the HNH and/or RuvC nuclease domains, and a Cpf1 with a modification to the RuvC nuclease domain. Non-limiting examples of such modifications can include D917A, E1006A and D1225A to the RuvC nuclease domain of the F. novicida Cpf1 and alteration of residues D10, G12, G17, E762, H840, N854, N863, H982, H983, A984, D986, and/or A987 of the S. pyogenes Cas9, and their corresponding amino acid residues in other Cpf1 and Cas9 proteins.

The present invention also includes a detectable label, including a moiety that can provide a detectable signal, attached to one or more of a sesPN, a casPN, or a Cas protein (e.g., a dCas protein) of a sesPN/casPN/Cas protein complex. Examples of detectable labels include, but are not limited to, an enzyme, a radioisotope, a member of a specific binding pair, a fluorophore (FAM), a fluorescent protein (green fluorescent protein, red fluorescent protein, mCherry, tdTomato), an DNA or RNA aptamer together with a suitable fluorophore (enhanced GFP (EGFP), “Spinach”), a quantum dot, an antibody, and the like. A large number and variety of suitable detectable labels are well-known to one of ordinary skill in the art.

In one aspect, the present invention relates to a composition comprising a Class 2 CRISPR-Cas associated polynucleotide lacking a spacer element (casPN), wherein the casPN is capable of associating with (i) a Class 2 CRISPR-Cas protein and (ii) a distinct spacer element sequence polynucleotide (sesPN) comprising a target nucleic acid binding sequence, thereby forming a Class 2 CRISPR-Cas nucleoprotein complex. This Class 2 CRISPR-Cas nucleoprotein complex is capable of site-directed binding to a target nucleic acid complementary to the target nucleic acid binding sequence of the sesPN. A different embodiment of the present invention includes a composition comprising a Class 2 CRISPR-Cas associated polynucleotide lacking a spacer element (casPN), wherein the casPN is capable of associating with a Class 2 CRISPR-Cas protein to form a casPN/Cas nucleoprotein complex, and the associating forms a nucleic acid sequence binding channel in the casPN/Cas protein complex capable of binding a nucleic acid sequence. In related aspects kits comprise such compositions and, for example, a buffer.

In one embodiment the present invention includes a method of binding a target nucleic acid, comprising contacting a nucleic acid comprising the target nucleic acid with a Class 2 CRISPR-Cas nucleoprotein complex comprising an sesPN comprising a target nucleic acid binding sequence, a casPN, and a Cas protein, thereby facilitating binding of the complex to the target nucleic acid. In an additional embodiment the present invention includes a method of cutting a target nucleic acid, comprising contacting a nucleic acid comprising the target nucleic acid with a Class 2 CRISPR-Cas nucleoprotein complex comprising a sesPN comprising a target nucleic acid binding sequence, a casPN, and a Cas protein, thereby facilitating binding of the Class 2 CRISPR-Cas nucleoprotein complex to the target nucleic acid, wherein the bound Class 2 CRISPR-Cas nucleoprotein complex cuts the target nucleic acid. Such methods of binding a target nucleic acid or cutting a target nucleic acid are carried out in vitro, in cell (e.g., in cultured cells), ex vivo (e.g., stem cells removed from a subject), and in vivo.

The present invention also includes methods of modulating in vitro or in vivo transcription using sesPN/casPN/Cas protein complexes described herein. In one embodiment, a sesPN/casPN/dCas protein complex can repress gene expression by interfering with transcription when the sesPN directs DNA target binding of the sesPN/casPN/dCas protein complex to the promoter region of the gene. Use of sesPN/casPN/dCas protein complexes to reduce transcription also includes complexes wherein the dCas protein is fused to a known down regulator of a target gene (e.g., a repressor polypeptide). For example, expression of a gene is under the control of regulatory sequences to which a repressor polypeptide can bind. A sesPN can direct DNA target binding of a sesPN/casPN/dCas-repressor protein complex to the DNA sequences encoding the regulatory sequences or adjacent the regulatory sequences such that binding of the sesPN/casPN/dCas-repressor protein complex brings the repressor protein into operable contact with the regulatory sequences. Similarly, dCas9 is fused to an activator polypeptide to activate or increase expression of a gene under the control of regulatory sequences to which an activator polypeptide can bind.

Another method of the present invention is the use of a sesPN/casPN/dCas protein complex in methods to isolate or purify regions of genomic DNA (gDNA). In an embodiment of the method, a dCas protein is fused to an epitope (e.g., a FLAG® (Sigma Aldrich, St. Louis, Mo.) epitope) or an anti-Cas protein antibody is used and a sesPN directs DNA target binding of a sesPN/casPN/dCas protein-epitope complex to DNA sequences within the region of genomic DNA to be isolated or purified. An affinity agent is used to bind the epitope and the associated gDNA bound to the sesPN/casPN/dCas protein-epitope complex.

In another aspect the present invention relates to an in vitro, in cell, ex vivo, or in vivo method of modifying genomic DNA in a cell. The method comprises contacting a target DNA sequence in the genomic DNA with a Class 2 Type II CRISPR-Cas system, the system comprising a casPN, a sesPN, and a Cas protein, wherein the casPN, the Cas protein, and the sesPN form a complex that binds to the target DNA sequence resulting in a modification of the target DNA sequence in the genomic DNA of the cell. A donor polynucleotide is an addition to the system in some embodiments. Such modifications of the target DNA sequence in the genomic DNA include, but are not limited to, deletions, insertions, substitutions, missense mutations, nonsense mutations, frameshift mutations, substitution of one or more amino acids encoded by a coding sequence of the target DNA, as well as combinations thereof. Examples of host cells that can be modified by this method are discussed above. In some embodiments, the present invention includes cells made by this method.

The Class 2 CRISPR-Cas sesPN, casPN, and Cas proteins of the present invention are useful in CRISPR-related methods, vectors, and applications known to those of ordinary skill in the art in view of the guidance of the present specification.

In further aspect, the present invention includes kits comprising a casPN or polynucleotides encoding a casPN. Kits can comprise one or more of the following: a casPN and cognate Cas protein; polynucleotides encoding a casPN and cognate Cas protein; recombinant cells comprising a casPN; recombinant cells comprising a casPN and cognate Cas protein; and the like. Kits can also include a sesPN or polynucleotides encoding a sesPN. In further aspect, the present invention includes kits to carry out the methods of the present invention, the kits comprising a casPN or polynucleotides encoding a casPN. Such kits can also include a sesPN or polynucleotides encoding a sesPN. Any kits of the present invention can further comprise other components such as solutions, buffers, substrates, cells, instructions, vectors (e.g., targeting vectors), and so on.

The present invention also includes pharmaceutical compositions comprising a sesPN, a casPN, and a Cas protein, or one or more polynucleotides encoding a sesPN, a casPN, and a Cas protein. Pharmaceutical compositions may further comprise pharmaceutically acceptable vehicles.

The Class 2 CRISPR-Cas systems of the present invention as described herein provide a number of advantages including, but not limited to, the following:
increased binding affinity of sesPN and/or casPN to a Cas protein using covalent cross-linking or tethering of sesPN and/or casPN to a Cas protein versus Cas9 tracrRNA/crRNA, Cas9 sgRNA, or Cpf1 crRNA charge-based interaction with Cas protein;
provision of an activatable system (e.g., when an sesPN comprises UV cross-linking or thiol cross-linking moieties, or the Csy4 RNA hairpin comprises a riboswitch activatable by, for example, a small molecule);
resistance to RNase degradation provided by modified thiol-linkages in sesRNA or casRNA;
fast generation of screening, e.g., screens can be developed by creating a Csy4-sesPN library and pairing each sesPN of the library with the same casPN and (dCsy4)-Cas protein for screening; and
improved cell delivery of sesPN into cells expressing casPN and Cas protein (versus delivery of crRNA into cells expressing tracrRNA and Cas protein) due to the smaller size of the sesPN.

Further aspects of the present invention include, but are not limited to, the following. These aspects are sequentially numbered for ease of reference.

A first aspect of the present invention is a Class 2 Type II CRISPR-Cas system comprising a casPN and a sesPN. In one embodiment of the first aspect, the Class 2 Type II CRISPR-Cas system comprises a first polynucleotide (casPN) and a second polynucleotide (sesPN). In the system, the first polynucleotide (casPN) comprises a tracr element (as described herein there is the proviso that the first polynucleotide (casPN) does not contain a target nucleic acid binding sequence (e.g., a target DNA sequence)). When the tracr element complexes with a Cas protein the Cas protein more preferentially binds DNA sequences containing protospacer adjacent motif (PAM) sequences than DNA sequences without PAM sequences. The second polynucleotide (sesPN) comprises the target nucleic acid binding sequence with the provisos that (i) the second polynucleotide (sesPN) comprises RNA or DNA, (ii) the first polynucleotide (casPN) and second polynucleotide (sesPN) are separate polynucleotides, and (iii) the first polynucleotide (casPN) and second polynucleotide (sesPN) do not interact through base-pair hydrogen bonding. In one embodiment, the sesPN does not form base-pair hydrogen bonds with the casPN to form a stable secondary structure. In another embodiment, the sesPN does not interact with the casPN in the absence of a Cas protein. In yet a further embodiment, the casPN is capable of interacting with a cognate Cas protein and a sesPN to form a sesPN/casPN/Cas protein nucleoprotein complex, wherein the binding of casPN to the Cas protein activates the complex for sesPN-guided target nucleic acid binding (e.g., target DNA binding).

A second aspect of the present invention is a Class 2 Type II CRISPR-Cas system comprising a casPN and a sesPN. In one embodiment the Class 2 Type II CRISPR-Cas system comprises a first polynucleotide (casPN) and a second polynucleotide (sesPN). In the system, a first polynucleotide (casPN) has a 5′ end and a 3′ end. The first polynucleotide (casPN) comprises a first stem element and a nexus element wherein the nexus element is located 3′ of the first stem element (as described herein there is the proviso that the first polynucleotide (casPN) does not contain a target nucleic acid binding sequence (e.g., a target DNA sequence)). A second polynucleotide (sesPN) has a 5′ end and a 3′ end. The second polynucleotide (sesPN) comprises a target nucleic acid binding sequence (e.g., a target DNA binding sequence), with the provisos that (i) the second polynucleotide (sesPN) comprises RNA or DNA, (ii) the first polynucleotide (casPN) and second polynucleotide (sesPN) are separate polynucleotides, and (iii) the second polynucleotide (sesPN) does not form part of the first stem element of the first polynucleotide (casPN).

In one embodiment of the second aspect of the invention, the first polynucleotide (casPN) further comprises, in a 5′ to 3′ direction, a first lower stem sequence, a first bulge sequence, a first upper stem sequence, a loop sequence, a second upper stem sequence wherein the first upper stem sequence and the second upper stem sequence form an upper stem element by base-pair hydrogen bonding between the first upper stem sequence and the second upper stem sequence, a second bulge sequence, a second lower stem sequence wherein the first lower stem sequence and second lower stem sequence form the first stem element by base-pair hydrogen bonding between the first lower stem sequence and second lower stem sequence. In another embodiment of the second aspect of the invention, the first polynucleotide (casPN) further comprises, in a 5′ to 3′ direction, a first stem sequence, a loop sequence, and a second stem sequence wherein the first stem sequence and the second stem sequence form a first stem element by base-pair hydrogen bonding between the first stem sequence and the second stem sequence.

In further embodiments of the second aspect of the invention, the sesPN does not form base-pair hydrogen bonds with polynucleotides of the casPN that form the first stem. In another embodiment, the sesPN does not interact with the casPN in the absence of a Cas protein. In yet a further embodiment, the casPN is capable of interacting with a cognate Cas protein and a sesPN to form a sesPN/casPN/Cas protein nucleoprotein complex, wherein the binding of casPN to the Cas protein activates the complex for sesPN-guided target nucleic acid binding (e.g., target DNA binding). In an additional embodiment, the casPN further comprises a tracr element.

Further embodiments of the first and second aspects of the present invention include the following:

wherein the second polynucleotide (sesPN) further comprises one or more affinity sequence located: 5′ to the target nucleic acid binding sequence (e.g., target DNA binding sequence), 3′ to the target nucleic acid binding sequence (e.g., target DNA binding sequence), or both 5′ and 3′ to the target nucleic acid binding sequence (e.g., target DNA binding sequence);

wherein the affinity sequence further comprises one or more cross-linking moiety located 5′ to the target nucleic acid binding sequence (e.g., target DNA binding sequence), 3′ to the target nucleic acid binding sequence (e.g., target DNA binding sequence), both 5′ and 3′ to the target nucleic acid binding sequence (e.g., target DNA binding sequence), or within the target nucleic acid binding sequence (e.g., target DNA binding sequence). In some embodiments the one or more cross-linking moiety is a photoactive nucleotide (e.g., 6-Thio-dG or 4-Thiothymidine);

wherein the affinity sequence further comprises one or more ligand binding moiety located 5′ to the target nucleic acid binding sequence (e.g., target DNA binding sequence), 3′ to the target nucleic acid binding sequence (e.g., target DNA binding sequence), or both 5′ and 3′ to the target nucleic acid binding sequence (e.g., target DNA binding sequence). In some embodiments the ligand binding moiety is an aptamer, a biotin, an estradiol, a rapamycin, a FK506 molecule, or a zinc finger domain coding sequence;

wherein the first polynucleotide (casPN) comprises RNA bases, DNA bases, or a combination of RNA bases and DNA bases;

wherein the first polynucleotide (casPN) is RNA and is encoded by a first DNA coding sequence, and the second polynucleotide (sesPN) is chemically synthesized; and

wherein the Class 2 Type II CRISPR-Cas system further comprises a third polynucleotide encoding a Cas protein.

A third aspect of the present invention relates to compositions comprising the polynucleotides of the first and second aspects of the invention. In some embodiments such compositions comprises a cognate Cas protein or a polynucleotide encoding a cognate Cas protein.

A fourth aspect of the invention relates to methods of manufacturing the polynucleotides of the first and second aspects of the invention. In one embodiment, the method of manufacturing comprises chemically synthesizing a first polynucleotide (casPN), a second polynucleotide (sesPN), or both the first polynucleotide (casPN) and the second polynucleotide (sesPN), wherein the first polynucleotide (casPN) comprises RNA bases, DNA bases, or a combination of RNA bases and DNA bases and the second polynucleotide (sesPN) comprises RNA bases or DNA bases.

A fifth aspect of the invention relates to one or more expression cassette comprising a casDNA and a sesPN. In one embodiment, one or more expression cassette comprises a first DNA sequence encoding a first polynucleotide (casDNA) and a second DNA sequence encoding a second polynucleotide (sesRNA), wherein the first DNA sequence comprises a transcription promoter and a reverse transcriptase primer operably linked to the first polynucleotide (casDNA), and the second DNA sequence comprises a transcription promoter operably linked to the second polynucleotide (sesRNA). In one embodiment, the one or more expression cassette further comprises an expression cassette comprising a third DNA sequence comprising a transcription promoter and a translational regulatory sequence operably linked to a Cas protein coding sequence. Furthermore, expression vectors can comprise the one or more expression cassettes. In some embodiments, recombinant cells comprise the one or more expression cassettes. Such recombinant cells can transcribe the second polynucleotide (sesRNA) from the second DNA sequence and transcribe the first DNA sequence to create a RNA that is reverse transcribed to generate the first polynucleotide (casDNA). In addition, a Cas protein can be expressed in the recombinant cells. Examples of cells useful in the practice of this aspect of the present invention include, but are not limited to, a plant cell, a yeast cell, a bacterial cell, an algal cell, or a mammalian cell.

A sixth aspect of the invention relates to one or more expression cassette comprising a casRNA and a sesRNA. In one embodiment, one or more expression cassette comprises a first DNA sequence encoding a first polynucleotide (casRNA) and a second DNA sequence encoding a second polynucleotide (sesRNA), wherein the first DNA sequence comprises a transcription promoter operably linked to the first polynucleotide (casRNA), and the second DNA sequence comprises a transcription promoter operably linked to the second polynucleotide (sesRNA). In one embodiment, the one or more expression cassette further comprises an expression cassette comprising a third DNA sequence comprising a transcription promoter and a translational regulatory sequence operably linked to a Cas protein coding sequence. Furthermore, expression vectors can comprise the one or more expression cassettes. In some embodiments, recombinant cells comprise the one or more expression cassettes. Such recombinant cells can transcribe a first polynucleotide (casRNA) from a first DNA sequence and transcribe a second polynucleotide (sesRNA) from a second DNA sequence. In addition, a Cas protein can be expressed in the recombinant cells. In the recombinant cells, an expression cassette can be integrated, or an expression vector can comprise an expression cassette, or combinations thereof. In one embodiment, an expression cassette comprising a first DNA sequence encoding a first polynucleotide (casRNA) is integrated at a site in genomic DNA of the recombinant cell, and an expression cassette comprising a third DNA sequence comprising the transcription promoter and the translational regulatory sequence operably linked to a Cas protein coding sequence is integrated at a site in genomic DNA of the recombinant cell. Examples of cells useful in the practice of this aspect of the present invention include, but are not limited to, a plant cell, a yeast cell, a bacterial cell, an algal cell, or a mammalian cell.

A seventh aspect of the invention relates to a kit comprising the first polynucleotide (casPN) and the second polynucleotide (sesPN) of the Class 2 Type II CRISPR-Cas system of the first and second aspects of the invention. In one embodiment the kit further comprises a Cas protein. In another embodiment the kit comprises a Cas protein complexed to a casPN. In some embodiments, kits comprise one or more expression cassettes comprising a first DNA sequence encoding the first polynucleotide (casPN) and a second DNA sequence encoding the second polynucleotide (sesPN). Kits can further comprise an expression cassette comprising a third DNA sequence encoding a Cas protein. In some embodiments, the kits comprise one or more expression vectors having the expression cassettes.

An eighth aspect of the present invention is a Type II CRISPR-Cas tracr element comprising a casPN. In one embodiment of the eighth aspect, the Class 2 Type II CRISPR-Cas system comprises a first polynucleotide (casPN). The first polynucleotide (casPN) comprises a tracr element (as described herein there is the proviso that the first polynucleotide (casPN) does not contain a target nucleic acid binding sequence (e.g., a target DNA sequence)). When the tracr element complexes with a Cas protein the Cas protein more preferentially binds DNA sequences containing PAM sequences than DNA sequences without PAM sequences. In one embodiment, a sesPN does not form base-pair hydrogen bonds with the casPN to form a stable secondary structure. In another embodiment, a sesPN does not interact with the casPN in the absence of a Cas protein. In yet a further embodiment, the casPN is capable of interacting with a cognate Cas protein and a sesPN to form a sesPN/casPN/Cas protein nucleoprotein complex, wherein the binding of casPN to the Cas protein activates the complex for sesPN-guided DNA target binding.

A ninth aspect of the present invention is a Type II CRISPR-Cas associated polynucleotide. In one embodiment a first polynucleotide (casPN) has a 5′ end and a 3′ end. The first polynucleotide (casPN) comprises a first stem element and a nexus element wherein the nexus element is located 3′ of the first stem element (as described herein there is the proviso that the first polynucleotide (casPN) does not contain a target nucleic acid binding sequence (e.g., a target DNA sequence)).

In one embodiment of the ninth aspect of the invention, the first polynucleotide (casPN) further comprises, in a 5′ to 3′ direction, a first lower stem sequence, a first bulge sequence, a first upper stem sequence, a loop sequence, a second upper stem sequence wherein the first upper stem sequence and the second upper stem sequence form an upper stem element by base-pair hydrogen bonding between the first upper stem sequence and the second upper stem sequence, a second bulge sequence, a second lower stem sequence wherein the first lower stem sequence and second lower stem sequence form the first stem element by base-pair hydrogen bonding between the first lower stem sequence and second lower stem sequence. In another embodiment of the ninth aspect of the invention, the first polynucleotide (casPN) further comprises, in a 5′ to 3′ direction, a first stem sequence, a loop sequence, and a second stem sequence wherein the first stem sequence and the second stem sequence form a first stem element by base-pair hydrogen bonding between the first stem sequence and the second stem sequence.

In further embodiments of the ninth aspect of the invention, a sesPN does not form base-pair hydrogen bonds with polynucleotides of the casPN that form the first stem. In another embodiment, a sesPN does not interact with the casPN in the absence of a Cas protein. In yet a further embodiment, a casPN is capable of interacting with a cognate Cas protein and a sesPN to form a sesPN/casPN/Cas protein nucleoprotein complex, wherein binding of the casPN to Cas activates the complex for sesPN-guided DNA target binding. In an additional embodiment, the casPN further comprises a tracr element.

Further embodiments of the eighth and ninth aspects of the present invention include the following:

wherein the first polynucleotide (casPN) comprises RNA bases, DNA bases, or a combination of RNA bases and DNA bases;

wherein the first polynucleotide (casPN) is DNA; and

wherein the first polynucleotide (casPN) is RNA.

A tenth aspect of the present invention relates to methods of manufacturing a first polynucleotide of the eighth and ninth aspects of the present invention, comprising chemically synthesizing the first polynucleotide

An eleventh aspect of the present invention relates to compositions comprising a first polynucleotide (casPN) of the eighth and ninth aspects of the invention.

A twelfth aspect of the present invention relates to expression cassettes comprising a casRNA. In one embodiment, an expression cassette comprises a first DNA sequence encoding a first polynucleotide (casRNA) wherein the first DNA sequence comprises a transcription promoter operably linked to the first polynucleotide (casRNA). In one embodiment, an expression cassette comprising a second DNA sequence comprising a transcription promoter and a translational regulatory sequence operably linked to a Cas protein coding sequence is present in the expression cassette or in a separate expression cassette. Furthermore, expression vector(s) can comprise the expression cassette(s). In some embodiments, recombinant cells comprise the expression vector(s). In some embodiments, recombinant cells comprise the expression cassette(s). Recombinant cells, comprising these expression vector(s) or expression cassette(s), can transcribe the first polynucleotide (casRNA) from the first DNA sequence. In addition, a Cas protein can be expressed in the recombinant cells. In the recombinant cells, an expression cassette can be integrated, or an expression vector can comprise an expression cassette, or combinations thereof. In one embodiment, an expression cassette comprising a first DNA sequence encoding a first polynucleotide (casRNA) is integrated at a site in genomic DNA of the recombinant cell, and an expression cassette comprising a second DNA sequence comprising a transcription promoter and a translational regulatory sequence operably linked to a Cas protein coding sequence is integrated at a site in genomic DNA of the recombinant cell. Examples of cells useful in the practice of this aspect of the present invention include, but are not limited to, a plant cell, a yeast cell, a bacterial cell, an algal cell, or a mammalian cell.

A thirteenth aspect of the invention relates to a kit comprising a first polynucleotide (casPN) of the eighth and ninth aspects of the invention. In one embodiment the kit further comprises a Cas protein. In another embodiment the kit comprises a Cas protein complexed to a casPN. In some embodiments, kits comprise one or more expression cassettes comprising a first DNA sequence encoding the first polynucleotide (casPN) and a second DNA sequence encoding a Cas protein. In some embodiments, the kits comprise one or more expression vectors having the expression cassettes.

A fourteenth aspect of the present invention relates to an in vivo method of modifying genomic DNA in a eukaryotic cell. The method comprises contacting a target DNA sequence in the genomic DNA with a Class 2 Type II CRISPR-Cas system. The system comprising a casPN, a sesPN, and a Cas protein, wherein the casPN, the Cas protein, and the sesPN form a complex that binds to the target DNA sequence resulting in a modification of the target DNA sequence.

In one embodiment, the in vivo method of modifying genomic DNA in a eukaryotic cell comprises contacting a target DNA sequence in the genomic DNA with a Class 2 Type II CRISPR-Cas system comprising:

a first polynucleotide (casPN) comprising a tracr element (as described herein there is the proviso that the first polynucleotide (casPN) does not contain a target nucleic acid binding sequence (e.g., a target DNA sequence)), wherein when the tracr element complexes with a Cas protein the Cas protein more preferentially binds DNA sequences containing PAM sequences than DNA sequences without PAM sequences;

a second polynucleotide (sesPN) comprising a target nucleic acid binding sequence (e.g., target DNA binding sequence) with the provisos that (i) the second polynucleotide (sesPN) is a RNA, (ii) the first polynucleotide (casPN) and second polynucleotide (sesPN) are separate polynucleotides, and (iii) the first polynucleotide (casPN) and second polynucleotide (sesPN) do not interact through base-pair hydrogen bonding, and

a Cas protein;

wherein the first polynucleotide (casPN), the Cas protein, and the second polynucleotide (sesPN) form a complex that binds to the target DNA sequence resulting in a modification of the target DNA sequence. In another embodiment the method further comprising contacting the target DNA sequence in the genomic DNA with a donor template DNA wherein the modification is formed via homology-directed repair (HDR) in a eukaryotic cell and at least a portion of a donor template DNA is integrated at the target DNA sequence. In a further embodiment, the modification is formed by inserting DNA using non-homologous end joining (NHEJ). In one embodiment of the method, the modification is a deletion or insertion formed via NHEJ in a eukaryotic cell. In some embodiments, a targeting vector comprises the donor template DNA. In other embodiments the donor template DNA is a double-stranded oligomer.

In another embodiment of an in vivo method of modifying genomic DNA in a eukaryotic cell, the method comprises contacting a target DNA sequence in the genomic DNA with a Class 2 Type II CRISPR-Cas system comprising:

a first polynucleotide (casPN), having a 5′ end and a 3′ end, the first polynucleotide comprising a first stem element and a nexus element wherein the nexus element is located 3′ of the first stem element (as described herein there is the proviso that the first polynucleotide (casPN) does not contain a target nucleic acid binding sequence (e.g., a target DNA sequence)); and

a second polynucleotide (sesPN), having a 5′ end and a 3′ end, comprising a target nucleic acid binding sequence (e.g., target DNA binding sequence), with the provisos that (i) the second polynucleotide (sesPN) is a RNA, (ii) the first polynucleotide (casPN) and second polynucleotide (sesPN) are separate polynucleotides, and (iii) the second polynucleotide (sesPN) does not form part of the first stem element of the first polynucleotide, and

a Cas protein;

wherein the first polynucleotide (casPN), the Cas protein, and the second polynucleotide (sesPN) form a complex that binds to the target DNA sequence resulting in a modification of the target DNA sequence.

In a fifteenth aspect the present invention relates to a method of modulating the expression of a gene comprising transcriptional regulatory elements. The method comprises contacting a target DNA sequence in the transcriptional regulatory elements of the gene with a Class 2 Type II CRISPR-Cas system comprising a casPN, a sesPN, and a Cas protein, wherein the casPN, the Cas protein, and the sesPN form a complex that binds to the target DNA sequence resulting in modulation of the expression of the gene. In one embodiment the method comprises contacting a target DNA sequence in the transcriptional regulatory elements with a Class 2 Type II CRISPR-Cas system. The system comprising:

a first polynucleotide (casPN) comprising a tracr element (as described herein there is the proviso that the first polynucleotide (casPN) does not contain a target nucleic acid binding sequence (e.g., a target DNA sequence)), wherein when the tracr element complexes with a Cas protein the Cas protein more preferentially binds DNA sequences containing PAM sequences than DNA sequences without PAM sequences;

a second polynucleotide (sesPN) comprising a target nucleic acid binding sequence (e.g., target DNA binding sequence) with the provisos that (i) the second polynucleotide (sesPN) is a RNA or DNA, (ii) the first polynucleotide (casPN) and second polynucleotide (sesPN) are separate polynucleotides, and (iii) the first polynucleotide (casPN) and second polynucleotide (sesPN) do not interact through base-pair hydrogen bonding, and

a Cas protein;

wherein the first polynucleotide (casPN), the Cas protein, and the second polynucleotide (sesPN) form a complex that binds to the target DNA sequence resulting in modulation of the expression of the gene. In one embodiment, the Cas protein is a Cas protein that is nuclease-deficient (dCas). In one embodiment, expression of a gene is under the control of regulatory sequences to which a repressor polypeptide can bind. A sesPN can direct DNA target binding of a sesPN/casPN/dCas-repressor protein complex to the DNA sequences encoding the regulatory sequences or adjacent the regulatory sequences such that binding of the sesPN/casPN/dCas-repressor protein complex brings the repressor protein into operable contact with the regulatory sequences. In another embodiment, dCas is fused to an activator polypeptide to activate or increase expression of a gene under the control of regulatory sequences to which an activator polypeptide can bind.

In another embodiment of a method of modulating the expression of a gene comprising transcriptional regulatory elements. The method comprises contacting a target DNA sequence in the transcriptional regulatory elements with a Class 2 Type II CRISPR-Cas system comprising:

a first polynucleotide (casPN), having a 5′ end and a 3′ end, the first polynucleotide (casPN) comprising a first stem element and a nexus element wherein the nexus element is located 3′ of the first stem element (as described herein there is the proviso that the first polynucleotide (casPN) does not contain a target nucleic acid binding sequence (e.g., a target DNA sequence));

a second polynucleotide (sesPN), having a 5′ end and a 3′ end, comprising a target nucleic acid binding sequence (e.g., target DNA binding sequence), with the provisos that (i) the second polynucleotide (sesPN) is a RNA or DNA, (ii) the first polynucleotide (casPN) and second polynucleotide (sesPN) are separate polynucleotides, and (iii) the second polynucleotide (sesPN) does not form part of the first stem element of the first polynucleotide (casPN), and

a Cas protein;

wherein the first polynucleotide (casPN), the Cas protein, and the second polynucleotide (sesPN) form a complex that binds to the target DNA sequence resulting in modulation of the expression of the gene. In one embodiment, the Cas protein is a Cas protein, for example Cas9, that is nuclease-deficient (dCas). In one embodiment, expression of a gene is under the control of regulatory sequences to which a repressor polypeptide can bind. A sesPN can direct DNA target binding of a sesPN/casPN/dCas-repressor protein complex to the DNA sequences encoding the regulatory sequences or adjacent the regulatory sequences such that binding of the sesPN/casPN/dCas-repressor protein complex brings the repressor protein into operable contact with the regulatory sequences. In another embodiment, dCas is fused to an activator polypeptide to activate or increase expression of a gene under the control of regulatory sequences to which an activator polypeptide can bind.

While preferred embodiments of the present invention have been shown and described herein, it will be obvious to those skilled in the art that such embodiments are provided by way of example only. From the above description and the following Examples, one skilled in the art can ascertain essential characteristics of this invention, and without departing from the spirit and scope thereof, can make changes, substitutions, variations, and modifications of the invention to adapt it to various usages and conditions. Such changes, substitutions, variations, and modifications are also intended to fall within the scope of the present disclosure.

### EXPERIMENTAL

Aspects of the present invention are further illustrated in the following Examples. Efforts have been made to ensure accuracy with respect to numbers used (e.g., amounts, concentrations, percent changes, etc.) but some experimental errors and deviations should be accounted for. Unless indicated otherwise, temperature is in degrees Centigrade and pressure is at or near atmospheric. It should be understood that these Examples, while indicating some embodiments of the invention, are given by way of illustration only.

The following examples are not intended to limit the scope of what the inventors regard as various aspects of the present invention.

Materials and Methods

Oligonucleotide sequences (e.g., sesDNA-AAVS1, sesRNA-AAVS1, and primer sequences) were provided to commercial manufacturers for synthesis (Integrated DNA Technologies, Coralville, Iowa; or Eurofins, Luxembourg, Luxembourg).

### Example 1

### Production of RNA Components

Some RNA components were produced by in vitro transcription (e.g., T7 Quick High Yield RNA Synthesis Kit, New England Biolabs, Ipswich, Mass.) from double-stranded DNA templates incorporating a T7 promoter at the 5′ end of the DNA sequences.

The double-stranded DNA templates for the specific RNA components used in the examples were assembled by PCR using 3′ overlapping primers containing the corresponding DNA sequences to the RNA components. The oligonucleotide sequences of the overlapping primers were as presented in Table 1.

TABLE 1
Overlapping Primers* for Generation of DNA Templates for
Transcription of RNA

| Type of RNA | | SEQ ID |
|---|---|---|
| Component | Target for DNA-binding Sequence | NOs. |
| sgRNA-AAVS | AAVS-1 (adeno-associated virus | 3, 4, 5, 6, 7 |
| | integration site 1 - human genome) | |
| casRNA-1 | n/a | 3, 12, 14, 15,16 |
| tracrRNA | n/a | 3, 11, 13, 15,16 |
| crRNA-AAVS | AAVS-1 | 3. 8, 9, 10 |

*DNA primer sequences are shown in FIG. 8

The DNA primers were present at a concentration of 2 nM each. Two outer DNA primers corresponding to the T7 promoter (forward primer: SEQ ID NO. 3, Table 1) and the 3′ end of the RNA sequence (reverse primers: SEQ ID NO. 7, SEQ ID NO. 16, and SEQ ID NO. 10) were used at 640 nM to drive the amplification reaction. PCR reactions were performed using KAPA HiFi Hot Start Polymerase (Kapa Biosystems, Inc., Wilmington, Mass.) and contained 0.5 units of polymerase, lx reaction buffer, and 0.4 mM dNTP. PCR assembly reactions were carried out using the following thermal cycling conditions: 95° C. for 2 minutes, 30 cycles of 20 seconds at 98° C., 20 seconds at 62° C., 20 seconds at 72° C., and a final extension at 72° C. for 2 minutes. DNA quality was evaluated by agarose gel electrophoresis (1.5%, SYBR® Safe, Life Technologies, Grand Island, N.Y.).

Between 0.25-0.5 mg of the DNA template for a Cas RNA component was transcribed using T7 Quick High Yield RNA Synthesis Kit (New England Biolabs, Ipswich, Mass.) for ˜16 hours at 37° C. Transcription reaction were DNAse I treated (New England Biolabs, Ipswich, Mass.) and purified using GeneJet RNA cleanup and concentration kit (Life Technologies, Grand Island, N.Y.), RNA yield was quantified using the Nanodrop™ 2000 system (Thermo Scientific, Wilmington Del.). The quality of the transcribed RNA was checked by agarose gel electrophoresis (2%, SYBR® Safe, Life Technologies, Grand Island, N.Y.).

The casRNA-1 sequence was as follows: 5′-GUCUCAGAGC UAUGCUGUCC UGGAAACAGG ACAGCAUAGC AAGUUGAGAU AAGGCUAGUC CGUUAUCAAC UUGAAAAAGU GGCACCGAGU CGGUGCUU-3′ (SEQ ID NO. 19).

This method for production of casRNA-1 can be applied to the production of other casRNAs as described herein.

### Example 2

### Production of Double-Stranded DNA Target Regions for Use in Cas Cleavage Assays

Double-stranded DNA target regions (e.g., AAVS-1) for biochemical assays were amplified by PCR from phenol-chloroform prepared human cell line K562 (ATCC, Manassas, Va.) genomic DNA (gDNA). PCR reactions were set up with KAPA HiFi Hot Start polymerase and contained 0.5 U of Polymerase, 1× reaction buffer, and 0.4 mM dNTPs. 20 ng/mL gDNA in a final volume of 25 μL were used to amplify the target region under the following conditions: 95° C. for 2 minutes, 4 cycles of 20 s at 98° C., 20 s at 70° C., (−2° C./cycle), 20 s at 72° C., followed by 25 cycles of 20 s at 98° C., 20 s at 62° C., 20 s at 72° C., and a final extension at 72° C. for 2 minutes. PCR products were cleaned up using Spin Smart™ PCR purification tubes (Denville Scientific, South Plainfield N.J.) and quantified using Nanodrop™ 2000 UV-Vis spectrophotometer (Thermo Scientific, Wilmington Del.).

The forward and reverse primers used for amplification of AAVS-1 from gDNA were as follows: SEQ ID NO. 17 and SEQ ID NO. 18 (FIG. 8). The amplified double-stranded DNA target for AAVS-1 was 495 bp.

Other suitable double-stranded DNA target regions are obtained using essentially the same method. For non-human target regions, genomic DNA from the selected organism (e.g., plant, bacteria, yeast, algae) is used instead of DNA derived from human cells. Furthermore, polynucleotide sources other than genomic DNA can be used (e.g., vectors and gel isolated DNA fragments).

### Example 3

### Cas Cleavage Assays

This example illustrates the use of in vitro Cas cleavage assays to evaluate and compare the percent cleavage of selected Cas protein/polynucleotide nucleoprotein complexes relative to selected double-stranded DNA target sequences.

The cleavage of double-stranded DNA target sequences was determined for sgRNA-AAVS, tracrRNA/crRNA-AAVS, casRNA-1/sesRNA-AAVS1, and casRNA-1/sesDNA-AAVS1 of Example 2 against a double-stranded DNA target (AAVS-1).

The sgRNA-AAVS, tracrRNA/crRNA-AAVS, casRNA-1/sesDNA-AAVS, or casRNA-1/sesRNA-AAVS1 were mixed, and incubated for 2 minutes at 95° C., removed from thermocycler and allowed to equilibrate to room temperature. For the tracrRNA/crRNA-AAVS, casRNA-1/sesDNA-AAVS1, and casRNA-1/sesRNA-AAVS1 each component was present in equimolar amounts.

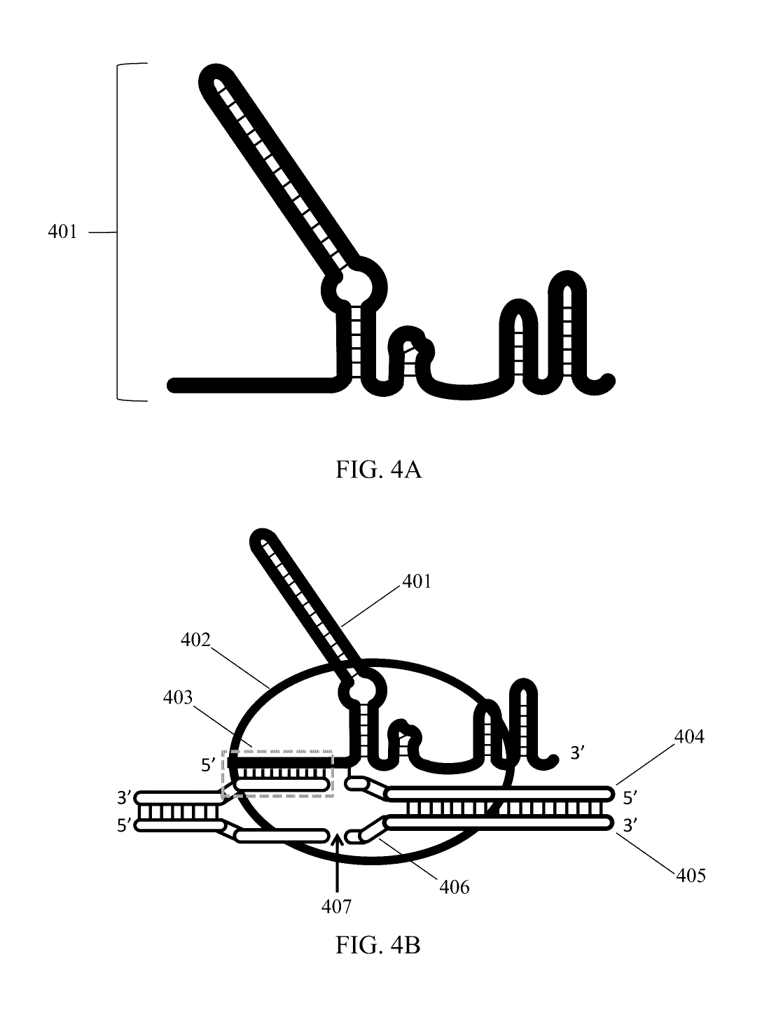

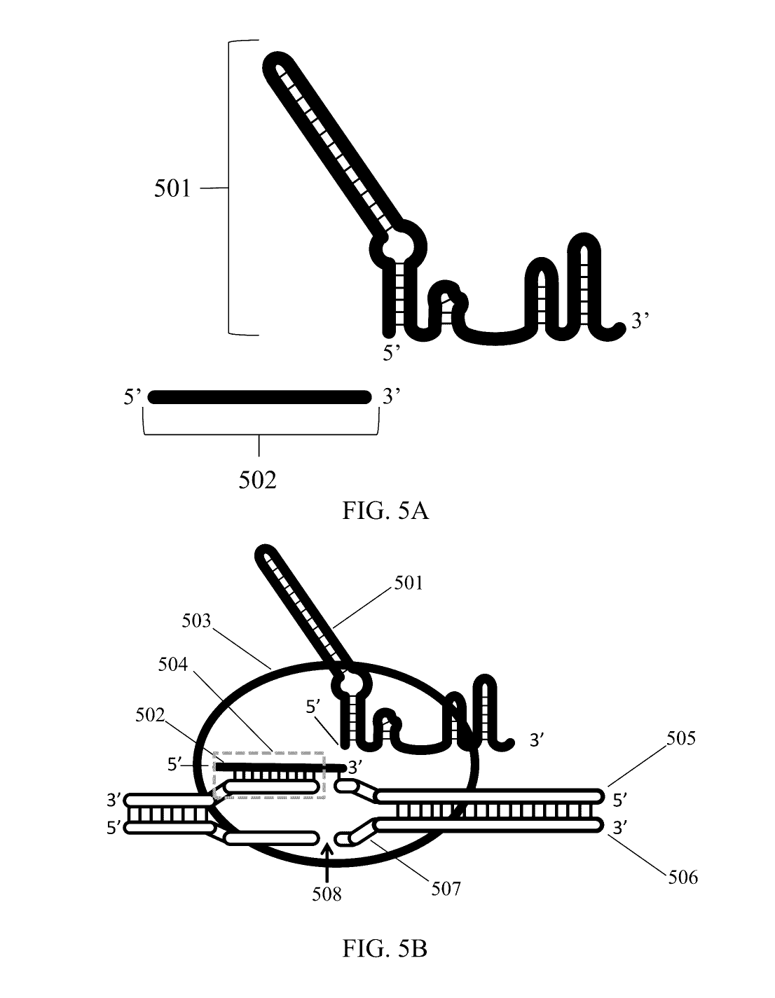

Cas9 protein was diluted to a final concentration of 200 uM in reaction buffer (20 mM HEPES, 100 mM KCl, 5 mM MgCl₂, 1 mM DTT, and 5% glycerol at pH 7.4). Each Cas polynucleotide component was added to the Cas reaction mix, wherein the final concentration of each polynucleotide was 500 nM in each reaction mix, and each reaction mix was incubated at 37° C. for 10 minutes. The Cas9 protein and the Cas polynucleotides (described in the paragraph above) form nucleoprotein complexes. FIG. 4A graphically illustrates an example of a sgRNA. FIG. 4B graphically illustrates an example of a ribonucleoprotein complex comprising Cas9/sgRNA. FIG. 5A graphically illustrates an example of a sesPN/casPN (e.g., casRNA-1/sesDNA-AAVS1 or casRNA-1/sesRNA-AAVS 1). FIG. 5B graphically illustrates an example of a nucleoprotein complex comprising a Cas9/sesPN/casPN (e.g., Cas9/casRNA-1/sesDNA-AAVS1 or Cas9/casRNA-1/sesRNA-AAVS1).

The cleavage reaction was initiated by the addition of target DNA to a final concentration of 15 nM. Samples were mixed and centrifuged briefly before being incubated for 15 minutes at 37° C. Cleavage reactions were terminated by the addition of Proteinase K (Denville Scientific, South Plainfield, N.J.) at a final concentration of 0.2 mg/mL and 0.44 mg/mL RNase A Solution (Sigma-Aldrich, St. Louis, Mo.). Samples were incubated for 25 minutes at 37° C., followed by 25 minutes at 55° C. 12 μL of the total reaction were evaluated for cleavage activity by agarose gel electrophoresis (2%, SYBR® Gold, Life Technologies, Grand Island, N.Y.).

For the AAVS-1 double-stranded DNA target, the appearance of DNA bands at ˜316 bp and ˜179 bp indicated that cleavage of the target DNA had occurred. Cleavage percentages were calculated using area under the curve values as calculated by FIJI (ImageJ; an open source Java image processing program) for each cleavage fragment and the target DNA, and dividing the sum of the cleavage fragments by the sum of both the cleavage fragments and the target DNA.

Cleavage was observed for sgRNA-AAVS and tracrRNA/crRNA-AAVS nucleoprotein particles as expected for these control nucleoprotein particles. Low cleavage percentages were observed for the casRNA-1/sesRNA-AAVS1 nucleoprotein particles.

The observed cleavage percentages of the casRNA-1/sesRNA-AAVS1 nucleoprotein complexes support that the casPN/sesPN nucleoprotein complexes as described herein facilitate Cas mediate site-specific cleavage of target double-stranded DNA.

Following the guidance of the present specification and examples, the Cas cleavage assay described in this example can be practiced by one of ordinary skill in the art with other CRISPR-Cas proteins, including, but not limited to Cas9 proteins, Cas9-like, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof, as well as their cognate polynucleotide components.

### Example 4

### Deep Sequencing Analysis for Detection of Target Modifications in Eukaryotic Cells

This example illustrates the use of deep sequencing analysis to evaluate and compare the in cell activity of selected sesPN/casPN/Cas protein nucleoprotein complexes (e.g., Cas9/casRNA-1/sesRNA-AAVS1 complexes, and Cas9/casRNA-1/sesDNA-AAVS1 complexes) relative to a selected double-stranded DNA target sequence (e.g., human AAVS-1 DNA target sequences).

A. Formation of Complexes of sesPN/casPN/Cas Protein.

A Cas protein (e.g. Streptococcus pyogenes Cas9 protein) is expressed from a bacterial expression vector in E. coli (BL21 (DE3)) and purified using affinity ion exchange and size exclusion chromatography according to methods described in Jinek, et al. (Science 337(6096):816-21(2012)). The coding sequence for the Cas protein is designed to include two nuclear localization sequences (NLS) at the C-terminus. Complexes are assembled, in triplicate at a concentration of 66 pmols Cas and 200 pmols of the casRNA-1/sesRNA-AAVS 1 or the casRNA-1/sesDNA-AAVS1. The casRNA-1/sesRNA-AAVS1 and the casRNA-1/sesDNA-AAVS1 components are mixed in equimolar amounts in an annealing buffer (1.25 mM HEPES, 0.625 mM MgCl₂, 9.375 mM KCl at pH7.5) to the desired concentration (200 pmols) in a final volume of 5 μL, incubated for 2 minutes at 95° C., removed from the thermocycler and allowed to equilibrate to room temperature. Cas protein is diluted to an appropriate concentration in binding buffer (20 mM HEPES, 100 mM KCl, 5 mM MgCl₂, 1 mM DTT, and 5% glycerol at pH 7.4) to a final volume of 5 μL and mixed with the 5 μL of heat-denatured casRNA-1/sesRNA-AAVS1 or the casRNA-1/sesDNA-AAVS 1 followed by incubation at 37° C. for 30 minutes.

B. Cell Transfections Using sesPN/casPN/Cas Protein Nucleoprotein Complexes.

casRNA-1/sesRNA-AAVS1/Cas protein and casRNA-1/sesDNA-AAVS1/Cas protein nucleoprotein complexes are transfected into K562 cells (ATCC, Manassas, Va.), using the Nucleofector® 96-well Shuttle System (Lonza, Allendale, N.J.) and the following protocol. casRNA-1/sesRNA-AAVS1/Cas protein and casRNA-1/sesDNA-AAVS1/Cas protein nucleoprotein complexes are dispensed in a 10 μL final volume into individual wells of a 96-well plate. K562 cells suspended in media are transferred from a culture flask to a 50 mL conical tube. Cells are pelleted by centrifugation for 3 minutes at 200×g, the culture medium aspirated, and the cells are washed once with calcium and magnesium-free PBS. K562 cells are then pelleted by centrifugation for 3 minutes at 200×g, the PBS aspirated and cell pellet are resuspended in 10 mL of calcium and magnesium-free PBS.

The cells are counted using the Countess® II Automated Cell Counter (Life Technologies, Grand Island, N.Y.). 2.2×10⁷ cells are transferred to a 50 ml tube and pelleted. The PBS is aspirated and the cells are resuspended in Nucleofector™ SF (Lonza, Allendale, N.J.) solution to a density of 1×10⁷ cells/mL. 20 μL of the cell suspension are added to individual wells containing 10 μL of either the casRNA-1/sesRNA-AAVS1/Cas protein or the casRNA-1/sesDNA-AAVS1/Cas protein nucleoprotein complexes and the entire volume is transferred to the wells of a 96-well Nucleocuvette™ Plate (Lonza, Allendale, N.J.). The plate is loaded onto the Nucleofector™ 96-well Shuttle™ (Lonza, Allendale, N.J.) and cells are nucleofected using the 96-FF-120 Nucleofector™ program (Lonza, Allendale, N.J.). Post-nucleofection, 70 μL Iscove's Modified Dulbecco's Media (IMDM; Life Technologies, Grand Island, N.Y.), supplemented with 10% FBS (Fisher Scientific, Pittsburgh, Pa.), penicillin and streptomycin (Life Technologies, Grand Island, N.Y.) is added to each well and 50 μL of the cell suspension are transferred to a 96-well cell culture plate containing 150 μL pre-warmed IMDM complete culture medium. The plate is then transferred to a tissue culture incubator and maintained at 37° C. in 5% CO₂ for 48 hours.

C. Target Double-Stranded DNA Generation for Deep Sequencing.

gDNA is isolated from K562 cells 48 hours after sesPN/casPN/Cas protein nucleoprotein complexes transfection using 50 μL QuickExtract DNA Extraction solution (Epicentre, Madison, Wis.) per well followed by incubation at 37° C. for 10 minutes, 65° C. for 6 minutes and 95° C. for 3 minutes to stop the reaction. The isolated gDNAs are diluted with 50 μL water and samples stored at minus 80° C.

Using the isolated gDNA, a first PCR is performed using Q5 Hot Start High-Fidelity 2× Master Mix (New England Biolabs, Ipswich, Mass.) at 1× concentration, AAVS-1 specific primers with Illumina (San Diego, Calif.) compatible adapter sequences at 0.5 μM each (SEQ ID NO. 31, SEQ ID NO. 32), 3.75 μL of gDNA in a final volume of 10 uL and amplified 98° C. for 1 minute, 35 cycles of 10 s at 98° C., 20 s at 60° C., 30 s at 72° C., and a final extension at 72° C. for 2 min. PCR reactions are diluted 1:100 in water.

A “barcoding” PCR is set up using unique primers for each sample to facilitate multiplex sequencing using manufacturer recommended index barcode sequences adapted (Illumina, San Diego, Calif.).

The barcoding PCR is performed using Q5 Hot Start High-Fidelity 2× Master Mix (New England Biolabs, Ipswich, Mass.) at 1× concentration, primers at 0.5 μM each, 1 μL of 1:100 diluted first PCR, in a final volume of 10 μL and amplified 98° C. for 1 minutes, 12 cycles of 10 s at 98° C., 20 s at 60° C., 30 s at 72° C., and a final extension at 72° C. for 2 min.

D. SPRIselect Clean-Up.

PCR reactions are pooled into a single microfuge tube for SPRIselect (Beckman Coulter, Pasadena, Calif.) bead-based clean-up of amplicons for sequencing.

To the pooled amplicons, 0.9× volumes of SPRIselect beads are added, and mixed and incubated at room temperature (RT) for 10 minutes. The microfuge tube is placed on a magnetic tube stand (Beckman Coulter, Pasadena, Calif.) until solution had cleared.

Supernatant is removed and discarded, and the residual beads are washed with 1 volume of 85% ethanol, and incubated at room temperature for 30 seconds. After incubation, ethanol is aspirated and beads are air dried at RT for 10 min. The microfuge tube is then removed from the magnetic stand and 0.25× volumes of Qiagen EB buffer (Qiagen, Venlo, Limburg) is added to the beads, mixed vigorously, and incubated for 2 minutes at room temperature. The microfuge tube is returned to the magnet, incubated until solution had cleared, and supernatant containing the purified amplicons is dispensed into a clean microfuge tube. The purified amplicon library is quantified using the Nanodrop™ 2000 system (Thermo Scientific, Wilmington, Del.) and library-quality analyzed using the Fragment Analyzer™ system (Advanced Analytical Technologies, Inc., Ames, Iowa) and the DNF-910 double-stranded DNA Reagent Kit (Advanced Analytical Technologies, Inc. Ames, Iowa).

E. Deep Sequencing Set-Up.

The amplicon library is normalized to a 4 nmolar concentration as calculated from Nanodrop values and size of the amplicons. The library is analyzed on MiSeq Sequencer (Illumina, San Diego, Calif.) with MiSeq Reagent Kit v2 (Illumina, San Diego, Calif.) for 300 cycles with two 151-cycle paired-end run plus two eight-cycle index reads.

F. Deep Sequencing Data Analysis.

The identity of products in the sequencing data are determined based on the index barcode sequences adapted onto the amplicons in the barcoding round of PCR. A computational script is used to process the MiSeq data by executing the following tasks:
Reads are aligned to the human genome (build GRCh38/38) using Bowtie (http://bowtie-bio.sourceforge.net/index.shtml) software.
Aligned reads are compared to the AAVS-1 wild-type locus sequence, reads not aligning to the AAVS-1 wild-type locus sequence part are discarded.
Reads matching wild-type locus sequence are tallied.
Reads with indels (insertion or the deletion of bases) are categorized by indel type and tallied.
Total indel reads are divided by the sum of wild-type reads and indel reads give the percent indels detected.

The in cell activity of a casRNA-1/sesRNA-AAVS1/Cas protein nucleoprotein complexes and casRNA-1/sesDNA-AAVS1/Cas protein nucleoprotein complexes through analysis of deep sequencing for detection of target modifications in eukaryotic cells provides data to demonstrate that the cas protein/casPN/sesPN constructs as described herein facilitate Cas-mediated site-specific cleavage of target double-stranded DNA in cells.

Following the guidance of the present specification and examples, in cell activity of a casPN/sesPN (e.g., casRNA, sesRNA and sesDNA) and Cas protein nucleoprotein complexes through analysis of deep sequencing described in this example can be practiced by one of ordinary skill in the art with other CRISPR-Cas proteins, including, but not limited to Cas9 proteins, Cas9-like proteins, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof, as well as their cognate polynucleotide components.

### Example 5

### Screening of Multiple sesPN Targeting Sequences

This example illustrates the use of sesPNs (e.g., sesRNAs and sesDNAs) of the present invention to evaluate and compare the modification ability of a collection of sesPNs against a selected gDNA region, for example, a human genomic DNA target in cells. Not all of the following steps are required for every screening nor must the order of the steps be as presented.

Select a DNA target region from genomic DNA.

Identify all PAM sequences (e.g. ‘NGG’) within the selected genomic region.

Identify and select one or more 20 nucleotide sequence (sesPN(s), e.g., sesRNA(s) and/or sesDNA(s)) that is/are 5′ adjacent to PAM sequences.

Selection criteria can include but is not limited to: homology to other regions in the genome, percent G-C content, melting temperature, presence of homopolymer within the spacer, and other criteria known to one skilled in the art.

Provide sesPN(s) (e.g., sesRNA(s) and/or sesDNA(s)) sequence(s) to a commercial manufacturer for synthesis.

Synthesized sesPN(s) (e.g., sesRNA(s) and/or sesDNA(s)) is/are used as described in the Experimental section herein with a cognate casPN (e.g., casRNA or casDNA) and a cognate Cas protein (e.g., a Cas9 protein).

In vitro cleavage percentages and specificity associated with each sesPN(s) (e.g., sesRNA(s) and/or sesDNA(s)) are compared following the guidance of Example 3, Cas Cleavage Assays.
(a) A single sesPN (e.g., sesRNA or sesDNA): If only a single sesPN is identified or selected, its cleavage percentage and specificity for the DNA target region is determined. If so desired, cleavage percentage and/or specificity are altered using methods of the present invention including use of affinity sequences, cross-linking, and/or ligands.
(b) Multiple sesPNs (e.g., sesRNAs and/or sesDNAs): The percentage cleavage data and site specificity data obtained from the cleavage assays are compared between different sesPN to identify the sesPN having the best cleavage percentage and highest specificity. Cleavage percentage data and specificity data provide criteria on which to base choices for a variety of applications. For example, in some situations the specificity of the cleavage site may be relatively more important than the cleavage percentage. If so desired, cleavage percentage and/or specificity are altered using methods of the present invention including use of affinity sequences, cross-linking, and/or ligands.

Optionally or instead of the in vitro analysis, in cell percent indels detected and specificity are compared following the guidance of Example 4, Deep Sequencing Analysis for Detection of Target Modifications in Eukaryotic Cells.
(a) A single sesPN (e.g., sesRNA or sesDNA): If only a single sesPN is identified, its percent indels detected and specificity for the DNA target region is determined. If so desired, percent indels detected and/or specificity are altered using methods of the present invention including use of affinity sequences, cross-linking, and/or ligands.
(b) Multiple sesPN(s) (e.g., sesRNA(s) and/or sesDNA(s)): The percentage indels detected data and site specificity data obtained from the cleavage assays are compared between different sesPNs to identify the sesPN having the best percent indels detected and highest specificity. Percent indels detected data and specificity data provide criteria on which to base choices for a variety of applications. For example, in some situations the specificity of the cleavage site may be relatively more important than the percent indels detected. If so desired, percent indels detected and/or specificity are altered using methods of the present invention including use of affinity sequences, cross-linking, and/or ligands.

Following the guidance of the present specification and examples, the screening described in this example can be practiced by one of ordinary skill in the art with other CRISPR-Cas proteins, including, but not limited to Cas9 proteins, Cas9-like, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof, as well as their cognate polynucleotide components.

### Example 6

### Identification and Screening of CRISPR RNA and Trans-Activating CRISPR RNA

This example illustrates the method through which CRISPR RNAs (crRNAs) and trans-activating CRISPR RNAs (tracrRNAs) of a Class 2 CRISPR-Cas system can be identified. The method presented here is adapted from Chylinski, et al., (RNA Biol; 10(5):726-37 (2013)). Not all of the following steps are required for screening nor must the order of the steps be as presented. The following method is described with reference to Class 2 Type II CRISPR-Cas Systems but the method is readily modifiable by one of ordinary skill in the art to be applied to other Class 2 CRISPR-Cas systems as well, for example, Class 2 Type V CRISPR-Cas systems.

A. Identify a Bacterial Species Containing a Type II CRISPR-Cas System.

Using the Basic Local Alignment Search Tool (BLAST, blast.ncbi.nlm.nih.gov/Blast.cgi), a search of various species' genomes is conducted to identify Cas or Cas-like proteins. Type II CRISPR-Cas systems exhibit a high diversity in sequence across bacterial species, however Cas orthologs exhibit conserved domain architecture of central HNH endonuclease domain and a split RuvC/RNase H domain. Primary BLAST results are filtered for identified domains; incomplete or truncated sequences are discarded and Cas9 orthologs identified.

When a Cas ortholog is identified in a species, sequences adjacent to the Cas ortholog's coding sequence are probed for other Cas proteins and an associated repeat-spacer array in order to identify all sequences belonging to the CRISPR-Cas locus. This may be done by alignment to other Type II CRISPR-Cas loci already known in the public domain, with the knowledge that closely related species exhibit similar CRISPR-Cas locus architecture (i.e., Cas protein composition, size, orientation, location of array, location of tracrRNA, etc.).

B. Identification of Putative crRNA and tracrRNA.

Within the locus, the crRNAs are readily identifiable by the nature of their repeat sequences interspaced by fragments of foreign DNA and make up the repeat-spacer array. If the repeat sequence is known for a species, it is identified in and retrieved from the CRISPRdb database (crispr.u-psud.fr/crispr/). If the repeat sequence is not known to be associated with a species, repeat sequences are predicted using CRISPRfinder software (crispr.u-psud.fr/Server/) using the sequence identified as a Type II CRISPR-Cas locus for the species as described above.

Once the sequence of the repeat sequence is identified for the species, the tracrRNA is identified by its sequence complementarity to the repeat sequence in the repeat-spacer array (tracr anti-repeat sequence). In silico predictive screening is used to extract the anti-repeat sequence to identify the associated tracrRNA. Putative anti-repeats are screened, for example, as follows.

The identified repeat sequence for a given species is used to probe the CRISPR-Cas locus for the anti-repeat sequence (e.g., using the BLASTp algorithm or the like). The search is typically restricted to intronic regions of the CRISPR-Cas9 locus.

An identified anti-repeat region is validated for complementarity to the identified repeat sequence.

A putative anti-repeat region is probed both 5′ and 3′ of the putative anti-repeat for a Rho-independent transcriptional terminator (TransTerm HP, transterm.cbcb.umd.edu/).

Thus, the identified sequence comprising the anti-repeat element and the Rho-independent transcriptional terminator is determined to be the putative tracrRNA of the given species.

C. Preparation of RNA-Seq Library.

The putative crRNA and tracrRNA that were identified in silico are further validated using RNA sequencing (RNAseq).

Cells from species from which the putative crRNA and tracrRNA were identified are procured from a commercial repository (e.g., ATCC, Manassas, Va.; DSMZ, Braunschweig, Germany).

Cells are grown to mid-log phase and total RNA prepped using Trizol reagent (Sigma-Aldrich, St. Louis, Mo.) and treated with DNaseI (Fermentas, Vilnius, Lithuania).

10 ug of the total RNA is treated with Ribo-Zero rRNA Removal Kit (Illumina, San Diego, Calif.) and the remaining RNA purified using RNA Clean and Concentrators (Zymo Research, Irvine, Calif.).

A library is then prepared using TruSeq Small RNA Library Preparation Kit (Illumina, San Diego, Calif.) following the manufacturer's instructions, which results in the presence of adapter sequences associated with the cDNA.

The resulting cDNA library is sequenced using MiSeq Sequencer (Illumina, San Diego, Calif.).

D. Processing of Sequencing Data.

Sequencing reads of the cDNA library can be processed using the following method.

Adapter sequences are removed using cutadapt 1.1 (pypi.python.org/pypi/cutadapt/1.1) and 15 nt are trimmed from the 3′ end of the read to improve read quality.

Reads are aligned back to each respective species' genome (from which the putative tracrRNA was identified) with a mismatch allowance of 2 nucleotides.

Read coverage is calculated using BedTools (bedtools.readthedocs.org/en/latest/).

Integrative Genomics Viewer (IGV, www.broadinstitute.org/igv/) is used to map the starting (5′) and ending (3′) position of reads. Total reads retrieved for the putative tracrRNA are calculated from the SAM file of alignments.

The RNA-seq data is used to validate that a putative crRNA and tracrRNA element is actively transcribed in vivo. Confirmed hits from the composite of the in silico and RNA-seq screens are validated for functional ability of the identified crRNA and tracrRNA sequences to support Cas mediated cleavage of a double-stranded DNA target using methods outline herein (see Examples 1, 2, and 3).

Following the guidance of the present specification and the examples herein, the identification of novel crRNA and tracrRNA sequences can be practiced by one of ordinary skill in the art.

E. Design of casPN and sesPN.

The design of sesPNs is detailed in Example 5. Additional modification to the 5′ and/or 3′ of the sesPN are evaluated using methods described in Example 3 and 4.

The casPN is designed using an identified crRNA for a given species (e.g., Streptococcus pyogenes crRNA). The target nucleic acid binding sequence of a crRNA is removed, and the retained repeat sequence of the crRNA is used in combination with the species' tracrRNA to form a casPN (here, e.g., a casRNA). A distinct sesPN is used to direct Cas protein targeting. An illustration of such a sesPN and a casPN is represented in FIG. 1E and FIG. 1F.

Alternatively, the target nucleic acid binding sequence of a crRNA is removed, and the retained repeat sequence of the crRNA is used in combination with the species' tracrRNA to form a casPN (here, e.g., a casRNA), wherein the retained repeat sequence of the crRNA and the species' tracrRNA are covalently linked using a nucleotide loop sequence (e.g., a tetraloop). The retained repeat sequence of the crRNA is joined to the tracrRNA anti-repeat sequence as described in Jinek, et al., (Science 337(6096):816-21 (2012)) and Ran, F A. et al. (“In vivo genome editing using Staphylococcus aureus Cas9,” Nature, 520(7546):186-91 (2015)). An example of such a casPN and an accompanying sesPN is represented in FIG. 2C and FIG. 2D.

Following the guidance of the present specification and examples, the identification of crRNA and tracrRNA and the subsequent design of sesPN and casPN as described in this example can be practiced by one of ordinary skill in the art for other CRISPR-Cas proteins and their cognate polynucleotide components, including, but not limited to Cas9 proteins, Cas9-like proteins, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof.

### Example 7

### Cross-Linking of Cas Protein and sesPNs

This example describes the modification of sesPNs of the present invention to include a cross-linking agent, as well as modification of selected amino acid residues in the Class 2 Type II CRISPR-Cas protein. This combination of a modified Cas protein and modified sesPN illustrates another mechanism that can be used to bring the sesPN into proximity with the RNA/DNA binding channel of the Cas protein.

The two cysteine (Cys, C) residues present in wild-type SpyCas9 (Streptococcus pyogenes serotype M1, UniProtKB-Q99ZW2 (CAS9_STRP1), GenBank: AAK33936.1: SEQ ID NO. 20) were mutated to serine residues (Ser, S) (C80S, C574S). Single Cys point mutations were then introduced as described in Spanggord, R J, and Beal, P A, “Site-specific modification and RNA cross-linking of the RNA-binding domain of PKR” Nucleic Acids Res 28: 1899-1905 (2000).

Briefly, the nucleic acid coding sequence of SpyCas9 was produced with a substitution of a codon coding for cysteine (TGC) for the original wild-type codon to create the desired introduction of cysteine at discrete positions along the RNA/DNA binding channel of the encoded Cas protein. The Cas9 nucleic acid sequence (e.g., RNA/DNA) binding channel is described in Jiang, et al., “Structures of a CRISPR-Cas9 R-loop complex primed for DNA cleavage,” Science. February 19; 351(6275):867-71 (2016) and Nishimasu, H., et al., “Crystal structure of Cas9 in complex with guide RNA and target DNA,” Cell February 27; 156(5):935-49 (2014).

The amino acid position corresponding to the introduction of Cys codon was designed to be an optimal distance to the thiol of the thiolated sesRNA for S—S cross-linking. Distances where chosen according to the predicted length of the carbon chain linkages in the thiol moiety used in the sesRNA (example lengths for C3 and C6 linkages range between 7 and 10 Å as discussed in Green, N. S., et al., “Quantitative evaluation of the lengths of homobifunctional protein cross-linking reagents used as molecular probes,” Prot. Sci., 10:1293-1304 (2001)). The resulting Cas9-Cys protein variants are presented in Table 2. The SpyCas9-Cys protein was then expressed and purified as described in Jinek, et al., (Science 337(6096):816-21 (2012)) and concentrated to 1 mg/ml.

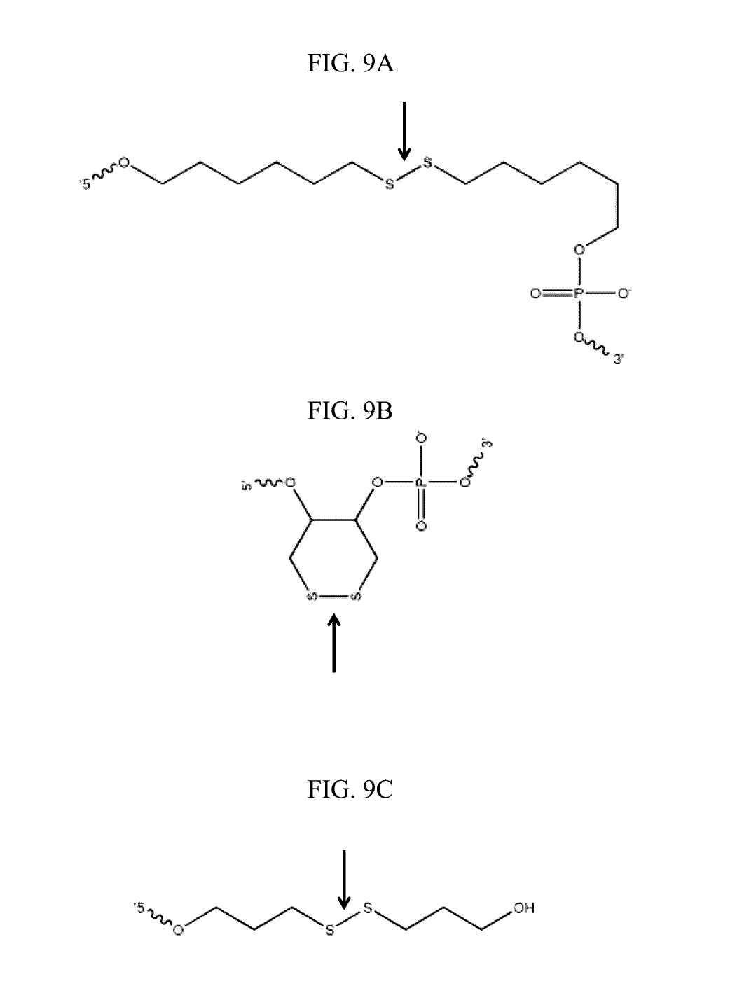

The sesRNA sequence (RNA-A; SEQ ID NO. 2) was selected to target the AAVS-1 DNA sequence. Thiol functionalities were designed along the length of the sesRNA sequence at positions predicted to be at an accessible distance (preferably an optimal distance) to promote S—S formation between the sesRNA and the Cys residue of the modified Cas9-Cys protein variants. Exemplary thiol functionalities are shown in FIG. 9A (Thiol C6), FIG. 9B (Dithiol Phosphoramidite, DTPA), and FIG. 9C (Thiol C3). The thiol positions for each of the thiolated sesRNAs and the Cas9-Cys protein variant tested with each thiolated sesRNAs are presented in Table 2.

TABLE 2
Design for Cas9-Cys Protein Variant/Thiolated
sesRNA Biochemical Cleavage Reactions

| | Thiol | | | | | | | |
|---|---|---|---|---|---|---|---|---|
| sesRNA | position | Cas9-Cys variants | | | | | | |
| RNA-A | none-WT | | | | | | | |
| RNA-B | 1[ThiolC6] | V922C | T924C | E1007C | F1008C | V1009C | Y1010C | |
| RNA-C | 5[DTPA] | K510C | R586C | N588C | | | | |
| RNA-D | 6[DTPA] | K510C | R586C | N588C | | | | |
| RNA-E | 8[DTPA] | K890C | T893C | Q894C | R895C | | | |
| RNA-F | 9[DTPA] | K890C | T893C | Q894C | R895C | | | |
| RNA-G | 10[DTPA] | E779C | | | | | | |
| RNA-H | 13[DTPA] | R494C | M495C | | | | | |
| RNA-I | 14[DTPA] | R494C | M495C | | | | | |
| RNA-J | 15[DTPA] | Y450C | I448C | | | | | |
| RNA-K | 16[DTPA] | R447C | I448C | | | | | |
| RNA-L | 17[DTPA] | R447C | I448C | | | | | |
| RNA-M | 19[DTPA] | Y72C | R403C | T404C | F405C | D406C | N407C | F164C |
| RNA-N | 20[ThiolC3] | Y72C | R403C | T404C | F405C | D406C | N407C | F164C |

For biochemical cleavage Cas9-Cys proteins and thiolated sesRNAs were each reduced with 100× molar excess of Tris (2-carboxyethyl) phosphine (TCEP) reagent at room temperature for 2 hours in reaction buffer (20 mM HEPES, 100 mM KCl, 5 mM MgCl₂, and 5% glycerol at pH 7.4) following the manufacturer's protocol (Integrated DNA Technologies (Coralville, Iowa)). To cross-link, the reduced Cas9-Cys proteins and the reduced thiolated sesRNAs (or control sesRNA RNA-A) were incubated together at room temperature for 2 hours in the reaction buffer. The casRNA-2 sequence was as follows: 5′-GUUUUAGAGC UAGAAAUAGC AAGUUAAAAU AAGGCUAGUC CGUUAUCAAC UUGAAAAAGU GGCACCGAGU CGGUGCU-3′ (SEQ ID NO. 21). The sequence of the casRNA-2 was provided to a commercial manufacturer for synthesis.

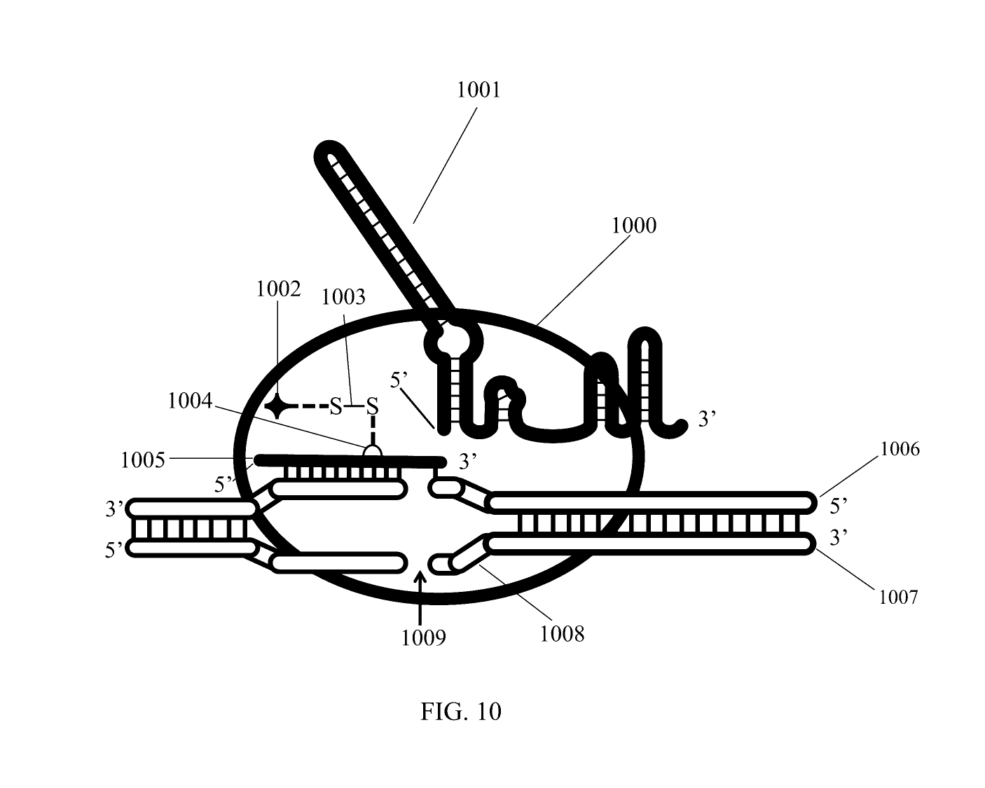

The casRNA-2 was then added to the Cas9-Cys/sesRNA adduct to form the Cas9-Cys/thiolated sesRNA/casRNA-2 ribonucleoprotein (RNP) complex. An example of such a ribonucleoprotein complex is graphically illustrated in FIG. 10. The biochemical cleavage reaction was performed as described in Example 3, but without added DTT. The cleavage reactions were evaluated for cleavage activity by agarose gel electrophoresis and cleavage percentages calculated as described in Example 3.

The results of the Cas cleavage assays using the AAVS-1 target double-stranded DNA (Example 2) and the Cas9-Cys/thiolated sesRNA/casRNA-2 RNP complexes are summarized in Table 3.

TABLE 3
RNA Design and Results of Biochemical Cleavage Reaction for Cas9-casRNA/thiolated
sesRNA

| | sesRNA Thiol Sites | |
|---|---|---|
| Designation for | (locations in the sesRNA sequence are | Biochemical |
| thiolated sesRNA | numbered 5′ to 3′) | cleavage |
| RNA-A | GGGGCCACUAGGGACAGGAU | BloD* |
| | (SEQ ID NO. 22) | |
| RNA-B | ThiolC6 substituted for G in position 1 of | ++ |
| | SEQ ID NO. 22 | |
| RNA-C | DTPA substituted for C in position 5 of | + |
| | SEQ ID NO. 22 | |
| RNA-D | DTPA substituted for C in position 6 of | BloD |
| | SEQ ID NO. 22 | |
| RNA-E | DTPA substituted for C in position 8 of | BloD |
| | SEQ ID NO. 22 | |
| RNA-F | DTPA substituted for U in position 9 of | ++ |
| | SEQ ID NO. 22 | |
| RNA-G | DTPA substituted for A in position 10 of | + |
| | SEQ ID NO. 22 | |
| RNA-H | DTPA substituted for G in position 13 of | + |
| | SEQ ID NO. 22 | |
| RNA-I | DTPA substituted for A in position 14 of | + |
| | SEQ ID NO. 22 | |
| RNA-J | DTPA substituted for C in position 15 of | BloD |
| | SEQ ID NO. 22 | |
| RNA-K | DTPA substituted for A in position 16 of | BloD |
| | SEQ ID NO. 22 | |
| RNA-L | DTPA substituted for G in position 17 of | BloD |
| | SEQ ID NO. 22 | |
| RNA-M | DTPA substituted for A in position 19 of | ++ |
| | SEQ ID NO. 22 | |
| RNA-N | Thio1C3 3′ modification to SEQ ID NO. 22 | ++ |

*BloD = Below Limit of Detection

The biochemical cleavage data for the Cas9-Cys/thiolated sesRNA/casRNA-2 RNP complexes demonstrate that the Cas9-Cys/thiolated sesRNA/casRNA constructs as described herein facilitate Cas mediated site-specific cleavage of target double-stranded DNA.

Following the guidance of the present specification and examples, the Cas cleavage assay described in this example can be practiced by one of ordinary skill in the art with other CRISPR-Cas protein variants (e.g., Cas-Cys variants), including, but not limited to variants of Cas9 proteins, Cas9-like, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and modifications thereof, as well as their cognate polynucleotide components.

### Example 8

### Cas9-dCsy4 Fusion Protein and sesRNA with the dCsy4 Binding Domain

This example describes the use of a Cas9 fusion with the RNA binding protein dCsy4 (an enzymatically inactive variant of the Pseudomonas aeruginosa Csy4 (strain UCBPP-PA14)) and a sesPN modified to include the RNA binding sequence corresponding to the dCsy4 at the 5′ end of the sesPN. This combination of a Cas9 fusion to an RNA binding protein and attachment of the corresponding RNA binding protein binding sequence to an sesPN illustrates another mechanism that can be used to bring the sesPN into proximity with the RNA/DNA binding channel of the Cas protein.

Cas9 was fused at its N-terminal end with the C-terminal end of the dCsy4 RNA binding domain or Cas9 was fused at its C-terminal end with the N-terminal end of the dCsy4 RNA binding domain (dCsy4-Cas9 and Cas9-dCsy4, respectively, herein referred to together as (dCsy4)Cas9. The sesRNA was designed to include a Csy4 hairpin RNA (i.e., the Csy4 binding sequence) at the 5′ end. The Csy4 hairpin was connected with RNA linkers of various lengths (10-40 bases) to sesRNAs to produce Csy4-sesRNAs. Csy4-sesRNAs were produced as described in Example 1.

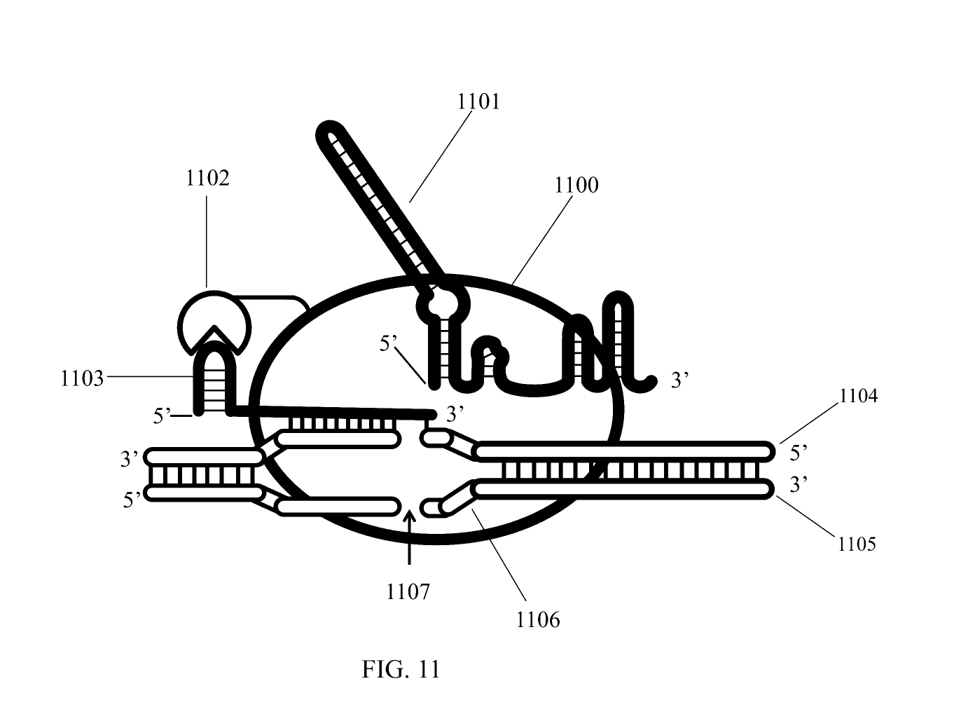

For the biochemical cleavage reaction the (dCsy4)Cas9 fusion proteins were each incubated with a Csy4-sesRNA. The resulting (dCsy4)Cas9/Csy4-sesRNA complexes were incubated with a casRNA-2 to form the (dCsy4)Cas9/Csy4-sesRNA/casRNA-2 RNP complex. An example of such a ribonucleoprotein complex is graphically illustrated in FIG. 11. The biochemical cleavage reaction was performed as previously described (Example 3). The results of the biochemical cleavage assays are presented in Table 4. Use of either dCsy4-Cas9 fusion protein or the Cas9-dCsy4 fusion protein provided similar results.

TABLE 4
RNA Design and Results of Biochemical Cleavage Reaction for
(dCsy4)Cas9-casRNA/Csy4-sesRNA

| Csy4- | Csy4 Hairpin | LINKER | AAVS-1 Spacer | |
|---|---|---|---|---|
| sesRNA | Sequence | Sequence | Sequence | Cleavage |
| Csy4- | GGAGAGUUCAC | CUAAGAAUGCU | GGGGCCACU | ++ |
| sesRNA-40 | UGCCGUAUAGG | CUUCCGAUCUG | AGGGACAGG | |
| | CAG (SEQ ID NO. | CUACUCUAAGC | AU (SEQ ID | |
| | 23) | AUAUCGU (SEQ | NO. 2) | |
| | | ID NO. 24) | | |
| Csy4- | SEQ ID NO. 23 | UGCUCUUCCGA | SEQ ID NO. 2 | + |
| sesRNA-30 | | UCUGCUACUCU | | |
| | | AAGCAUAU | | |
| | | (SEQ ID NO. 25) | | |
| Csy4- | SEQ ID NO. 23 | UGCUCUUCCGA | SEQ ID NO. 2 | BLoD* |
| sesRNA-20 | | UCUGCUACU | | |
| | | (SEQ ID NO. 26) | | |
| Csy4- | SEQ ID NO. 23 | AUCUGCUACU | SEQ ID NO. 2 | BLoD |
| sesRNA-10 | | (SEQ ID NO. 27) | | |

*BloD = Below Limit of Detection

These data demonstrate that the (dCsy4)Cas9/Csy4-sesRNA/casRNA RNP complex constructs as described herein facilitate Cas protein mediated site-specific cleavage of target double-stranded DNA.

Following the guidance of the present specification and examples, the Cas cleavage assay described in this example can be practiced by one of ordinary skill in the art using other CRISPR-Cas protein variants (e.g., (dCsy4)Cas variants), including, but not limited to variants comprising Cas9 proteins, Cas9-like proteins, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and modifications thereof, as well as their cognate polynucleotide components.

### Example 9

### Crosslinking of Cpf1 Protein Variants and Thiolated sesPNs

This example describes the modification of sesPNs of the present invention to include a cross-linking agent, as well as modification of selected amino acid residues in the CRISPR-Cas Class 2 Type V CRISPR Cpf1 protein. This combination of a modified Cas protein and modified sesPN provides another example of using cross-linking to bring the sesPN into proximity with the RNA/DNA binding channel of the Cas protein.

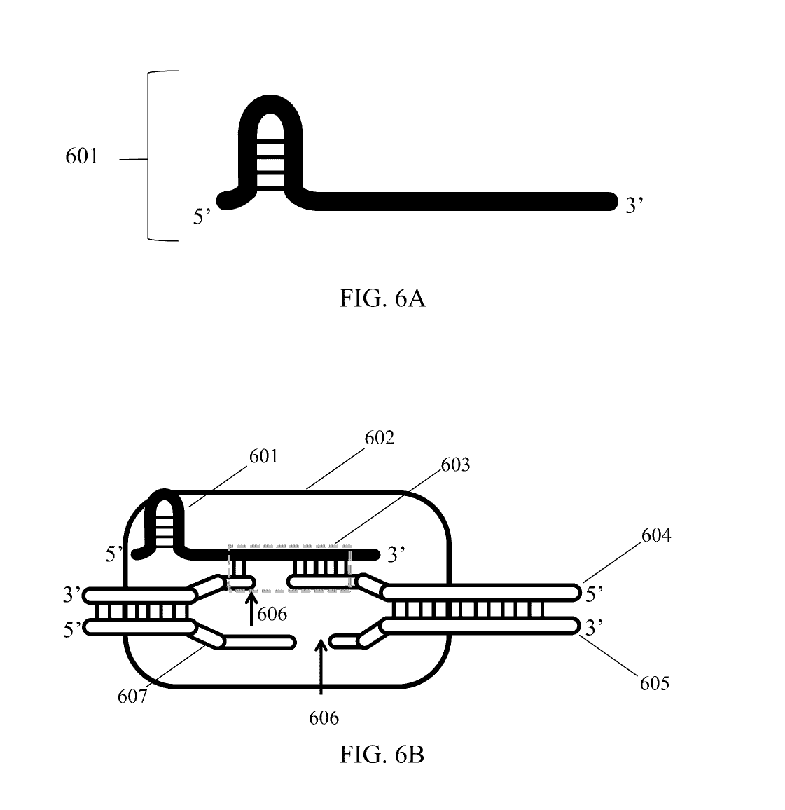

An example of a wild-type Cpf1 crRNA is graphically illustrated in FIG. 6A. An example of a wild-type Cpf1/crRNA ribonucleoprotein complex is graphically illustrated in FIG. 6B.

The twelve wild-type Cys residues in Acidaminococcus spp. Cpf1 (A. spp. Cpf1; UniProtKB-U2UMQ6 (CPF1_ACISB); SEQ ID NO. 33) protein are mutated to Ser. Single Cys point mutations are introduced into the modified Acidaminococcus spp. Cpf1 at discrete positions along the RNA/DNA binding channel to yield Cpf1-Cys protein variants. The Cys residues are designed to be in optimal distance to the thiolated sesRNA for S—S cross-linking as discussed above. Thiol functionalities are designed along the sesRNA sequence in positions predicted to be in optimal distance to promote S—S formation between the thiolated sesRNA and Cpf1-Cys protein variants. Cpf1-Cys protein variants are purified and concentrated.

Proposed modification sites for Cpf1-Cys protein variants and thiolated sesRNA are presented in Table 5. Numbering of the Cpf1-Cys protein is based on the numbering of the Cpf1 protein as presented by Yamano T, et al. (“Crystal Structure of Cpf1 in Complex with Guide RNA and Target DNA,” Cell 165(4):949-62 (2016)). Numbering of the sesRNA positions is relative to the AAVS1 spacer of the sesRNA.

TABLE 5
Design for Cpf1-Cys Protein Variant/Thiolated sesRNA Biochemical Cleavage Reactions

| | sesRNA Thiol Sites | Exemplary AAVS1 sesRNA spacer |
|---|---|---|
| Residue Number in | Corresponding to the | into which the thiol modifications are |
| A. sp Cpf1 Protein | Cpf1-Cys Protein | introduced at the locations indicated |
| for Cys Modification | Variant (col. 1) | in col. 2 |
| Arg18 | 1 [ThiolC6] | 5′UCUGUCCCCUCCACCCCACA3′ |
| | | (SEQ ID NO. 28) |
| Ser14 | 1[ThiolC6] | SEQ ID NO. 28 |
| Lys15 | 1[ThiolC6] | SEQ ID NO. 28 |
| Asp1021 | 1[ThiolC6] | SEQ ID NO. 28 |
| Arg18 | 2[DTPA] | SEQ ID NO. 28 |
| His872 | 2[DTPA] | SEQ ID NO. 28 |
| Phe788 | 2[DTPA] | SEQ ID NO. 28 |
| Lys530 | 2[DTPA] | SEQ ID NO. 28 |
| Lys48 | 3[DTPA] | SEQ ID NO. 28 |
| Tyr47 | 4[DTPA] | SEQ ID NO. 28 |
| Lys51 | 4[DTPA] | SEQ ID NO. 28 |
| Leu310 | 6[DTPA] | SEQ ID NO. 28 |
| Lys307 | 7[DTPA] | SEQ ID NO. 28 |
| Arg192 | 7[DTPA] | SEQ ID NO. 28 |
| Phe306 | 7[DTPA] | SEQ ID NO. 28 |
| Lys200 | 7[DTPA] | SEQ ID NO. 28 |
| Gln410 | 21[ThiolC3] | 5′UCUGUCCCCUCCACCCCACAG3′ |
| | | (SEQ ID NO. 29) |
| His293 | 21[ThiolC3] | SEQ ID NO. 29 |
| Asn288 | 21[ThiolC3] | SEQ ID NO. 29 |
| Gln410 | 21[ThiolC3] | SEQ ID NO. 29 |
| His368 | 18[DTPA] | SEQ ID NO. 28 |
| Lys370 | 15[DTPA] or | SEQ ID NO. 28 |
| | 16[DTPA] | |
| Glu272 | 17[DTPA] | SEQ ID NO. 28 |
| Arg301 | 10[DTPA] | SEQ ID NO. 28 |
| Val952 | 12[DTPA] | SEQ ID NO. 28 |

For biochemical cleavage reactions Cpf1-Cys proteins and thiolated sesRNAs are each reduced with 100× molar excess of TCEP reagent at room temperature for 2 hours in reaction buffer without dithiothreitol (DTT) following the manufacturer's protocol, (Integrated DNA Technologies, Coralville, Iowa). To cross-link, the reduced Cpf1-Cys proteins and the reduced thiolated sesRNAs are incubated together at room temperature for 2 hours in the reaction buffer.

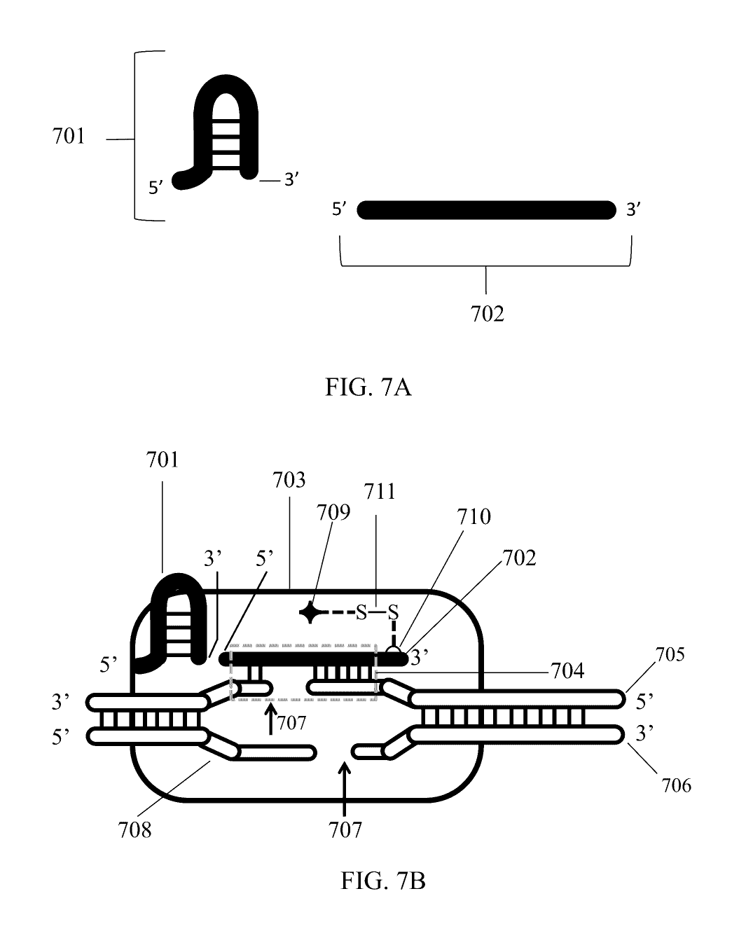

The casRNA-3 is then added to the Cpf1-Cys/thiolated sesRNA adduct to form the Cpf1-Cys/thiolated sesRNA/casRNA-3 RNP complexes. The sequence of the casRNA-3 was provided to a commercial manufacturer for synthesis. The casRNA-3 sequence is as follows 5′-AAUUUCUACU CUUGUAGAU-3′ (SEQ ID NO. 30). An example of a Cpf1 casRNA-3/sesRNA is illustrated in FIG. 7A. An example of a Cpf1-Cys/thiolated sesRNA/casRNA-3 ribonucleoprotein complex is graphically illustrated in FIG. 7B. The biochemical cleavage reactions are performed essentially as described in Example 3, but without added DTT.

The resulting data is used to demonstrate that the Cpf1-Cys/thiolated sesRNA/casRNA RNP complex constructs as described herein facilitate Cas protein mediate site-specific cleavage of target double-stranded DNA.

Following the guidance of the present specification and examples, the Cas protein modifications, sesPN modifications, and Cas cleavage assays described in this example can be practiced by one of ordinary skill in the art other CRISPR-Cas protein variants (e.g., Cpf1-Cys variants), including, but not limited to variants of Cas9 proteins, Cas9-like proteins, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and modifications thereof, as well as their cognate polynucleotide components.

### Example 10

### Crosslinking of Cpf1 Protein Variants with Thiolated sesPNs and UV-Cross-Linkable casPN

This example describes the modification of the sesPN and the casPN of a CRISPR-Cas Class 2 Type V Cas protein (e.g., Acidaminococcus spp. Cpf1) to allow for cross-linking of both the sesPN and casPN to a Cas protein with independent, orthogonal chemistry cross-linking (e.g., thiolation and UV cross-linking chemistry). This combination of a modified Cas protein, modified sesPN, and modified casPN (i.e., Cpf1 pseudoknot) provides an example of using cross-linking to bring the sesPN into proximity with the RNA/DNA binding channel of the Cas protein and to enhance the association of the casPN with the Cas protein.

As described in Example 9, the twelve wild-type Cys residues in Acidaminococcus spp. Cpf1 are mutated to Ser. Amino acid residues in the Acidaminococcus spp. Cpf1 and nucleotide positions in a sesRNA are evaluated to predicted protein amino acids that are in optimal distance to nucleotide positions in the sesRNA to promote S—S formation between a thiolated sesRNA and a Cpf1-Cys. Single Cys point mutations are introduced in Acidaminococcus spp. Cpf1 at discrete positions along the RNA/DNA binding channel that are determined to be in optimal distance to thiolated residues in sesRNA to facilitate S—S cross-linking. Thiol functionalities are similarly designed along the sesRNA sequence in positions predicted to provide optimal distance to promote S—S formation between a thiolated sesRNA and a Cpf1-Cys.

Additionally, cross-linking moieties for UV cross-linking are introduced in the casRNA-3 to provide a modified UV-casRNA-3. Cpf1-Cys proteins are purified and concentrated. A combination of thiolated sesRNA cross-linked to Cpf1-Cys with thiol chemistry and the casRNA-3 cross-linked to Cpf1-Cys with a UV cross-linking chemistry are used in Cas biochemical cleavage reactions (UV-casRNA-3/Cpf1-Cys/thiolated sesRNA. Exemplary positions for introduction of UV cross-linking moieties on the casRNA-3 are shown in Table 6. Numbering of the modified casRNA-3 is based on the numbering of the Cpf1 crRNA as presented by Yamano, T., et al. (“Crystal Structure of Cpf1 in Complex with Guide RNA and Target DNA,” Cell 165(4):949-62 (2016)).

TABLE 6
Modified casRNA-3/UV cross-linker sites
Base: −19 (5’end)
Base: −12
Base: −1
Base: −2
Base: −15

For biochemical cleavage Cpf1-Cys proteins and thiolated sesRNAs are each reduced with 100× molar excess of TCEP reagent at room temperature for 2 hours in reaction buffer without dithiothreitol (DTT) following the manufacturer's protocol (Integrated DNA Technologies, Coralville, Iowa). To cross-link, the reduced Cpf1-Cys proteins and the reduced thiolated sesRNAs are incubated together at room temperature for 2 hours in the reaction buffer. The modified casRNA-3 is cross-linked to the Cpf1-Cys/thiolated sesPN using UV light (after the method of Chodosh L A, “UV cross-linking of proteins to nucleic acids,” Curr Protoc Mol Biol. 2001 May; Chapter 12:Unit 12.5) to form the UV-casRNA-3/Cpf1-Cys/thiolated sesRNA RNP complex. The biochemical cleavage reactions are performed as described in Example 3, but without DTT added.

The resulting data is used to demonstrate that the UV-casPN-3/Cpf1-Cys/thiolated sesPN RNP complex constructs as described herein facilitate Cas protein mediated site-specific cleavage of target double-stranded DNA.

Alternatively, the thiol and UV cross-linking chemistry can be switched between the sesPN and casPN (UV-sesPN and thiolated casPN). The Acidaminococcus spp. Cpf1 is modified as described in Example 9 and Cys residues are introduced into the protein at positions to provide optimal distance to promote S—S formation between a thiolated casPN and a Cpf1-Cys. Examples of residues for Cpf1-Cys modification for S—S cross-linking with the casPN are shown in Table 7.

TABLE 7
Residue number in
Acidaminococcus spp. Cpf1
Met806
Lys943
Met1018
Phe864

Following the guidance of the present specification and examples, the Cas protein modifications, sesPN modifications, and Cas cleavage assays described in this example can be practiced by one of ordinary skill in the art other CRISPR-Cas protein variants (e.g., Cpf1-Cys variants), including, but not limited to variants of Cas9 proteins, Cas9-like proteins, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and modifications thereof, as well as their cognate polynucleotide components

### Example 11

### dCsy4-Cpf1 Fusion and sesRNA with Csy4-Hairpin

This example describes the use of a Cpf1 (e.g., Acidaminococcus spp. Cpf1) fusion with the RNA binding domain of dCsy4 protein (an enzymatically inactive variant of the Pseudomonas aeruginosa (UCBPP-PA14)) and a sesRNA modified to include the RNA binding sequence corresponding to the dCsy4. This combination of a Cpf1 fusion to an RNA binding protein binding domain and attachment of the corresponding RNA binding protein binding sequence to an sesRNA further illustrates a mechanism that can be used to bring the sesPN into proximity with the RNA/DNA binding channel of the Cas protein.

A. sesRNA with Csy4 Hairpin.

The C-terminal end of the Acidaminococcus spp. Cpf1 is fused to the N- or C-terminal end of a dCsy4 RNA binding domain, or the dCsy4 is fused to a site internal to the Cpf1 protein (referred to collectively as (dCsy4)Cpf1). Examples of insertion sites in Cpf1 to insert the dCsy4 RNA binding domain to create (dCsy4)Cpf1 fusion proteins are presented in Table 8. Linker sequences were used before and after the inserted dCsy4.

TABLE 8
Residue number in Acidaminococcus
spp. Cpf1
N1090
T402
D1208
R1194
E441
C-terminus
N-terminus

sesRNA is designed to include a Csy4 hairpin RNA at the 3′ end (Csy4-sesRNA). The Csy4 hairpin is connected to the sesRNA using RNA linkers of various lengths (e.g., 10-40 bases).

For the biochemical cleavage reaction the (dCsy4)Cpf1 fusion proteins are each incubated with a Csy4-sesRNA. The resulting (dCsy4)Cpf1/Csy4-sesRNA complexes are incubated with a casRNA-3 to form the (dCsy4)Cpf1/Csy4-sesRNA/casRNA-3 RNP complex. The biochemical cleavage reaction is performed as previously described (Example 3).

These data are used to demonstrate that the (dCsy4)Cpf1/Csy4-sesPN/casPN nucleoprotein complex constructs as described herein facilitate Cas protein mediate site-specific cleavage of target double-stranded DNA.

B. sesPN with First Csy4 Hairpin and casPN with Second Csy4 Hairpin.

This example describes the use of a Cpf1 (e.g., Acidaminococcus spp. Cpf1) fusion with the RNA binding domain of a first dCsy4 (dCsy4-1) and the RNA binding domain of a second dCsy4 (dCsy4-2) with an sesPN modified to include the dCsy4-1 RNA binding site and a casPN modified to include the dCsy4-2 RNA binding site.

The N- or C-terminal end of the Acidaminococcus spp. Cpf1 is fused to the N- or C-terminal end of a first dCsy4 RNA binding domain, or the first dCsy4 (an enzymatically inactive variant from Pseudomonas aeruginosa (UCBPP-PA14)) is fused to a site internal to the Cpf1 protein to form the fusion protein (dCsy4-1)Cpf1. A second dCsy4 RNA binding domain (an enzymatically inactive variant from Dickeya dadantii Ech703) is fused to a site other than the site to which the first dCsy4-1 RNA binding domain is fused to form the fusion protein (dCsy4-1)Cpf1(dCsy4-2). Examples of insertion sites in Cpf1 to insert the dCsy4-1 RNA binding domain and the dCsy4-2 RNA binding domain to create (dCsy4-1)Cpf1(dCsy4-2) fusion proteins are presented in Table 9. Numerous pairs of Csy4 protein/Csy4 protein binding site are known in the art (e.g., FIG. 5 of U.S. Pat. No. 9,115,348, Haurwitz, R., et al.).

TABLE 9

| Residue number in | |
|---|---|
| Acidaminococcus spp. | |
| Cpf1 | Inserted Domain |
| N1090 | dCsy4-1 |
| T402 | dCsy4-1 |
| D1208 | dCsy4-1 |
| R1194 | dCsy4-1 |
| E441 | dCsy4-1 |
| R840 | dCsy4-2 |
| N-terminus | dCsy4-2 |
| C-terminus | dCsy4-1 |

sesRNA is designed to include a Csy4 hairpin RNA at the 3′ end, wherein the Csy4 hairpin RNA is the RNA binding site for the first dCsy4-1. The Csy4-1 hairpin is connected to the sesRNA using RNA linkers of various lengths (e.g., 10-40 bases) to produce the Csy4-1 tagged sesRNA (Csy4-1)-sesRNA. casRNA is designed to include a Csy4 hairpin RNA at the 5′ end, or at a site internal to the casRNA, wherein the Csy4 hairpin RNA is the RNA binding site to the first dCsy4-2. The Csy4-2 hairpin is connected to the casRNA using RNA linkers of various lengths (e.g., 0-40 bases) to produce the Csy4-2 tagged casRNA ((Csy4-2)-casRNA).

For the biochemical cleavage reaction the (dCsy4-1)Cpf1(dCsy4-2) fusion protein is incubated with a (Csy4-1)-sesRNA. The resulting (dCsy4-1)Cpf1 (dCsy4-2)/(Csy4-1)-sesRNA complexes are incubated with a (Csy4-2)-casRNA-3 to form the (dCsy4-1)Cpf1 (dCsy4-2)/(Csy4-1)-sesRNA/(Csy4-2)-casRNA-3 RNP complex. The biochemical cleavage reaction is performed as previously described (Example 3).

These data are used to demonstrate that the (dCsy4-1)Cpf1(dCsy4-2)/(Csy4-1)-sesPN/(Csy4-2)-casPN nucleoprotein complex constructs as described herein facilitate Cas protein mediate site-specific cleavage of target double-stranded DNA.

Following the guidance of the present specification and examples, the Cas cleavage assay described in this example can be practiced by one of ordinary skill in the art with other CRISPR-Cas proteins, including, but not limited to Cas9 proteins, Cas9-like proteins, proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof, as well as their cognate polynucleotide components.

As is apparent to one of skill in the art, various modification and variations of the above embodiments can be made without departing from the spirit and scope of this invention. Such modifications and variations are within the scope of this invention.

What is claimed is:

1. A Class 2 CRISPR-Cas nucleoprotein complex, comprising:
a Class 2 CRISPR-Cas protein and a Class 2 CRISPR-Cas associated polynucleotide lacking a spacer element (casPN); and
a distinct spacer element sequence polynucleotide (sesPN) comprising a target nucleic acid binding sequence;
wherein the Class 2 CRISPR-Cas nucleoprotein complex is capable of site-directed binding to a target nucleic acid complementary to the target nucleic acid binding sequence of the sesPN.

2. The Class 2 CRISPR-Cas nucleoprotein complex of claim 1, wherein the casPN comprises RNA.

3. The Class 2 CRISPR-Cas nucleoprotein complex of claim 2, wherein the sesPN comprises DNA, RNA, or a combination thereof.

4. The Class 2 CRISPR-Cas nucleoprotein complex of claim 1, wherein the sesPN comprises DNA, RNA, or a combination thereof.

5. The Class 2 CRISPR-Cas nucleoprotein complex of claim 1, wherein the Cas protein comprises a Cas9 protein.

6. The Class 2 CRISPR-Cas nucleoprotein complex of claim 1, wherein the Cas protein comprises a Cpf1 protein.

7. The Class 2 CRISPR-Cas nucleoprotein complex of claim 1, wherein the Cas protein comprises a dCas protein.

8. The Class 2 CRISPR-Cas nucleoprotein complex of claim 1, wherein (i) the sesPN further comprises a nucleic acid binding protein binding sequence, and (ii) the Cas protein comprises a fusion protein comprising the Cas protein and a nucleic acid binding protein domain that binds the nucleic acid binding protein binding sequence of the sesPN.

9. The Class 2 CRISPR-Cas nucleoprotein complex of claim 8, wherein the nucleic acid binding protein domain comprises a dCsy4 protein and the nucleic acid binding protein binding sequence of the sesPN comprises a Csy4 RNA binding sequence.

10. The Class 2 CRISPR-Cas nucleoprotein complex of claim 1, wherein (i) the casPN further comprises a nucleic acid binding protein binding sequence, and (ii) the Cas protein comprises a fusion protein comprising the Cas protein and a nucleic acid binding protein domain that binds the nucleic acid binding protein binding sequence of the casPN.

11. The Class 2 CRISPR-Cas nucleoprotein complex of claim 10, wherein the nucleic acid binding protein domain comprises a dCsy4 protein and the nucleic acid binding protein binding sequence of the casPN comprises a Csy4 RNA binding sequence.

12. The Class 2 CRISPR-Cas nucleoprotein complex of claim 1, wherein (i) the Cas protein comprises an engineered Cas protein comprising a Cys substitution of a non-Cys amino acid residue, (ii) the sesPN comprises a thiol cross-linking moiety, and (iii) the engineered Cas protein substituted Cys amino acid residue is covalently bound to the sesPN thiol cross-linking moiety.

13. The Class 2 CRISPR-Cas nucleoprotein complex of claim 12, wherein the thiol cross-linking moiety is selected from the group consisting of 5′ thiol C6, dithiol phosphoramidite, and 3′ thiol C3.

14. The Class 2 CRISPR-Cas nucleoprotein complex of claim 12, wherein (i) the casPN further comprises a nucleic acid binding protein binding sequence, and (ii) the Cas protein comprises a fusion protein comprising the Cas protein and a nucleic acid binding protein domain that binds the nucleic acid binding protein binding sequence of the casPN.

15. The Class 2 CRISPR-Cas nucleoprotein complex of claim 1, wherein (i) the Cas protein comprises an engineered Cas protein comprising a Cys substitution of a non-Cys amino acid residue, (ii) the casPN comprises a thiol cross-linking moiety, and (iii) the engineered Cas protein substituted Cys amino acid residue is covalently bound to the casPN thiol cross-linking moiety.

16. The Class 2 CRISPR-Cas nucleoprotein complex of claim 15, wherein the thiol cross-linking moiety is selected from the group consisting of 5′ thiol C6, dithiol phosphoramidite, and 3′ thiol C3.

17. The Class 2 CRISPR-Cas nucleoprotein complex of claim 15, wherein (i) the sesPN further comprises a nucleic acid binding protein binding sequence, and
(ii) the Cas protein comprises a fusion protein comprising the Cas protein and a nucleic acid binding protein domain that binds the nucleic acid binding protein binding sequence of the sesPN.

18. A method of cutting a target nucleic acid, comprising:
contacting a nucleic acid comprising the target nucleic acid with the Class 2 CRISPR-Cas nucleoprotein complex of claim 1, thereby facilitating binding of the Class 2 CRISPR-Cas nucleoprotein complex to the target nucleic acid, wherein the bound Class 2 CRISPR-Cas nucleoprotein complex cuts the target nucleic acid.

19. The method of claim 18, wherein the Cas protein of the Class 2 CRISPR-Cas nucleoprotein complex is selected from the group consisting of a Cas9 protein and a Cpf1 protein.

20. A kit comprising:
the Class 2 CRISPR-Cas nucleoprotein complex of claim 1, and
a buffer.
